SEQUENCE LISTING (16 sequences)

SEQ ID NO: 1 - DNA, 20 bp, Artificial Sequence
  Primer
tcctttgtcagtcatgctca

SEQ ID NO: 2 - DNA, 20 bp, Artificial Sequence
  Primer
gttacataggaagcccggat

SEQ ID NO: 3 - DNA, 20 bp, Artificial Sequence
  Primer
tgggaatgtgtctttatcca

SEQ ID NO: 4 - DNA, 22 bp, Artificial Sequence
  Primer
agctctaacatagaggccagat

SEQ ID NO: 5 - DNA, 23 bp, Artificial sequence
  Primer
tctgaccaaaataccaaaagaac

SEQ ID NO: 6 - DNA, 22 bp, Artificial Sequence
  Primer
agagactccagatgtttgatga

SEQ ID NO: 7 - DNA, 23 bp, Artificial Sequence
  Primer
gtctcagtctccaggtgacaatg

SEQ ID NO: 8 - DNA, 22 bp, Artificial Sequence
  Primer
ttcatcttactcaaagtggggc

SEQ ID NO: 9 - DNA, 20 bp, Artificial Sequence
  Primer
ctgaggtagggggatggctt

SEQ ID NO: 10 - DNA, 25 bp, Artificial sequence
  Primer
agctgtgaaaaatcacatattgcta

SEQ ID NO: 11 - DNA, 18 bp, Artificial Sequence
  Primer
atgcaatgagtgctccct

SEQ ID NO: 12 - DNA, 20 bp, Artificial Sequence
  Primer
tgatgatgctcaaatgatgg

SEQ ID NO: 13 - DNA, 20 bp, Artificial Sequence
  Primer
gagtagtgtcatggctccct

SEQ ID NO: 14 - DNA, 20 bp, Artificial Sequence
  Primer
agatgttcagttccttggct

SEQ ID NO: 15 - DNA, 23 bp, Artificial Sequence
  Primer
tccaaggagctgcaggcggcgca

SEQ ID NO: 16 - DNA, 31 bp, Artificial Sequence
  Primer
acagaattcgccccggcctggtacactgcca

(19) United States
(12) Patent Application Publication
(10) Pub. No.: US 2003/0170678 A1
(43) Pub. Date: Sep. 11, 2003

(54) Genetic markers for Alzheimer's disease and methods using the same

(75) Inventors: Rudolph E. Tanzi, Hull, MA (US); Kenneth David Becker, San Diego, CA (US); Gonul Velicelebi, San Diego, CA (US); Kathryn J. Elliott, San Diego, CA (US); Xin Wang, San Diego, CA (US); Lars Bertram, Brighton, MA (US); Aleister J. Saunders, Philadelphia, PA (US); Deborah Lynne Blacker, Newton, MA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignee: Neurogenetics, Inc.

(21) Appl. No.: 10/281,456

(22) Filed: Oct. 25, 2002

Related U.S. Application Data
(60) Provisional application No. 60/348,065, filed on Oct. 25, 2001.
(60) Provisional application No. 60/336,983, filed on Nov. 2, 2001.

(51) Int. Cl. C12Q 1/68

(52) U.S. Cl. 435/6

(57) ABSTRACT

Genetic markers associated with Alzheimer's disease are provided. Also provided are methods of determining the presence or absence in a subject of one or more polymorphisms associated with Alzheimer's disease and methods of determining the level of risk for Alzheimer's disease in a subject. Further provided are nucleic acid compositions and kits for use in determining the presence or absence in a subject of one or more polymorphisms associated with Alzheimer's disease and kits for determining the level of risk for Alzheimer's disease in a subject.

### RELATED APPLICATIONS

Benefit of priority under §119(e) is claimed to U.S. provisional application Serial No. 60/348,065, filed Oct. 25, 2001, entitled “Genetic Markers for Alzheimer's Disease and Methods of Using the Same,” and to U.S. provisional application Serial No. 60/336,983, filed Nov. 2, 2001, entitled “Genetic Markers for Alzheimer's Disease and Methods of Using the Same”. This application is also related to U.S. provisional application Serial No. 60/339,525, filed Oct. 25, 2001, entitled “Genes and Polymorphisms on Chromosome 10 Associated with Alzheimer's Disease and Other Neurodegenerative Diseases,” to U.S. provisional application Serial No. 60/338,010, filed Nov. 8, 2001, entitled “Genes and Polymorphisms on Chromosome 10 Associated with Alzheimer's Disease and Other Neurodegenerative Diseases,” to U.S. provisional application Serial No. 60/368,919, filed Mar. 28, 2002, entitled “Genes and Polymorphisms on Chromosome 10 Associated with Alzheimer's Disease and Other Neurodegenerative Diseases,” to U.S. provisional application Serial No. 60/336,929, filed Nov. 8, 2001 entitled “POLYMORPHIC UROKINASE PLASMINOGEN ACTIVATOR GENES”, to U.S. provisional application Serial No. 60/338,363, filed Nov. 9, 2001, entitled “POLYMORPHIC UROKINASE PLASMINOGEN ACTIVATOR GENES”, to U.S. provisional application Serial No. 60/337,052, filed Dec. 4, 2001, entitled “POLYMORPHIC UROKINASE PLASMINOGEN ACTIVATOR GENES”, and to Intl. PCT application No. (docket no. 37481-3308PC), filed the same day herewith, entitled “Genes and Polymorphisms on Chromosome 10 Associated with Alzheimer's Disease and Other Neurodegenerative Diseases.” The subject matter of each of these applications is incorporated herein in its entirety.

Subject matter of this application was made in part with support from the United States Government under Grant Nos. 1 R01 MH60009 (NIMH) and 5P5OAG05134 (NIA). Thus, the U.S. Government may retain certain rights in such subject matter.

### FIELD OF THE INVENTION

The present invention relates to genetic markers associated with Alzheimer's disease. The invention also relates to methods of determining the presence or absence in a subject of one or more polymorphisms associated with Alzheimer's disease and methods of determining the level of risk for Alzheimer's disease in a subject. The invention further relates to nucleic acid compositions and kits for use in determining the presence or absence in a subject of one or more polymorphisms associated with Alzheimer's disease and kits for determining the level of risk for Alzheimer's disease in a subject.

### BACKGROUND OF THE INVENTION

Alzheimer's disease (AD) is a devastating progressive neurodegenerative disorder, which is the predominant cause of dementia in people over 65 years of age. Clinical symptoms of the disease typically begin with subtle short term memory problems. As the disease progresses, difficulty with memory, language and orientation worsen to the point of interfering with the ability of the person to function independently. Other symptoms, which are variable, include myoclonus and seizures. Duration of AD from the first symptoms of memory loss until death is 10 years on average.

AD is accompanied by a constellation of neuropathological features. The AD brain is characterized by the presence of amyloid-containing plaques and neurofibrillary tangles (NFT). A major molecular component of amyloid is a highly hydrophobic peptide called Aβ peptide. Aβ is an ˜4.0 kDa protein, about 39-43 amino acids long, that is derived from a C-terminal region of amyloid precursor protein (APP). APP is a membrane-spanning glycoprotein that, in one processing pathway, is cleaved inside the Aβ domain to produce α-sAPP, a nonamyloidogenic secreted form of APP. Aβ aggregates into antiparallel filaments in a β-pleated sheet structure resulting in the birefringent nature of the AD amyloid. Although Aβ is the major component of AD amyloid, other proteins have also been found associated with amyloid plaques, e.g., alpha-1-anti-chymotrypsin [Abraham et al. (1988) Cell 52:487-501], cathepsin D [Cataldo (1990) et al. Brain Res. 513:181-192], non-amyloid component protein [Ueda et al. (1993) Proc. Natl. Acad. Sci. U.S.A. 90:11282-11286], apolipoprotein E (apoE) [Namba et al. (1991) Brain Res. 541:163-166; Wisniewski and Frangione (1992) Neurosci. Lett. 135:235-238; Strittmatter et al. (1993) Proc. Natl. Acad. Sci. U.S.A. 90:1977-1981], apolipoprotein J [Choi-Mura (1992) et al. Acta Neuropathol. 83:260-264; McGeer (1992) et al. Brain Res. 579:337-341], heat shock protein 70 [Hamos et al. (1991) Neurology 41:345-350], complement components [McGeer and Rogers (1992) Neurology 43:447-449], alpha2-macroglobin [Strauss et al. (1992) Lab. Invest. 66:223-230], interleukin-6 [Strauss et al. (1992) Lab. Invest. 66:223-230], proteoglycans [Snow et al. (1987) Lab. Invest. 58:454-458], and serum amyloid P [Coria et al. (1988) Lab. Invest. 58:454-458].

Plaques are often surrounded by astrocytes and activated microglial cells expressing immune-related proteins, such as the MHC class II glycoproteins HLA-DR, HLA-DP and HLA-DQ, as well as MHC class I glycoproteins, interleukin-2 (IL-2) receptors and IL-1. Also surrounding many plaques are dystrophic neurites, which are nerve endings containing abnormal filamentous structures.

The characteristic Alzheimer's NFTs contain abnormal filaments bundled together in neurons and occupying much of the perinuclear cytoplasm. These filaments contain the microtubule-associated protein tau in a hyperphosphorylated form. “Ghost” NFTs are also observed in AD brains, which presumably mark the location of dead neurons. Other neuropathological features include granulovascular changes, neuronal loss, gliosis and the variable presence of Lewy bodies.

The destructive process of the disease is evident on a gross level in the AD brain to the extent that in late-stage AD, ventricular enlargement and shrinkage of the brain can be observed by magnetic resonance imaging. The cells remaining at autopsy, however, are grossly different from those of a normal brain, characterized by extensive gliosis and neuronal loss. Neurons which were possibly involved in initiating events, are absent; and other cell types, such as the activated microglial cells and astrocytes, have gene expression patterns not observed in the normal brain. Thus, the amyloid plaque structures and NFTs observed at autopsy are most likely the end-products of a lengthy disease process, far removed from the initiating events of AD.

Accordingly, attempts to use biochemical methods to identify key proteins and genes in the initiating steps of the disease are hampered by the fact that it is not possible to actually observe these critical initiating events. Biochemical dissection of the postmortem AD brain is an analysis of products that occur late in the disease long after the early pathogenic processes associated with AD had begun. Reconstruction of the pathogenic pathways of AD through biochemical analysis of lesions of the postmortem AD brain has its limitations and has been referred to as “molecular archaeology” [Schellenberg (1995) Proc. Natl. Acad. Sci. U.S.A. 92:8552-8559].

The diagnosis of AD is confounded with other dementing diseases and conditions common in the elderly, including dementia-causing conditions such as strokes, microvascular disease, brain tumors, thyroid dysfunction, drug reactions, severe depression and a host of other conditions that can cause intellectual deficits in the elderly. Furthermore, many of the pathological features of AD are not unique to the disease and also occur in the brains of normal aged individuals and are associated with diseases such as Guam Parkinson disease, dementia pugilistica and progressive supranuclear palsy. For example, the twisted filaments that form NFTs also occur in certain tangles associated with other diseases such as Pick's Disease.

AD is a genetically complex and heterogeneous disorder. Because AD is relatively common in the elderly, clustering of cases in a family may occur by chance, representing possible confounding non-allelic genetic heterogeneity, or etiologic heterogeneity with genetic and non-genetic cases co-existing in the same kindred. Despite these problems, it has been found that roughly 40% of early-onset AD (less than 65 years of age) is attributable to missense mutations in three genes (APP, PS1 and PS2). In contrast, the genetic basis of late onset AD has proven more difficult to disentangle [Blacker and Tanzi (1998) Arch. Neurol. 55:294]. Substantial evidence suggests that inherited genetic defects are involved in late-onset AD. Families with multiple late-onset AD cases have been described [Bird et al. (1989) Ann. Neurol. 25:12-25; Heston and White (1978) Behavior Genet. 8:315-331; Pericak-Vance et al. (1988) Exp. Neurol. 102:271-279].

To date, only one genetic risk factor, a common polymorphism in the apolipoprotein E (APOE) gene, referred to as the ε4 allele (APOE4), has been replicated in independent samples in late-onset AD [Farrer et al. (1997) J. Am. Med. Assoc. 278:1349-1356; Blacker et al. (1997) Neurology 48:139]. However, approximately half of the people who develop AD do not carry the APOE ε4 allele found in several other families with high incidence of AD, including the Volga German (VG) kindreds [Brousseau et al. (1994) Neurology 44:342-344; Kuusisto et al. (1994) Brit. Med. J. 309:363; Tsai et al. (1994) Am. J. Hum. Genet. 54:643; Liddel et al. (1994) J. Med. Genet. 31:197; Cook et al. (1979) Neurology 29:1402-1412; Bird et al. (1988) Ann. Neurol. 23:25-31; Bird et al. (1989) Ann. Neurol. 25:12-25]. The known AD loci have been excluded as possible causes of the discrepancy [Schellenberg et al. (1992) Science 258:668; Lannfelt et al. (1993) Nat. Genet. 4:218-219; van Duijn et al. (1994) Am. J. Hum. Genet. 55:714-727; Schellenberg et al. (1988) Science 241:1507; Schellenberg et al. (1991) Am. J. Hum. Genet. 48:563; Schellenberg et al. (1991) Am. J. Hum. Genet. 49:511-517; Kamino et al. (1992) Am. J. Hum. Genet. 51:998; Schellenberg et al. (1993) Am. J. Hum. Genet. 53:619; Schellenberg et al. (1992) Ann. Neurol. 31:223; Yu et al. (1994) Am. Hum. Genet. 54:631]. Furthermore, not all people who do carry the APOE4 allele develop AD, even if they live to old age.

There is evidence that genetic factors other than APOE4 contribute to the risk for late onset AD. A study modeling AD as a quantitative trait estimated that there are at least four additional genetic susceptibility loci for the disease [Daw et al. (2000) Am. J. Hum. Genet. 66:196]. Thus, there is a need to identify genetic markers linked to AD genes, the locations of AD genes and the particular disease genes and mutations therein. Useful genetic markers and mutations will greatly facilitate detection of a predisposition for AD and the development of therapeutics that target AD gene-related alterations.

### SUMMARY OF THE INVENTION

Provided herein are genetic markers associated with Alzheimer's disease (AD). Genetic markers associated with AD are located on chromosome 10. Particular markers associated with AD are located on chromosome 10q. Included among the genetic markers associated with AD provided herein are markers located on chromosome 10q22, 10q23, 10q24, 10q25 and 10q26. In particular embodiments, the markers are located on human chromosome 10. Genetic markers provided herein are associated with AD such that they provide scores or results indicative of linkage disequilibrium with an AD DNA segment (e.g., an AD gene) or association with an allele that confers protection against AD, or of association with AD when tested in linkage disequilibrium or association assessment methods described herein or known to those of skill in the art for association with an allele that confers risk for or protection against AD. Genetic markers are provided that are in linkage disequilibrium with an allele that confers risk for or protection against AD. The genetic markers are associated with AD as individual markers and/or in combinations, such as haplotypes, that are associated with AD.

In further embodiments of the genetic markers associated with AD provided herein, the markers are located on chromosome 10q22, 10q23 or 10q24 or on chromosome 10q23, 10q24 or 10q25. The genetic markers may, for example, be located in a region of chromosome 10 about 30 cM around and including D10S583, or about 20 cM around and including D10S583, or about 15 cM around and including D10S583, or about 12 cM around and including D10S583, or about 10 cM around and including D10S583. In particular embodiments, the genetic markers may be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. The genetic markers may also be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In further embodiments, the genetic markers may be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

Genetic markers associated with AD provided herein may also be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In particular embodiments of the genetic markers associated with AD provided herein, the markers are located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Also provided herein are combinations or haplotypes of genetic markers which are associated with AD. In one embodiment, each genetic marker in a combination is associated with AD. In other embodiments, some of the genetic markers in the combination are associated with AD and some of the genetic markers are not or none of the genetic markers is associated with AD. In such embodiments, the combination of markers as a whole is associated with AD, such as in the case of a haplotype.

Genetic markers contained within the combinations of genetic markers associated with AD provided herein are located on chromosome 10. In particular embodiments, the genetic markers are located on human chromosome 10. In further embodiments of the combinations of genetic markers associated with AD, the genetic markers are located on chromosome 10q. For example, each of the genetic markers in the combination may be located on chromosome 10q22, 10q23, 10q24, 10q25 or 10q26. In particular embodiments, each of the genetic markers may be located on chromosome 10q22, 10q23 or 10q24 or on chromosome 10q23, 10q24 or 10q25. In further embodiments of the combinations of genetic markers associated with AD, each of the genetic markers may be located in a region of chromosome 10 about 30 cM around and including D10S583, or about 20 cM around and including D10S583, or about 15 cM around and including D10S583, or about 12 cM around and including D10S583, or about 10 cM around and including D10S583. Each of the genetic markers may also be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. In yet further embodiments, each of the genetic markers may be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. Each of the genetic markers of the combinations of genetic markers associated with AD may be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In other embodiments of the combinations of genetic markers associated with AD provided herein, each of the genetic markers may be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In particular embodiments of the combinations of genetic markers provided herein, each of the genetic markers may be located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Genetic markers provided herein may be used in a variety of methods. Provided herein are methods involving polymorphisms, such as genetic markers, associated with AD. In particular embodiments of each of the methods provided herein, the genetic markers or polymorphisms or alleles associated with AD are located on human chromosome 10. Such methods include a method for detecting the presence or absence in a subject of a polymorphism associated with Alzheimer's disease in which chromosome 10 of the subject is analyzed for a polymorphism, or combination or haplotype of polymorphisms, associated with Alzheimer's disease. The polymorphism, or each of the polymorphisms of the combination, may be located on chromosome 10q, and in particular, on chromosome 10q22, 10q23, 10q24, 10q25 or 10q26. In a further embodiment, the polymorphism, or each of the polymorphisms in the combination, may be located on chromosome 10q22, 10q23 or 10q24 or on chromosome 10q23, 10q24 or 10q25. The polymorphism, or each of the polymorphsims in the combination, may be located in a region of chromosome 10 about 30 cM around and including D10S583, or about 20 cM around and including D10S583, or about 15 cM around and including D10S583, or about 12 cM around and including D10S583, or about 10 cM around and including D10S583. The polymorphism, or each of the polymorphisms in the combination, may be located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. These methods may also be practiced wherein the polymorphism, or each of the polymorphisms in the combination, is located about 30 Mb around and including D10S583, or about 28 Mb around and including D10S583, or about 20 Mb around and including D10S583, or about 15 Mb around and including D10S583, or about 10 Mb around and including D10S583, or about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In a further embodiment of the method, the polymorphism, or each of the polymorphisms in the combination, is located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In a particular embodiment of the method, the polymorphism, or each of the polymorphisms in the combination, is located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Also provided are embodiments of the method wherein the polymorphism is an allele of D10S583.

Further provided are embodiments of any of the methods for detecting the presence or absence in a subject of a polymorphism, or combination or haplotype of polymorphisms, associated with Alzheimer's disease, wherein the polymorphism, or combination or haplotype of polymorphisms, may be associated with AD with onset ages of greater than or equal to about 50 years, or greater than or equal to about 60 years, or greater than or equal to about 65 years. The method may also be such that the polymorphism, or combination or haplotype of polymorphisms, is associated with an AD DNA segment that has an effect size comparable to or greater than the effect size of APOE. The method may further be such that the polymorphism, or each or some of the polymorphisms of the combination, is located within an AD DNA segment.

Also provided are embodiments of any of the methods for detecting the presence or absence in a subject of a polymorphism, or combination or haplotype of polymorphisms, associated with Alzheimer's disease, in which the association between the polymorphism, or combination or haplotype, and Alzheimer's disease is such that it yields a positive result (i.e., a result indicative of association or linkage disequilibrium) in a family-based test for association. The positive result may be a P value less than or equal to 0.05 or a value less than 0.05.

Further provided are embodiments of any of the methods for detecting the presence or absence in a subject of a polymorphism, or combination or haplotype of polymorphisms, associated with Alzheimer's disease, in which the polymorphism, or combination or haplotype, can be associated with an AD gene that accounts for greater than 1% of the attributtable risk of AD; or the polymorphism may be associated with an AD gene that accounts for greater than 2% of the attributtable risk for AD; or the the polymorphism may be associated with an AD gene that accounts for greater than 5% of the attributtable risk for AD; or the polymorphism may be associated with an AD gene that accounts for greater than 10% of the attributtable risk for AD; or the polymorphism may be associated with an AD gene that accounts for greater than 25% of the attributtable risk for AD.

Also provided are embodiments of any of the methods for detecting the presence or absence in a subject of a polymorphism, or combination or haplotype of polymorphisms, associated with Alzheimer's disease, in which the polymorphism, or at least one of the polymorphisms of the combination, is located in one of the following genes: PLAU, TLL2, PSAP, PSD, KIAA0904, NFKB2, PPP3CB, CH25H and FERIL3.

Also provided is a method for detecting the presence or absence in a subject of polymorphisms associated with Alzheimer's disease that includes a step of analyzing chromosome 10 of the subject for two or more polymorphisms associated with Alzheimer's disease, wherein at least two of the polymorphisms are associated with different AD DNA segments or wherein at least one polymorphism is D10S583. In particular embodiments of these methods, the two or more polymorphisms may be located on chromosome 10q. For example, each of the two or more polymorphisms may be located on chromosome 10q22, 10q23, 10q24, 10q25 or 10q26. Each of the two or more polymorphisms may be located on chromosome 10q22, 10q23 or 10q24 or on chromosome 10q23, 10q24 or 10q25.

In particular embodiments of the method for detecting the presence or absence in a subject of polymorphisms associated with Alzheimer's disease, each of the two or more polymorphisms may be located in a region of chromosome 10 about 30 cM around and including D10S583, or about 20 cM around and including D10S583, or about 15 cM around and including D10S583, or about 12 cM around and including D10S583, or about 10 cM around and including D10S583. In further embodiments of the method, each of the two'or more polymorphisms may be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. In yet further embodiments of the method, each of the two or more polymorphisms may be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. Each of the two or more polymorphisms may be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In other embodiments of the method for detecting the presence or absence in a subject of polymorphisms associated with Alzheimer's disease, each of the two or more polymorphisms may be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In yet further embodiments of the method for detecting the presence or absence in a subject of polymorphisms associated with Alzheimer's disease, each of the two or more polymorphisms may be located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Further provided are embodiments of any of the methods for detecting the presence or absence in a subject of polymorphisms associated with Alzheimer's disease, wherein at least one of the polymorphisms may be associated with AD with onset ages of greater than or equal to about 50 years, or greater than or equal to about 60 years, or greater than or equal to about 65 years. The method may also be such that at least one of the polymorphisms is associated with an AD DNA segment that has an effect size comparable to or greater than the effect size of APOE. The method may further be such that at least one of the polymorphisms is located within an AD DNA segment.

Also provided are embodiments of any of the methods for detecting the presence or absence in a subject of polymorphisms associated with Alzheimer's disease, in which the association between each polymorphism and Alzheimer's disease is such that it yields a positive result (i.e., a result indicative of association or linkage disequilibrium) in a family-based test for association. The positive result may be a P value less than or equal to 0.05 or a value less than 0.05.

Further provided are embodiments of any of the methods for detecting the presence or absence in a subject of polymorphisms associated with Alzheimer's disease, in which at least one of the polymorphisms can be associated with an AD gene that accounts for greater than 1% of the attributtable risk of AD; or the polymorphism may be associated with an AD gene that accounts for greater than 2% of the attributtable risk for AD; or the the polymorphism may be associated with an AD gene that accounts for greater than 5% of the attributtable risk for AD; or the polymorphism may be associated with an AD gene that accounts for greater than 10% of the attributtable risk for AD; or the polymorphism may be associated with an AD gene that accounts for greater than 25% of the attributtable risk for AD.

Also provided are embodiments of any of the methods for detecting the presence or absence in a subject of polymorphisms associated with Alzheimer's disease, in which at least one of the polymorphisms is located in one of the following genes: PLAU, TLL2, PSAP, PSD, KIAA0904, NFKB2, PPP3CB, CH25H and FERIL3. In such methods, one of the polymorphisms may be located in one of the identified genes and another of the polymorphisms may be located in another of the identified genes.

A method of determining the level of risk for Alzheimer's disease in a subject is also provided. Such methods include a step of analyzing chromosome 10 of the subject for the presence or absence of one or more polymorphisms associated with Alzheimer's disease. Methods of determining the level of risk for Alzheimer's disease in a subject may include a step of analyzing chromosome 10 of a subject for the presence or absence of a combination or haplotype of polymorphisms associated with AD. In any of these methods, the one or more polymorphisms or the combination of polymorphisms can be such that they are indicative of an increased or decreased risk for Alzheimer's disease. In particular methods of determining the level of risk for AD in a subject, the association between the polymorphism or combination of polymorphisms and Alzheimer's disease yields a result in a family-based test for association that is indicative of linkage disequilibrium between the polymorphism(s) and an allele associated with Alzheimer's disease. The association between the polymorphism or combination of polymorphisms and Alzheimer's disease may yield a positive result in a family-based test for association. The positive result may be a P value less than or equal to 0.05. In particular embodiments, the positive result is a P value less than 0.05.

In further embodiments of these methods, the polymorphism or combination of polymorphisms may be associated with unaffected members of a family having members affected with Alzheimer's disease. The polymorphism or combination or polymorphisms may be indicative of a decreased risk for Alzheimer's disease. The association between the polymorphism or combination and unaffected members of a family having members affected with Alzheimer's disease yields a positive result in a family-based test for association. The positive result may be a P value less than or equal to 0.05. In particular embodiments, the positive result is a P value less than 0.05.

In yet further embodiments of the method, the polymorphism or combination of polymorphisms may be under-represented in cases of a case-control study. In particular embodiments of the method of determining the level of risk for Alzheimer's disease in a subject, the polymorphism is an allele of D10S583 and the presence of the allele of D10S583 is indicative of a decreased risk for AD. The allele of D10S583 may be about 210 bp. The allele of D10S583 may be 209 bp or 211 bp.

In other embodiments of the method of determining the level of risk for Alzheimer's disease in a subject, the polymorphism or combination of polymorphisms may be associated with affected members of a family having members affected with Alzheimer's disease and is indicative of an increased risk for Alzheimer's disease. The association between the polymorphism or combination of polymorphisms and affected members of a family having members affected with Alzheimer's disease may yield a positive result in a family-based test for association. The positive result may be a P value less than or equal to 0.05. In particular, the positive result is a P value less than 0.05.

In yet further embodiments of the method, the polymorphism or combination of polymorphisms is over-represented in cases of a case-control study.

In particular embodiments of the methods of determining the level of risk for AD in a subject, the one or more polymorphisms or combination of polymorphisms associated with AD is located on chromosome 10q. In further embodiments, each of the one or more polymorphisms, or each of the polymorphisms of the combination of polymorphisms, is located on chromosome 10q22, 10q23, 10q24, 10q25 or 10q26. In yet further embodiments of the methods, each of the one or more polymorphisms, or each of the polymorphisms of the combination of polymorphisms, is located on chromosome 10q22, 10q23 or 10q24 or on chromosome 10q23, 10q24 or 10q25.

In particular embodiments of the method of determining the level of risk for AD in a subject, the one or more polymorphisms, or each of the polymorphisms in the combination of polymorphisms associated with AD, may be located in a region of chromosome 10 about 30 cM around and including D10S583, or about 20 cM around and including D10S583, or about 15 cM around and including D10S583, or about 12 cM around and including D10S583, or about 10 cM around and including D10S583. In further embodiments of the methods, each of the one or more polymorphisms, or each of the polymorphisms in the combination of polymorphisms associated with AD, may be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

In yet further embodiments of the methods, each of the one or more polymorphisms, or each of the polymorphisms in the combination of polymorphisms associated with AD, may be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In particular embodiments of the methods, each of the one or more polymorphisms, or each of the polymorphisms in the combination of polymorphisms associated with AD, may be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In particular embodiments of the method of determining the level of risk for AD in a subject, the one or more polymorphisms, or each of the polymorphisms in the combination of polymorphisms associated with AD, may be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In further particular embodiments of the method of determining the level of risk for AD in a subject, the one or more polymorphisms, or each of the polymorphisms in the combination of polymorphisms associated with AD, may be located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Further provided are embodiments of any of the methods for determining the level of risk for AD in a subject wherein at least one of the polymorphisms, or the combination of polymorphisms, is associated with AD with onset ages of greater than or equal to about 50 years, or greater than or equal to about 60 years, or greater than or equal to about 65 years. The method may also be such that at least one of the polymorphisms, or the combination of polymorphisms, is associated with an AD DNA segment that has an effect size comparable to or greater than the effect size of APOE. The method may further be such that at least one of the polymorphisms, or the combination of polymorphisms, is located within an AD DNA segment.

Also provided are embodiments of any of the methods for determining the level of risk for AD in a subject in which at least one of the polymorphisms, or the combination of polymorphisms, can be associated with an AD gene that accounts for greater than 1%, greater than 2%, greater than 5%, greater than 10% or greater than 25% of the attributtable risk for AD.

Also provided are embodiments of any of the methods for determining the level of risk for AD in a subject in which at least one of the polymorphisms, or at least one of polymorphisms in the combination of polymorphisms, is located in one of the following genes: PLAU, TLL2, PSAP, PSD, KIAA0904, NFKB2, PPP3CB, CH25H and FERIL3. In such methods, one of the polymorphisms may be located in one of the identified genes and another of the polymorphisms may be located in another of the identified genes.

Further provided is a method for indicating a predisposition to or the occurrence of Alzheimer's disease in a subject. The method includes a step of detecting in nucleic acid obtained from the subject the presence or absence of a polymorphism on chromosome 10 associated with Alzheimer's disease. Methods for indicating a predisposition to or the occurrence of Alzheimer's disease in a subject may include a step of analyzing chromosome 10 of a subject for the presence or absence of a combination or haplotype of polymorphisms associated with AD. In any of these methods, the presence of the polymorphism or the combination of polymorphisms can be indicative of a predisposition to Alzheimer's disease. In particular embodiments of these methods, the polymorphism or combination of polymorphisms is on human chromosome 10.

Also provided is a method for confirming a phenotypic diagnosis of Alzheimer's disease in a subject. The method includes a step of detecting in nucleic acid obtained from the subject the presence or absence of a polymorphism on chromosome 10 associated with Alzheimer's disease, wherein the presence of the polymorphism confirms a phenotypic diagnosis of Alzheimer's disease. Methods for confirming a phenotypic diagnosis of Alzheimer's disease in a subject may include a step of detecting in nucleic acid obtained from the subject the presence or absence of a combination or haplotype of polymorphisms on chromosome 10 associated with AD. In particular embodiments of these methods, the polymorphism or combination of polymorphisms is on human chromosome 10.

A method for predicting a response of a subject to a drug used to treat Alzheimer's disease is also provided. The method is performed by detecting the presence or absence of at least one polymorphism on chromosome 10 associated with Alzheimer's disease, or a combination or haplotype of polymorphisms on chromosome 10 associated with AD, wherein the presence of the polymorphism or combination is indicative of an increased or decreased likelihood that the drug treatment for Alzheimer's disease will be effective. In particular embodiments of these methods, the polymorphism or combination of polymorphisms is on human chromosome 10.

Also provided is a method of treating a subject manifesting an Alzheimer's disease phenotype. The method is performed by detecting in nucleic acid obtained from the subject the presence of a polymorphism on chromosome 10 associated with Alzheimer's disease, or a combination or haplotype of polymorphisms on chromosome 10 associated with AD, wherein the presence of the polymorphism or combination of polymorphisms is indicative of the occurrence of Alzheimer's disease in a subject; and selecting and administering a treatment that is effective for treating Alzheimer's disease.

In any of the methods of (1) indicating a predisposition to or the occurrence of Alzheimer's disease in a subject, (2) confirming a phenotypic diagnosis of Alzheimer's disease in a subject, (3) predicting a response of a subject to a drug used to treat Alzheimer's disease and (4) treating a subject manifesting an Alzheimer's disease phenotype, the polymorphism or combination or haplotype of polymorphisms may be located on chromosome 10q. In particular embodiments, the polymorphism or combination or haplotype of polymorphisms may be located on chromosome 10q22, 10q23, 10q24, 10q25 or 10q26. In further embodiments the polymorphism, or each polymorphism in the combination or haplotype of polymorphisms, may be located on chromosome 10q22, 10q23 or 10q24 or on chromosome 10q23, 10q24 or 10q25.

In any of these methods, the polymorphism, or each polymorphism in the combination or haplotype of polymorphisms, may be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In any of these methods, the polymorphism, or each polymorphism in the combination or haplotype of polymorphisms, may be located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

These methods can also be practiced where the polymorphism, or each polymorphism in the combination of polymorphisms, may be located in a region of chromosome 10 about 30 cM around and including D10S583, or about 20 cM around and including D10S583, or about 15 cM around and including D10S583, or about 12 cM around and including D10S583, or about 10 cM around and including D10S583.

In further embodiments of any of these methods, the polymorphism, or each polymorphism in the combination of polymorphisms, may be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

In yet further embodiments of any of these methods, the polymorphism, or each polymorphism in the combination of polymorphisms, may be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

The polymorphism, or each polymorphism in the combination of polymorphisms, may also be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

Also provided are embodiments of any of the methods in which the association between the polymorphism, or a combination of polymorphisms, and Alzheimer's disease is such that it yields a positive result (i.e., a result indicative of association or linkage disequilibrium) in a family-based test for association. The positive result may be a P value less than or equal to 0.05 or a value less than 0.05.

Also provided are embodiments of any of the methods in which at the polymorphism, or the combination of polymorphisms, can be associated with an AD gene that accounts for greater than 1%, greater than 2%, greater than 5%, greater than 10% or greater than 25% of the attributtable risk for AD.

Also provided are embodiments of any of the methods in which at least one polymorphism, or at least one of the polymorphisms in a combination of polymorphisms, is located in one of the following genes: PLAU, TLL2, PSAP, PSD, KIAA0904, NFKB2, PPP3CB, CH25H and FERIL3. In such methods, one of the polymorphisms may be located in one of the identified genes and another of the polymorphisms may be located in another of the identified genes.

A method for identifying a gene as a candidate Alzheimer's disease gene is also provided. The method is performed by selecting a gene on chromosome 10 that is or encodes a product that has one or more properties which relate to one or more phenomena in neurodegenerative disease and thereby identifying a candidate Alzheimer's disease gene. In particular embodiments, the gene is on human chromosome 10.

The gene can be located, for example, on chromosome 10q. In particular embodiments, the gene can be located on chromosome 10q22, 10q23, 10q24, 10q25 or 10q26. In further embodiments of the method, the gene may be located on chromosome 10q22, 10q23 or 10q24, or on chromosome 10q23, 10q24 or 10q25, or located in a region of chromosome 10 about 30 cM around and including D10S583, or about 20 cM around and including D10S583, or about 15 cM around and including D10S583, or about 12 cM around and including D10S583, or about 10 cM around and including D10S583, located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

In other embodiments of these methods, the gene is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In further embodiments of the method, the gene is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In particular embodiments of the method, the gene is located in one or more of the following regions of chromosome 10: (a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In yet further embodiments of the methods, the gene is located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In these methods the one or more phenomena in neurodegenerative disease can be selected from the group consisting of senile plaques and components thereof, neuritic plaques, and components thereof, neurofibrillary tangles, tau protein, abnormally phosphorylated tau protein, amyloid precursor protein (APP), processing of APP, Aβ42 protein, α-, β- and γ-secretases, presenilin proteins, amyloid deposition, Lewy bodies, prions, apoptosis, caspases, inflammation, excitotoxicity, excitotoxins, excessive nitric oxide production, oxidative stress, proteases, protease inhibitors, neurotrophic factors, cytokines, calcium-dependent processes, signal transduction, altered ionic homeostasis, altered calcium homeostasis, synaptic molecules, adhesion molecules, molecules involved in membrane turnover, cholesterol and lipid metabolism and transport, cytoskeletal molecules, neuronal proteins, brain proteins, and cell necrosis.

Also provided is a method for identifying a polymorphism, or combination or haplotype of polymorphisms, associated with Alzheimer's disease. This method may be performed by analyzing a polymorphism on chromosome 10, or a combination of polymorphisms on chromosome 10, for association with Alzheimer's disease. In particular embodiments of the method, the polymorphism is on human chromosome 10.

In further embodiments, the polymorphism, or each of the polymorphisms of the combination of polymorphisms, may be located on chromosome 10q22, 10q23, 10q24, 10q25 or 10q26. In yet further embodiments of the methods, the polymorphism, or each of the polymorphisms of the combination of polymorphisms, may be located on chromosome 10q22, 10q23 or 10q24 or on chromosome 10q23, 10q24 or 10q25.

In particular embodiments of the method for identifying a polymorphism, or combination or haplotype of polymorphisms, associated with Alzheimer's disease, the polymorphism, or each of the polymorphisms in the combination of polymorphisms, may be located in a region of chromosome 10 about 30 cM around and including D10S583, or about 20 cM around and including D10S583, or about 15 cM around and including D10S583, or about 12cM around and including D10S583, or about 10 cM around and including D10S583. In further embodiments of the methods, the polymorphism, or each of the polymorphisms in the combination of polymorphisms, may be located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

In yet further embodiments of the methods, the polymorphism, or each of the polymorphisms in the combination of polymorphisms, may be located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In particular embodiments of the methods, the polymorphism, or each of the polymorphisms in the combination of polymorphisms, may be located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In particular embodiments of the method for identifying a polymorphism, or combination or haplotype of polymorphisms, associated with Alzheimer's disease, the polymorphism, or each of the polymorphisms in the combination of polymorphisms, may be located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In further particular embodiments of the method, the polymorphism, or each of the polymorphisms in the combination of polymorphisms, may be located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Further provided are embodiments of any of the methods for identifying a polymorphism, or combination or haplotype of polymorphisms, associated with Alzheimer's disease, wherein the polymorphism or combination of polymorphisms on chromosome 10 are analyzed for association with Alzheimer's disease with with onset ages of greater than or equal to about 50 years, or greater than or equal to about 60 years, or greater than or equal to about 65 years.

Also provided are embodiments of any of the methods for identifying a polymorphism, or combination or haplotype of polymorphisms, associated with Alzheimer's disease in which the polymorphism, or at least one of polymorphisms in the combination of polymorphisms, is located in one of the following genes: PLAU, TLL2, PSAP, PSD, KIAA0904, NFKB2, PPP3CB, CH25H and FERIL3. In such methods, one of the polymorphisms may be located in one of the identified genes and another of the polymorphisms may be located in another of the identified genes.

Also provided are the above methods where the association between the polymorphism, or the combination of polymorphsims, and Alzheimer's disease yields a positive result in a family-based test for association.

For example, the positive result is a P value less than or equal to 0.05, or is a P value less than 0.05.

Also provided is a combination containing two or more oligonucleotides that hybridize to, adjacent to, or to DNA flanking a DNA segment on chromosome 10, wherein the DNA segment comprises a polymorphism that is associated with AD and at least two of the oligonucleotides hybridize to, adjacent to, or to DNA flanking different DNA segments. In a particular embodiment of the combination, the DNA segment is on chromosome 10q. In further embodiments of the combination, the DNA segment is on chromosome 10q22, 10q23, 10q24, 10q25 or 10q26. In yet further embodiments of the combination, the DNA segment is on chromosome 10q22, 10q23 or 10q24 or on chromosome 10q23, 10q24 or 10q25.

In particular embodiments of the combinations, the DNA segment can be located in any of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In further embodiments of the combinations, the DNA segment can be located in any of the following regions of chromosome 10::

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In the combination, the polymorphism can be, for example, D10S583.

Also provided is a kit containing two or more oligonucleotides that hybridize to, adjacent to, or to DNA flanking a DNA segment on chromosome 10 and instructions describing procedures for using the oligonucleotide(s) in the detection of one or more polymorphisms on chromosome 10 associated with AD, wherein the DNA segment comprises a polymorphic site at which a polymorphism that is associated with AD occurs, and at least two of the oligonucleotides hybridize to, adjacent to, or to DNA flanking different DNA segments.

Further provided is a kit containing two or more oligonucleotides that hybridize to, adjacent to, or to DNA flanking a DNA segment on chromosome 10 and instructions describing procedures for using the oligonucleotide(s) in methods of detecting the presence or absence of one or more polymorphisms on chormosome 10 associated with AD, wherein the DNA segment comprises a polymorphic site at which a polymorphism that is associated with AD occurs, at least two of the oligonucleotides hybridize to, adjacent to, or to DNA flanking different DNA segments, and the procedures provide results on which to base a determination of a predisposition to or occurrence of AD in a subject.

Also provided is a kit containing two or more oligonucleotides that hybridize to, adjacent to, or to DNA flanking a DNA segment on chromosome 10 and instructions describing procedures for using the oligonucleotide(s) in methods of detecting the presence or absence of one or more polymorphisms on chormosome 10 associated with AD, wherein the DNA segment comprises a polymorphic site at which a polymorphism that is associated with AD occurs, at least two of the oligonucleotides hybridize to, adjacent to, or to DNA flanking different DNA segments, and the procedures provide results on which to base a prediction of a subject's response to a treatment for AD.

Further provided is a kit containing two or more oligonucleotides that hybridize to, adjacent to, or to DNA flanking a DNA segment on chromosome 10 and instructions describing procedures for using the oligonucleotide(s) in methods of detecting the presence or absence of one or more polymorphisms on chromosome 10 associated with AD, wherein the DNA segment comprises a polymorphic site at which a polymorphism that is associated with AD occurs, at least two of the oligonucleotides hybridize to, adjacent to, or to DNA flanking different DNA segments, and the procedures provide results on which to base a treatment of AD in a subject.

Also provided is a kit containing one or more oligonucleotides that hybridize to, adjacent to, or to DNA flanking a DNA segment on chromosome 10 and one or more control samples, wherein the DNA segment comprises a polymorphic site at which a polymorphism that is associated with AD occurs, and the one or more control samples comprise the DNA segment in which the polymorphic site does not contain a polymorphism associated with AD and/or the DNA segment in which the polymorphic site does contain a polymorphism associated with AD.

In particular embodiments of any of the kits, the polymorphism or polymorphisms are located on chromosome 10q. In further embodiments of any of the kits, the polymorphism or polymorphisms are located on chromosome 10q22, 10q23, 10q24, 10q25 and/or 10q26. In particular embodiments of the kits, the polymorphism or polymorphisms are located on chromosome 10q22, 10q23 and/or 10q24 or on chromosome 10q23, 10q24 and/or 10q25.

In further embodiments of the kits, the polymorphism may be located in a region of chromosome 10 about 30 cM around and including D10S583, or about 20 cM around and including D10S583, or about 15 cM around and including D10S583, or about 12 cM around and including D10S583, or about 10 cM around and including D10S583. The polymorphism may be located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

In particular embodiments of the kits, the polymorphism is located about 30 Mb around and including D10S583, or about 28 Mb around and including D10S583, or about 20 Mb around and including D10S583, or about 15 Mb around and including D10S583, or about 10 Mb around and including D10S583, or about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In a further embodiments of the kits, the polymorphism is located in one or more of the following regions of chromosome 10:

(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,

(c) the region extending from and including marker D10S583 to the terminus of the q arm of chromosome 10,

(d) the region extending from and including marker D10S583 to the centromere of chromosome 10,

(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,

(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,

(g) the region between D10S564 and D10S583, inclusive,

(h) the region between D10S583 and D10S1710, inclusive,

(i) the region between D10S583 and D10S566, inclusive,

(j) the region between D10S583 and D10S1671, inclusive and

(k) the region between D10S583 and D10S1741, inclusive.

In a particular embodiment of the kits, the polymorphism is located in one or more of the following regions of chromosome 10:

(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,

(b) the region extending about 5 cM, or about 2.5 cM, or about 1 cM, or about 0.5 cM or about 0.1 cM distal (telomeric) from and including marker D10S583,

(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and

(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

### DESCRIPTION OF THE DRAWINGS

FIG. 1 is a list of short tandem repeat polymorphism genetic markers located on human chromosome 10.

FIG. 2 is a list of genes that have been mapped to human chromosome 10. The list includes gene symbols, aliases, approximate cytogenetic locations and accession numbers for locating information, including gene nucleotide sequences, as provided, for example, by the GDB Human Genome Database available on the internet (http://www.gdb.org).

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information is known and can be readiliy accessed, such as by searching the internet and/or appropriate databases. Reference thereto evidences the availability and public dissemination of such information.

As used herein, an “Alzheimer's disease (AD) phenotype” is used interchangeably with “AD trait” and refers to any visible, detectable or otherwise measurable property of an individual diagnosed with AD. Such properties include, but are not limited to, dementia, aphasia (language problems), apraxia (complex movement problems), agnosia (problems in identifying objects), progressive memory impairment, disordered cognitive function, altered behavior, including paranoia, delusions and loss of social appropriateness, progressive decline in language function, slowing of motor functions such as gait and coordination in later stages of AD, amyloid-containing plaques, which are foci of extracellular amyloid-β (Aβ) protein deposition with dystrophic neurites and associated axonal and dendritic injury and microglia expressing surface antigens associated with activation (e.g., CD45 and HLA-DR), diffuse (“preamyloid”) plaques and neuronal cytoplasmic inclusions such as neurofibrillary tangles containing hyperphosphorylated tau protein or Lewy bodies (containing α-synuclein). Standardized clinical criteria for the diagnosis of AD have been established by NINCDS/ADRDA (National Institute of Neurological and Communicative Disorders and Stroke/Alzheimer's Disease and Related Disorders Association) [McKhann et al. (1984) Neurology 34:939-944]. The clinical manifestations of AD as set forth in these criteria are included within the definition of AD phenotype. For example, dementia may be established by clinical exam and documented by any of several neuropsychological tests, including the Mini Mental State Exam (MMSE) [Folstein and McHugh (1975) J. Psychiatr. Res. 12:196-198; Cockrell and Folstein (1988) Psychopharm. Bull. 24:689-692], the Blessed Test [Blessed et al. (1968) Br. J. Psychiatry 114:797-811] and the Alzheimer's Disease Assessment Scale-Cognitive (ADAS-COG) Test [Rosen et al. (1984) Am. J. Psychiatry 141:1356-1364; Weyer et al. (1997) Int. Psychogeriatr. 9:123-138; and Ihl et al. (2000) Neuropsychobiol. 4:102-107].

As used herein, a “property relating to one or more phenomena in neurodegenerative disease” with reference to a gene, gene product or protein refers to a property of the gene or gene product that relates to phenomena which have been widely described and are known to those of skill in the art, including morphological, structural, biological and biochemical occurrences which can be pathophysiological aspects of neurodegenerative diseases. The property may be any aspect or feature of the gene or gene product, including but not limited to its physical composition (e.g., nucleic acids, amino acids, peptides and proteins), functional attributes (e.g., enzymatic capabilities, such as an enzyme catalyst, inhibitory functions, such as enzyme inhibition, antigenic properties, and binding capabilities, such as a receptor or ligand), cellular location(s), expression pattern (e.g., expression in the central nervous system) and/or interactions with other compositions. For example, a property of a gene or gene product relating to one or more phenomena in neurodegenerative disease may be involvement of the gene and/or product thereof in pathways involved in APP metabolism, Aβ protein generation, aggregation and/or degradation, apoptosis, calcium homeostasis, inflammation, oxidative stress, free radical generation, modification of tau protein (e.g., phosphorylation), axonal transport, neuroprotection and neurotrophism.

As used herein, “late-onset” refers to a type of AD in which AD-associated symptoms manifest at an age of ≦about 50 years. In late-onset AD, such symptoms may manifest at any age of about 50 years or older and typically may manifest at ≦about 60 years or ≦about 65 years.

As used herein, “pedigree” refers to a family for which information concerning the ancestral relationships and transmission of genetic traits over several generations is known.

As used herein, “genetic marker” refers to a segment of DNA with an identifiable location on a chromosome. The DNA segment may contain one or more than one nucleotide. The inheritance of a genetic marker may be followed. Typically, genetic markers useful in genetic analyses are polymorphic such that two or more alternative forms or sequences or alleles exist in a population.

As used herein, “allele” refers to variants of a nucleotide sequence. An allele can be one of the variant forms of a gene at a particular locus. Typically, the first identified allele is referred to as the original allele whereas all other alleles are referred to as alternative alleles. Different alleles can produce variation in inherited characteristics. Diploid organisms may be homozygous or heterozygous for an allelic form. In an individual, one form of an allele (the dominant one) may be expressed more than another form (the recessive one).

As used herein, “genotype” refers to the identity of the alleles present in an individual or sample. The term “genotyping” a sample or individual refers to determining a specific allele or specific nucleotide carried by an individual at a marker.

As used herein, “haplotype” refers to a collection of genetic markers. A haplotype can be a combination of alleles present in an individual or sample. A haplotype can be the alleles of different genes received by an individual from one parent, or an array of polymorphisms on a chromosome.

As used herein, “linkage disequilibrium” with reference to the relationship between alleles refers to the deviation from the random occurrence of the alleles in a haplotype in populations. Alleles observed together on a chromosome more often than expected from their frequencies in the population may be referred to as in linkage disequilibrium. Alleles that are physically close are more likely to be inherited together than are alleles that are farther apart. Therefore, variations of several markers that are close to, or within, a particular gene variant on a chromosome are likely to be inherited together with that gene variant when they are in linkage disequilibrium. Thus, genetic markers, e.g., microsatellite markers and SNP variations, that are in linkage disequilibrium and associated with a disease phenotype can mark the position on the chromosome in which a susceptibility gene is located.

Generally, linkage disequilibrium spans chromosome segments ranging in size from about ≦5 kb to about 500 kb or less, such as distances of ≦80 kb or ≦50 kb [Risch (2000) Nature 405:847-856; Abecasis et al. (2001) Am. J. Hum. Genet. 68:191-197; Reich et al. (2001) Nature 41:199-204]. It is common, however, to find some degree of linkage disequilibrium between alleles that are up to about 1-2 cM apart. Significant linkage disequilibrium between microsatellite loci has been reported to extend to ≧4 cM [Huttley et al. (1999) Genetics 152:1711-1722] and as great as ≦˜21 cM [Wilson and Goldstein (2000) Am. J. Hum. Genet. 67:926-935]. The degree of linkage disequilibrium between two alleles can vary based on location within the genome, population distribution, population frequency and demographic history [Reich et al. (2001) Nature 41:199-204; Stephens et al. (2001) Science 293:489-493; Wilson and Goldstein (2000) Am. J. Hum. Genet. 67:926-935].

When a disease-causing allele is in linkage disequilibrium with another allele, the frequency of the other allele will be increased in a disease population as compared to a trait-negative population. This increased frequency is referred to as “genetic association” or “allelic association” between the other allele and the disease. Thus, association between a disease trait and a marker allele can be indicative of linkage disequilibrium between the disease-causing allele and the marker allele. Similarly, when an allele that confers protection against a disease is in linkage disequilibrium with another allele, the frequency of the other allele may be increased in a trait-negative population relative to a disease population. This increased frequency is referred to as genetic association between the other allele and the protective allele.

Studies of genetic association are commonly used to identify genes involved in complex traits. Genetic association studies assess correlations between genetic variants and trait differences on a population scale. In association-based methods of mapping genes that increase susceptibility to disease, evidence is sought for a statistically significant association between an allele and a trait or trait-causing allele. The occurrence of a disease-causing allele may be presumed by the occurrence of the disease trait. In such studies, it may turn out that a significant association is obtained between an allele and a trait-negative population. Such an association may be indicative of linkage disequilibrium between that allele and a protective allele that decreases susceptibility to disease. Association studies focus on population frequencies and explore the relationships among frequencies for sets of alleles between loci. Association may be determined using a number of analytical methods, including but not limited to case-control studies, family-based association techniques and haplotype analyses. Association determinations utilizing alleles that are not transmitted from parents to affected individuals as controls and/or related disease family members (e.g., sib pairs) as affected individuals are particularly useful in accurate determination of association. Such determinations are in contrast to association studies using unrelated populations of subjects and matched controls (e.g., case-control studies), which have the advantage of being relatively simple in terms of the sample sets and statistical analyses involved but may be more susceptible to false positive (type I) and false negative (type I) errors. Thus, in case-control studies, it is possible in some instances (e.g., population stratification, insufficient sample size and/or poorly matched control groups) to observe association in the absence of linkage disequilibrium.

As used herein, an “Alzheimer's disease (AD) DNA segment” or “Alzheimer's disease (AD) gene” is a gene or other DNA segment that either directly causes AD or confers an increased or decreased susceptibility to AD. A gene or DNA segment which causes AD may, for example, have an allele that contains an alteration, e.g., a mutation, relative to another allele(s) of the gene or DNA segment, wherein the alteration can cause or give rise to a defect involved in the manifestation of an AD phenotype.

A gene or DNA segment that confers increased susceptibility to AD may have an allele that predisposes an individual to AD but is not an invariant cause of AD. Thus, an allele that confers increased susceptibility to AD increases the likelihood of developing AD but is not sufficient alone to cause AD. Such an allele may be referred to as a genetic risk ractor for AD and may be one of several genetic risk factors, which in turn may be one type of several types of risk factors. For example, other possible risk factors could include environmental risk factors. An allele of a gene or DNA segment that confers increased susceptibility to AD can be over-represented in cases in case control studies and/or can be associated with affected individuals in a family-based association analysis.

A gene or DNA segment that confers decreased susceptibility to AD can be under-represented in cases in case control studies and/or can be associated with unaffected individuals in a family-based association analysis.

As used herein, a “DNA segment associated with Alzheimer's disease (AD)” refers to an allele that either is an AD gene or DNA segment or is in linkage disequilibrium with an AD gene or AD DNA segment. An allele that is an AD risk factor or AD susceptibility locus may be in linkage disequilibrium with an allele of an AD gene or DNA segment and thus may be a DNA segment associated with AD. DNA segments associated with AD include genes as well as intergenic regions of DNA.

As used herein, the term “protective” with reference to an allele refers to an allele that is indicative of a decreased risk relative to the general population for a genetic disease, e.g., AD. The decreased risk associated with a protective allele may be identified as under-representation of the allele in cases relative to controls, and/or as a significant association between the allele and unaffected members of a family that contains affected members. A protective allele may be a variant of a DNA segment, such as a gene, that has a risk factor or disease allele. A protective allele may be a variant that is “functional” in that it participates in counteracting a defect that occurs in a genetic disease, e.g., AD, or may confer apparent “protection” against a disease by not conferring risk for the disease.

As used herein, the term “penetrance” refers to the ratio between the number of trait positive carriers of a particular allele and the total number of carriers of the allele in the population. Thus, a highly penetrant gene or allele will have a greater penetrance ratio than a weakly or moderately penetrant gene. Penetrance may also be considered as the percent probability that a carrier of a particular allele will express the corresponding phenotype.

As used herein, “prevalence” refers to the percentage of trait positive individuals that carry a particular allele.

The terms “recombination fraction” and “recombination frequency” are used herein interchangeably and refer to the probability of a recombination event between two loci in a genome.

As used herein, “linked” refers to a relationship between two loci in a genome. For example, it may refer to the relationship between a polymorphic or marker site on a chromosome and a gene, such as, for example, a gene associated with AD. The relationship may be defined in a number of ways. For example, the relationship may be defined in terms of the extent to which recombination between the loci occurs. Typically, the transmission of alleles located on different chromosomes occurs in a random fashion through independent assortment. Loci representative of two such alleles are considered to be unlinked. If two loci are situated on the same chromosome, the transmission of alleles of one locus may be affected by the presence of the other locus such that the ratios of alleles are no longer independent, and the loci are referred to as “linked.” Two loci are completely linked when there is no recombination between them; the same alleles or phenotypes are always transmitted together from generation to generation within a family. An intermediate state of linkage, referred to as “incomplete linkage” occurs when the transmission of alleles of two loci deviates consistently and measurably from independent assortment but a consistent recombination fraction nonetheless exists for the loci.

Linkage is commonly assessed by the LOD (logarithm of an odds ratio) score method or other acceptable statistical linkage determination. Positive LOD scores can be considered as evidence of linkage between two loci. The greater the LOD score, the greater the possibility that the loci are linked. LOD scores >1 are particularly indicative of linkage. Classification of linkage has been proposed [see, e.g., Lander and Kruglyak (1995) Nature Genet. 11:241-247] based on the number of times it would be expected to see a result at random in a dense, complete genome scan for linkage. Under such a classification scheme, suggestive linkage is statistical evidence that would be expected to occur one time at random in a genome scan, significant linkage is statistical evidence expected to occur 0.05 times in a genome scan (that is with probability 5%), highly significant linkage is statistical evidence expected to occur 0.001 times in a genome scan and confirmed linkage is significant linkage from one or a combination of initial studies that has subsequently been confirmed in a further sample. In the case of sibling pair-based linkage analysis, for example, suggestive, significant and highly significant linkage may correspond to LOD scores of 2.2, 3.6, and 5.4, respectively.

The relationship between two linked loci may also be defined in terms of the physical or genetic distance between the loci. Thus, two loci may be referred to as linked when they are located relatively close together on the same chromosome. For example, in the case of a polymorphic or marker site on a chromosome linked with a DNA segment associated with AD, the marker may be located a particular number of base pairs (bp) or centiMorgans (cM) from the DNA segment. The particular distance, in bp or cM, between two linked loci can vary, but is small enough so that the linkage score, e.g., the LOD score, obtained in linkage analysis of the two loci (e.g., a marker and a trait such as a disease) is at least indicative of linkage (i.e., the loci are “relatively close” to each other) if not at least suggestive, significant or even highly significant linkage. A linked marker may be within the DNA segment associated with a trait (e.g., AD) and, further, may be a causative polymorphism in a disease (e.g., AD) gene, such as, for example, a polymorphism in an AD gene that is responsible for a defect in an AD gene. When the marker is located within an AD gene, it is referred to as coincident with the gene.

As used herein, the term “effect size” with reference to a disease gene refers to the degree to which mutations or polymorphisms in a DNA segment, e.g., a gene, confer susceptibility to the disease taking into account the magnitude of prevalence and penetrance of the polymorphism.

As used herein, “indicative of a predisposition to Alzheimer's disease (AD)” with reference to an allele means that an individual who possesses the allele without detectable symptoms of AD is more likely to develop or have AD than someone who does not have the allele. The allele may be over-represented in frequency in individuals with AD as compared to individuals who do not have AD. Thus, the allele can be used to predict disease even in pre-symptomatic or pre-diseased individuals.

As used herein, “indicative of the occurrence of Alzheimer's disease (AD)” with reference to an allele means that an individual who possesses the allele and manifests one or more symptoms of AD is more likely to have AD than someone who does not have the allele and either does or does not manifest one or more symptoms of AD. Thus, the allele may be used to diagnose AD and, in particular, differentially diagnose AD. For example, the allele may be used to distinguish an individual with AD-associated dementia from an individual with dementia resulting from a condition unrelated to AD. This is particularly of use in diagnosis of AD in individuals about age 50 or greater, about age 60 or greater or about age 65 or greater. In methods of using an allele to diagnose AD, determination of the presence or absence of the allele in an individual may be conducted in conjunction with other diagnostic tests for AD, including a variety of neuropsychological tests known to those of skill in the art and referred to herein.

As used herein, “information content” refers to the usefulness of a family to be informative for linkage. For a family to be informative for linkage, at least one of the parents must be doubly heterozygous. Thus, the informativeness of a marker is given by its heterozygosity (H), the fraction of individuals likely to be heterozygous at that locus, or the polymorphism information content (PIC), the fraction of matings in which a particular parent is expected to be fully informative.

As used herein, attributable risk refers to that percentage of a genetic disease, such as AD, that would disappear should an alteration a disease gene not exist in a population, for example, in humans.

As used herein, a combination refers to any association between or among two or more items. The combination can be two or more separate items, such as two compositions or two collections, can be a mixture thereof, such as a single mixture of the two or more items, or any variation thereof.

B. Genetics of Alzheimer's Disease (AD)

Alzheimer's disease (AD) is most commonly a late onset disease of the elderly occurring at about 50 years of age or older, and most typically at about 60 years of age or older. However, a small number of pedigrees have been described wherein an early onset form of the disease is inherited as an autosomal dominant trait with age-dependent penetrance. Early onset of AD occurs at ages less than 60 years of age, typically at less than about 50 years of age and most commonly between the years of about 30 to about 50 years of age. Genetic factors have been implicated in early and late onset AD.

The genetics of Alzheimer's disease is complex. Mutations in at least four genetic loci are associated with inherited susceptibility to AD (i.e., familial AD). Three genes have been associated with early onset AD: APP [β-amyloid precursor protein on chromosome 21; Goate et al. (1991) Nature 349:704-706; Chartier-Harlin et al. (1991) Nature 353:844-846; Murrell et al. (1991) Science 254:97-99; Karlinsky et al. (1992) Neurology 42:1445-1453], PS1 (presenilin 1) and PS2 (presenilin 2). Only one gene, the ε4 allele of the apolipoprotein E (APOE) gene on chromosome 19, has been associated with late onset AD. This gene was identified using both linkage analysis [Pericak-Vance et al. (1991) Am. J. Human Genet. 48:1034-1050] and association analysis [Corder et al. (1993) Science 261:921-923; Saunders et al. (1993) Neurology 43:1467-1472; Strittmatter et al. (1993) Proc. Natl. Acad. Sci. U.S.A. 90:1977-1981]. The magnitude of risk for AD conferred by the APOEε4 allele is age dependent, appearing maximal between 60 and 80 years of age [Tang et al. (1996) Am. J. Hum. Genet. 58:574-584; Frisoni et al. (1998) J. Neurol. Neurosurg. Psychiatry 65:103-106; Bickeboller et al. (1997) Am. J. Hum. Genet. 60:439-446]. The association of APOEε4 with AD appears to be strongest in individuals with an onset prior to 70 years of age [Farrer et al. (1997) J. Am. Med. Assoc. 278:1349-1356; Blacker et al. (1997) Neurology 48:139-147] and weakens with advanced age.

At least half of the people who develop late onset AD do not carry an APOEε4 allele. As much as 70% or more of the genetic variance for AD remains unaccounted for by APOEε4 and the three known early onset AD genes. Segregation analysis predicts a susceptibility locus accounting for more than 50% of onset age variation in AD, a considerably greater fraction than APOE [Daw et al. (2000) Am. J. Hum. Genet. 66:196-204]. Thus, the APOEε4 allele is neither necessary nor sufficient for AD; instead, it modulates the risk for development of AD [Corder et al. (1993) Science 261:921-923; Corder et al. (1994) Nature Genet. 7:180-184]. Additional genetic factors are therefore indicated for AD.

A number of other genes have been proposed to be risk factors for late-onset AD or to modify the association with APOEε4. These include the genes encoding butyrocholinesterase [Lehmann et al. (1997) Human Mol. Genet. 6:1933-1936], low-density lipoprotein receptor-related protein (LRP) [Kang et al. (1997) Neurology 49:56-61], very low-density lipoprotein receptor (VLDL) [Okuizumi et al. (1995) Nature Genet. 11:207-209], cystatin C(CST3) [see Nitsch et al. (1999) Soc. Neuroscience 25 (Part 1):432.11, Abstracts for the 29th Annual Meeting, Oct. 23-28, 1999, Miami Beach, Fla. and Crawford et al. (2000) Neurobiol. Aging 21 (Suppl.):S204, Abstract 927, Abstracts for the World Alzheimer Congress 2000, Jul. 9-18, 2000, Washington, D.C.] and α1-antichymotrypsin [Kamboch et al. (1995) Nature Genet. 10:486-488; Yoshiiwa et al. (1997) Ann. Neurol. 42:115-117]. None of these genes is located on chromosome 10.

Several full genome screens for AD susceptibility loci have been performed with varying results. For example, possible loci which have been proposed in linkage studies include one near the centromere of chromosome 12 (with chromosomes 4, 6 and 20 proposed as regions of interest for follow-up studies) [Pericak-Vance et al. (1997) JAMA 278:1237-1241 and Pericak-Vance et al. (1998) Neurobiol. Aging 19:S39-S42] and one on the proximal long arm of chromosome 19 [Pericak-Vance et al. (1991) Am. J. Hum. Genet. 48:1034-1050]. The results of linkage analyses have been reported to suggest linkage of AD on chromosomes 1, 5, 9, 10, 12, 14, 19 and 21 [Kehoe et al. (1999) Hum. Mol. Genet. 8:237-245]. In the study reported by Kehoe et al. [(1999) Hum. Mol. Genet. 8:237-245], 292 affected sibling pairs with AD and onset age of ≧65 years were genotyped using 237 microsatellite markers separated by an average distance of 16.3 cM, and the data were analyzed by SPLINK [Holman and Clayton (1995) Am. J. Hum. Genet. 57:1221-1232] and MAPMAKER/SIBS [Lander and Krugylak (1995) Nature Genet. 11:241-247] for the whole sample as well as for samples stratified on the basis of whether both or neither members of an affected sibling pair possessed at least one APOE ε4 allele. Although multipoint LOD scores ≧1 were reported for markers on 12 different chromosomes in either the stratified or whole samples, only markers on chromosomes 1, 5, 9, 10 and 19 yielded multipoint LOD scores >1 in the whole sample. The linkage peak (LOD score of ˜2.27) on chromosome 10 observed in the analysis of the whole sample appears to have been located somewhere within the region of 50-70 cM. A maximum LOD score of 1.17 was reported for chromosome 10 in analysis of the stratified samples (i.e., the APOE ε4-negative sample) and appears to be provided by a marker located at about 100 cM.

In a follow-up of the study of Kehoe et al. [(1999) Hum. Mol. Genet. 8:237-245], additional markers were genotyped within each peak region to reduce the interval between each pair of markers to <5 cM [Myers et al. (2000) Neurobiol. Aging 21 (suppl.):S103]. The peak on chromosome 10 (reported to have yielded a LOD score of 1.87 in contrast to the LOD score of 2.27 reported by Kehoe et al. supra) reportedly increased to 3.11 with additional markers and samples and shifted in location towards the pter from D10S1211 (70.35 cM) to between D10S1227 and D10S1225 (peak at 60.42 cM). Significantly, this follow-up study reports that the initial peak previously found [as set forth in Kehoe et al. (supra)] in the APOE ε4-negative sample disappeared in this follow-up study using additional affected sibling pairs. Another genome scan for linkage of AD [Rogaeva et al. (2000) Neurobiol. Aging 21 (suppl.):S103] reports that markers on chromosome 10q23 (D10S1423-71 cM-D10S571) generated negative evidence for linkage.

The results of a genome-wide survey for AD risk loci using a case-control design were reported to indicate allelic association with AD at a marker (D10S1423) on chromosome 10p12-14 [Zubenko et al. (1998) Genomics 50:121-128], approximately 40 cM from the telomere. In a subsequent case-control study of marker D10S1423, a significant association of marker D10S1423 with AD was reported [Majores et al. (2000) Neurosci. Lett. 289:224-226].

C. Genetic Association with Alzheimer's Disease (AD) on Chromosome 10

Genetic analysis described herein led to the discovery of genetic association with AD on chromosome 10. The association identifies chromosome 10 as the location of one or more DNA segments or genes associated with AD and of considerable effect size. In particular, chromosome 10 is identified as containing at least one DNA segment or gene associated with AD and of comparable or greater impact than APOE, wherein the effect size of the gene is comparable to or greater than that of APOE.

As described herein (see EXAMPLE 3), a diallelic test of marker D10S583, located on chromosome 10q23.33, for association with AD using a family-based test method for association revealed significant association of an approximately 210-bp allele with protection against AD (nominal p=0.004, Bonferroni corrected p=0.04). Thus, D10S583 is in linkage disequilibrium with an allele that confers protection against AD. This finding indicates that there is an allele on chromosome 10 that confers in those who carry the allele protection against AD relative to those who do not carry the allele.

Furthermore, the finding of association of D10S583 with AD indicates that there is one or more AD DNA segments or AD genes on chromosome 10, and in particular chromosome 10q, that either directly cause or confer an increased susceptibility to AD (e.g., a “risk” or “disease” allele). A protective allele, such as the allele with which D10S583 is in linkage disequilibrium, generally has a counterpart disease risk allele. For example, the APOE gene, located in a peak linkage region on chromosome 19 identified in a genetic linkage analysis of late-onset AD families [Pericak-Vance et al. (1991) Am. J. Hum. Genet. 48:1034-1050], has three common alleles designated ε2, ε3 and ε4. The ε3 allele is the most common allele, and the ε2 and ε4 alleles are considered variants which affect genetic susceptibility to AD. The ε4 allele is associated with an increased risk and earlier age-at-onset whereas the ε2 allele confers a decreased risk and older age-at-onset [Corder et al. (1994) Nat. Genet. 7:180-184]. One or more AD genes on chromosome 10, and in particular chromosome 10q, may have multiple alleles, one of which is protective against AD and another which confers increased suceptibility to AD.

In addition, D10S583, as well as other markers on chromosome 10q, particularly markers on chromosome 10q22-q26, are linked to AD (see EXAMPLE 2). The peak linkage occurs on the distal approximately 70-85 cM of the q arm of chromosome 10, from about 85 cM extending distally to qter. In terms of the cytogenetic map of chromosome 10, the peak linkage extends from 10q22 to qter. The strongest linkage is on 10q23-q25. Linkage analysis reveals cosegregation of a marker with the disease trait within individual families, and thus provides evidence that within each family, a particular allele of a marker, such as an allele of D10S583, is relatively close to at least one DNA segment that causes AD or confers an increased susceptibility to AD. The discovery described herein of association between the AD-linked marker D10S583 and a population within families having affected members, be it association with affected family members or with unaffected members, reveals that there is at least one AD DNA segment or AD gene within linkage disequilibrium distance of D10S583 and that there are AD-associated marker alleles in the thus-defined region of chromosome 10 that may be used in determining a predisposition to or the occurence of AD in an individual.

Although the markers linked to AD as described in EXAMPLE 2, including D10S583, did not reveal a significant association with risk for AD, association analyses of multiple alleles of these markers revealed a trend toward risk. A disease gene, such as an AD gene, very likely may have several variant forms that place a person at risk for the disease, as well as variant forms that decrease the risk for AD and forms that are neutral (i.e., have a relative risk of 1.0). The data from association analyses of the linked markers described in EXAMPLE 2 are consistent with the possibility of multiple risk alleles of an AD gene on chromosome 10. Thus, the association of an allele of the AD-linked marker D10S583 with unaffected AD family members is consistent with the existence of at least one DNA segment that causes AD or confers increased susceptibility to AD on chromosome 10, and, in particular, chromosome 10q22-q26, such as the region of chromosome 10q23-q25, as well as being indicative of the presence of an allele on chromosome 10 that is protective against AD.

Any other marker found to be in linkage disequilibrium with D10S583 will be associated with an allele protective against AD and thus will also be evidence of the presence of at least one DNA segment that causes AD or confers increased susceptibility to AD on chromosome 10. Therefore, based on the discovery of association between D10S583 and AD, additional markers associated with AD or a protective allele may now be identified using methods as described herein and known in the art. The availabiltiy of additional markers is of particular interest in that it will increase the density of markers for this chromosomal region and can provide a basis for identification of an AD DNA segment or gene in the region of chromosome 10q, and in particular, chromosome 10q22-q26. An AD DNA segment or gene may be found in the vicinity of the marker or set of markers showing the highest correlation with AD. Furthermore, the availability of markers associated with AD makes possible genetic analysis-based methods of determining a predisposition to or the occurrence of AD in an individual by detection of a particular allele.

The discovery of one or more AD-associated genes on chromosome 10, as evidenced by significant association with AD on chromosome 10, thus provides the basis for genetic analysis methods described herein which include: methods for identifying polymorphisms linked to a DNA segment associated with AD; methods for detecting polymorphisms linked to a DNA segment associated with AD; methods for identifying polymorphisms associated with AD; methods for detecting polymorphisms associated with AD; methods for detecting the presence of a DNA segment associated with AD in a subject; methods for determining the level of risk for AD in a subject; methods for determining a predisposition to and/or the occurrence of AD in a subject; methods for identifying a region or regions of the human genome containing an AD DNA segment or AD gene; methods for predicting response to treatment for AD; methods for treating AD; methods for identifying a candidate AD DNA segment or AD gene; and methods for identifying an AD gene. Also provided herein are compositions that may be used in methods described herein, such as nucleic acids that may be used as probes or primers for detection of polymorphisms linked to AD-associated genes and combinations, kits and articles of manufacture containing the nucleic acids. Such compositions may also be used in methods of determining a predisposition to and/or the occurrence of AD in a subject.

D. Genetic Markers Associated with AD

Genetic markers associated with AD are provided herein. The markers are located on human chromosome 10. In particular embodiments of the genetic markers provided herein, the marker is located located on chromosome 10q22, 10q23, 10q24, 10q25 or 10q26. In further embodiments, the marker is located on chromosome 10q22, 10q23 or 10q24. In yet further embodiments, the marker is located on chromosome 10q23, 10q24 or 10q25. The markers can be over-represented in cases in case-control studies and/or can be associated with affected individuals in a family-based association analysis. Alternatively, the markers can be under-represented in cases in case-control studies and/or associated with unaffected individuals in a family-based association analysis. The markers can be identified through linkage disequilibrium or association assessment methods described herein or known to those of skill in the art, and provide scores or results indicative of linkage disequilibrium with an AD DNA segment or gene or of association with AD when tested by such assessment methods. The genetic markers are associated with AD as individual markers and/or in combinations, such as haplotypes, that are associated with AD.

Also provided herein are combinations of genetic markers which are associated with AD. In one embodiment, each genetic marker in a combination is associated with AD. In other embodiments, some of the genetic markers in the combination are associated with AD and some of the genetic markers are not or none of the genetic markers is associated with AD. In such embodiments, the combination of markers as a whole is associated with AD, such as in the case of a haplotype.

1. Genetic Markers on Human Chromosome 10

a. Genetic Markers

A genetic marker is a DNA segment with an identifiable location in a chromosome. Genetic markers may be used in a variety of genetic studies such as, for example, locating the chromosomal position or locus of a DNA sequence of interest, and determining if a subject is predisposed to or has a particular disease.

Because DNA sequences that are relatively close together on a chromosome tend to be inherited together, tracking of a genetic marker through generations in a family and comparing its inheritance to the inheritance of another DNA sequence of interest can provide information useful in determining the relative position of the DNA sequence of interest on a chromosome. Genetic markers particularly useful in such genetic studies are polymorphic. Such markers also may have an adequate level of heterozygosity to allow a reasonable probability that a randomly selected person will be heterozygous.

The occurrence of variant forms of a particular DNA sequence, e.g., a gene, is referred to as polymorphism. A region of a DNA segment in which variation occurs may be referred to as a polymorphic region or site. A polymorphic region can be a single nucleotide (single nucleotide polymorphism or SNP), the identity of which differs, e.g., in different alleles, or can be two or more nucleotides in length. For example, variant forms of a DNA sequence may differ by an insertion or deletion of one or more nucleotides, insertion of a sequence that was duplicated, inversion of a sequence or conversion of a single nucleotide to a different nucleotide. Each individual can carry two different forms of the specific sequence or two identical forms of the sequence. More than two forms of a polymorphism may exist for a specific DNA marker in the population, but in one family just four forms are possible: two from each parent. Each child inherits one form of the polymorphism from each parent. Thus, the origin of each chromosome can be traced (maternal or paternal origin).

Differences between polymorphic forms of a specific DNA sequence may be detected in a variety of ways. For example, if the polymorphism is such that it creates or deletes a restriction enzyme site, such differences may be traced by using restriction enzymes that recognize specific DNA sequences. Restriction enzymes cut (digest) DNA at sites in their specific recognized sequence, resulting in a collection of fragments of the DNA. When a change exists in a DNA sequence that alters a sequence recognized by a restriction enzyme to one not recognized, the fragments of DNA produced by restriction enzyme digestion of the region will be of different sizes. The various possible fragment sizes from a given region therefore depend on the precise sequence of DNA in the region. Variation in the fragments produced is termed “restriction fragment length polymorphism” (RFLP). The different sized-fragments reflecting variant DNA sequences can be visualized by separating the digested DNA according to its size on an agarose gel and visualizing the individual fragments by annealing to a labeled, e.g., radioactively or otherwise labeled, DNA “probe”. RFLPs occur on average every 10 kb.

RFLPs may be somewhat limiting in genetic analyses in that they usually give only two alleles at a locus and not all parents are heterozygous for these alleles and thus informative for linkage [see, e.g., Botstein et al. (1980) Am. J. Hum. Genet. 32:314-331]. Newer analytic methods take advantage of the presence of DNA sequences that are repeated in tandem, for a variable number of repeats, and that are scattered throughout the human genome. The first of these described were variable number tandem repeats of core sequences (VNTRs) [Jeffreys et al. (1985) Nature 314:67-73; Nakamura et al. (1987) Science 235:1616-1622; Weber (1989) Am. J. Hum. Genet. 44:388-396]. VNTRs may be detected using unique sequences of DNA adjacent to the tandem repeat as marker probes, and digesting the DNA with restriction enzymes that do not recognize sites within the core sequence. VNTRs may also be detected using nucleic acid amplification methods. Highly informative VNTR loci have not been found on all chromosome arms, and those which have been identified are often situated near telomeres [Royle et al. (1998) Genomics 3:352-360], leaving regions of the genome out of reach of these multiallelic marker loci.

Eukaryotic DNA has tandem repeats of very short simple sequences termed SSRs (simple sequence repeat polymorphisms) such as, for example, (dC-dA)ₙ or (dG-dT)ₙ where n=10-60 (termed GT repeat). These are also referred to as short tandem repeat polymorphisms (STRPs) and microsatellite markers. The (dG-dT) repeats occur every 30-60 kb along the genome [Weber et al. (1989) Am. J. Hum. Genet. 44:388-396; Litt et al. (1989) Am. J. Hum. Genet. 44:397-401], and Alu 3′ (A)n repeats occur approximately every 5 kb [Economou (1990) Proc. Natl. Acad. Sci. U.S.A. 87:2951-4]. Repeat polymorphisms include dinucleotide, trinucleotide and tetranucleotide repeats. Dinucleotide repeats are informative and fairly prevalent in the genome. The small size of the repeat brings about diversity of its allele sizes and thus there is a greater chance that any one person will be heterozygous for the marker. Trinucleotide and tetranucleotide repeats are repeats of three and four nucleotides.

Oligonucleotides corresponding to flanking regions of these repeats may be used as primers for the polymerase chain reaction (PCR) [Saiki (1988) Science 239:484-491] on a small sample of DNA. By amplifying the DNA with labeled, e.g., radioactive or fluorescent, nucleotides, the sample may be quickly resolved on a sequencing gel and visualized by known methods, e.g., autoradiography or fluorescence detection. Because these polymorphisms are comprised of alleles that may differ in length by only a few base pairs, they generally are not detectable by conventional Southern blotting as used in traditional RFLP analysis. The use of PCR to characterize SSRs such as GT polymorphic markers enables the use of less DNA, typically only ten nanograms of genomic DNA is needed, and is faster than standard RFLP analysis, because it essentially only involves amplification and electrophoresis.

Microsatellites have been used extensively in linkage analysis (see, e.g., http://carbon.wi.mit.edu:8000/cgi-bin/contig/phys_map; http://www.chlc.org/; http://gdb.infobiogen.fr/gdb/contact.html#baltimore). They have many alleles and therefore are highly informative. Although microsatellites may be used in fine mapping and association analysis, they may have one or more features that should be considered in connection with such use. For example, the large number of alleles may become a consideration when using haplotype-based methods, they are not usually intragenic, and they may have relatively high and variable mutation rates which may affect linkage disequilibrium between a marker and disease mutation.

SNP markers may also be used in fine mapping and association analysis, as well as linkage analysis [see, e.g., Kruglyak (1997) Nature Genetics 17:21-24]. Although an SNP may have limited information content, combinations of SNPs (which individually occur about every 100-300 bases) may yield informative haplotypes. SNP databases are available (see, e.g., http://www.ibc.wustl.edu/SNP/; http://www.ncbi.nlm.nih.gov/SNP/; http://www. genome.wi.mit.edu/SNP/human/index.html). Assay systems for determining SNPs include synthetic nucleotide arrays to which labeled, amplified DNA is hybridized [see, e.g., Lipshutz et al. (1999) Nature Genet. 21:2-24; single base primer extension methods [Pastinen et al. (1997) Genome Res. 7:606-614], mass spectroscopy on tagged beads, and solution assays in which allele-specific oligonucleotides are cleaved or joined at the position of the SNP allele, resulting in activation of a fluorescent reporter system [see, e.g., Landegren et al. (1998) Genome Res. 8:769-776].

b. Chromosome 10

The estimated physical length of chromosome 10 is about 144 Mb. The genetic or linkage map of chromosome 10, generated based upon frequency of recombination between genomic segments, estimates that chromosome 10 is about 175 centiMorgans (cM) in length. Cytogenetic maps of chromosome 10 distinguish regions of the chromosome as p or q bands. The bands refer to bands observed on the Giemsa-stained chromosome. Approximate locations for the bands have been determined. For example, band 10q21.3, the most distal of the 10q21 bands, begins at about 65 Mb and ends at about 70 Mb on chromosome 10. Band 10q22.1, the most proximal of the 10q22 bands, begins at about 70 Mb (or about 86-92 cM) and ends at about 77 Mb on chromosome 10. Band 10q23.1, the most proximal of the 10q23 bands, begins at about 85 Mb and ends at about 90 Mb. Band 10q23.33, the most distal of the 10q23 bands, begins at about 99 Mb and ends at about 103 Mb. Band 10q25.1, the most proximal of the 10q25 bands, begins at about 112 Mb and ends at about 117 Mb. Band 10q25.3, the most distal of the 10q25 bands, begins at about 119 Mb and ends at about 123 Mb. Band 10q26.11, the most proximal of the 10q26 bands, begins at about 123 Mb and ends at about 125 Mb.

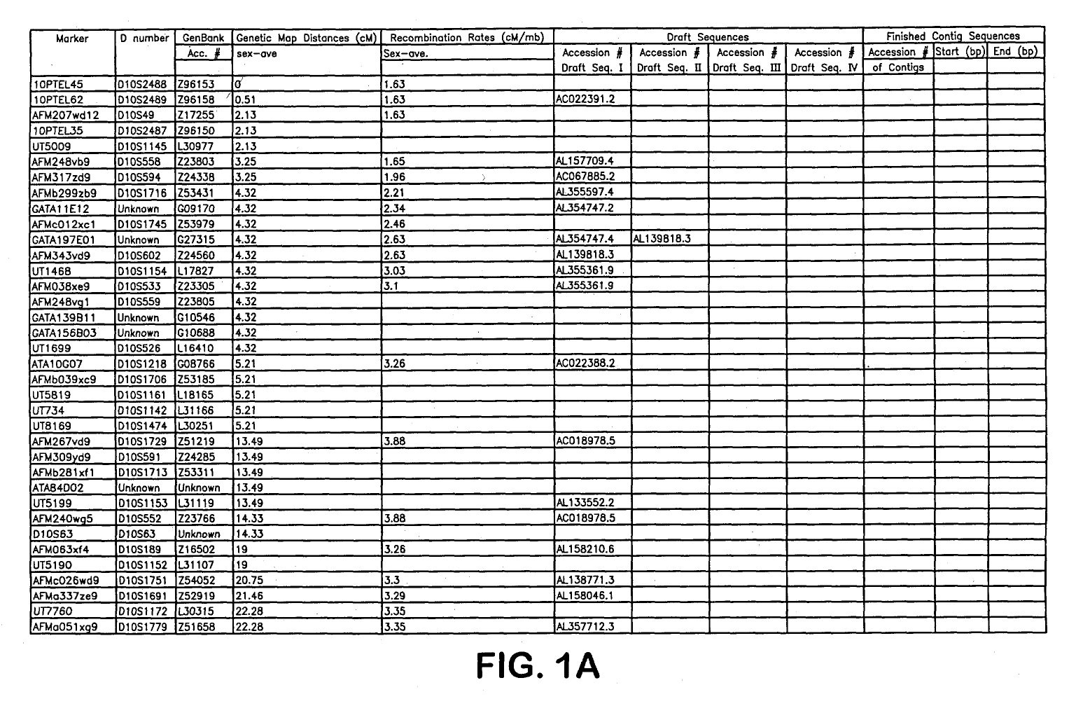

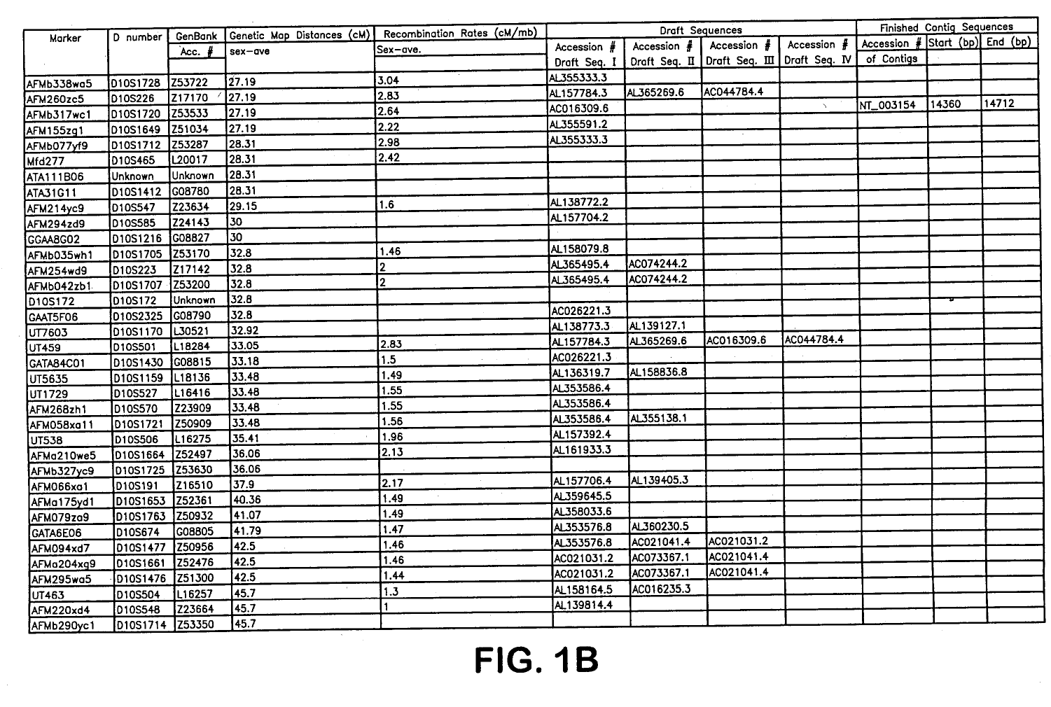

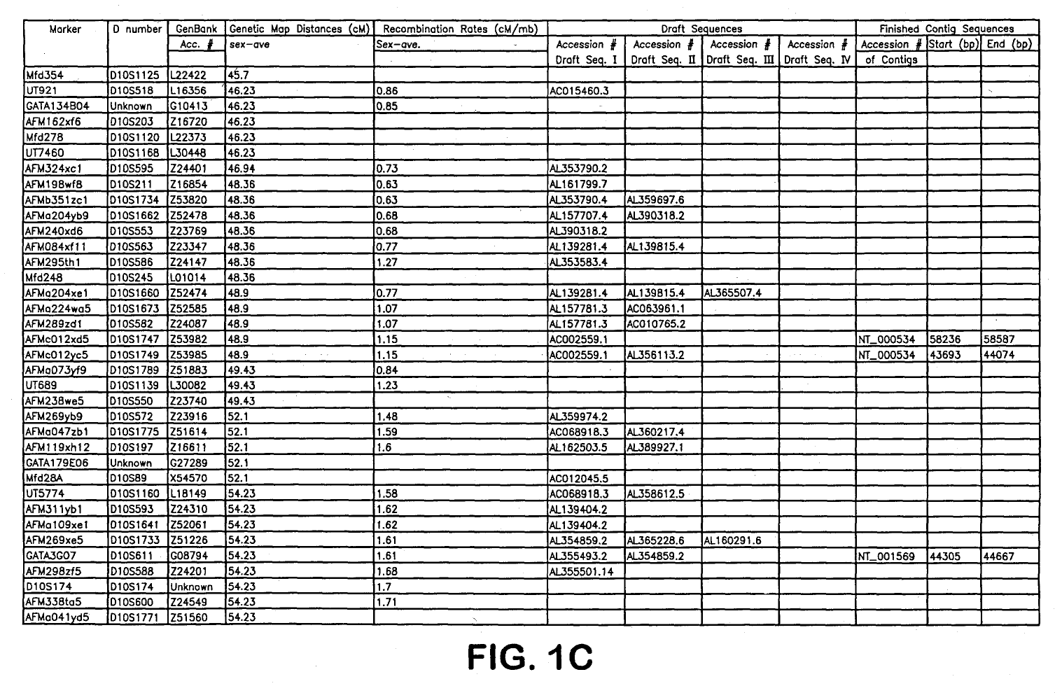

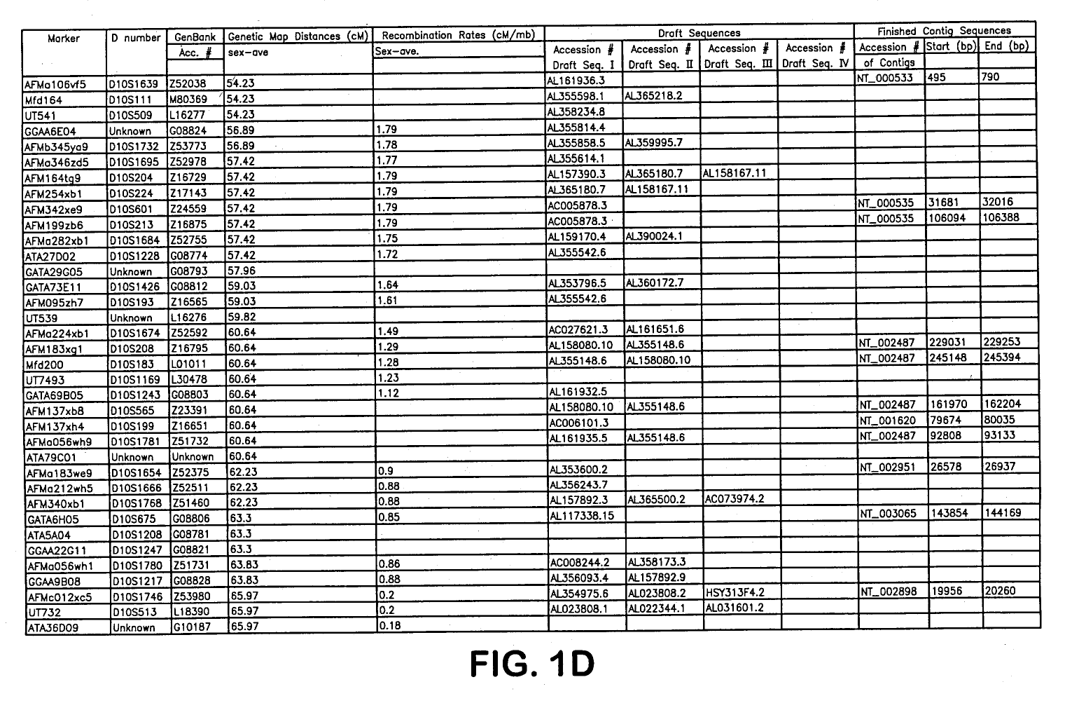

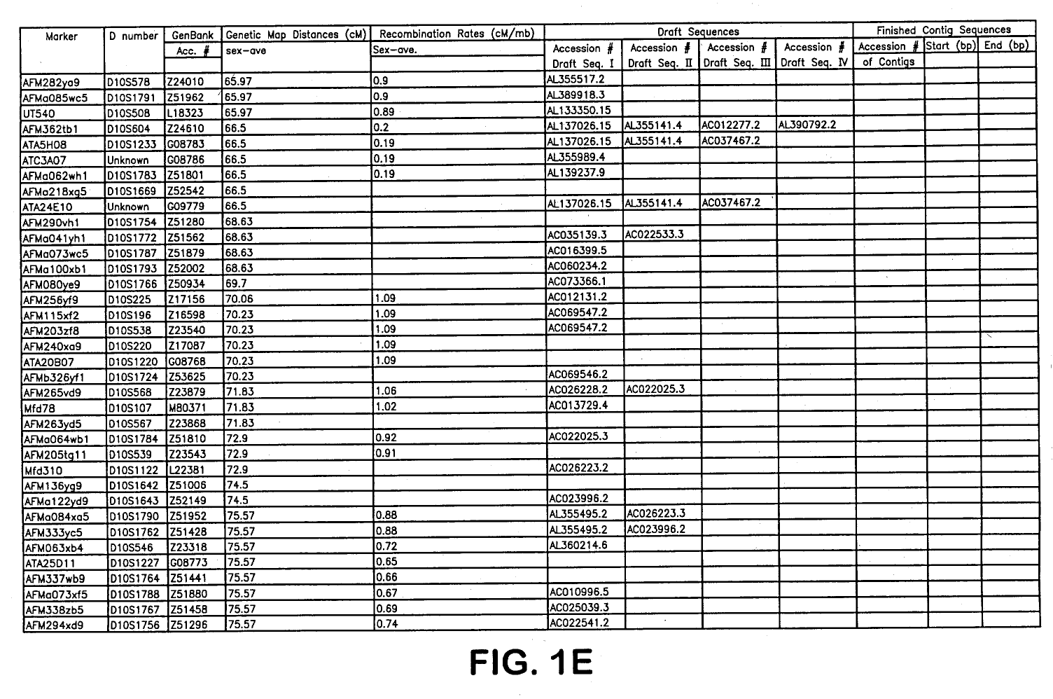

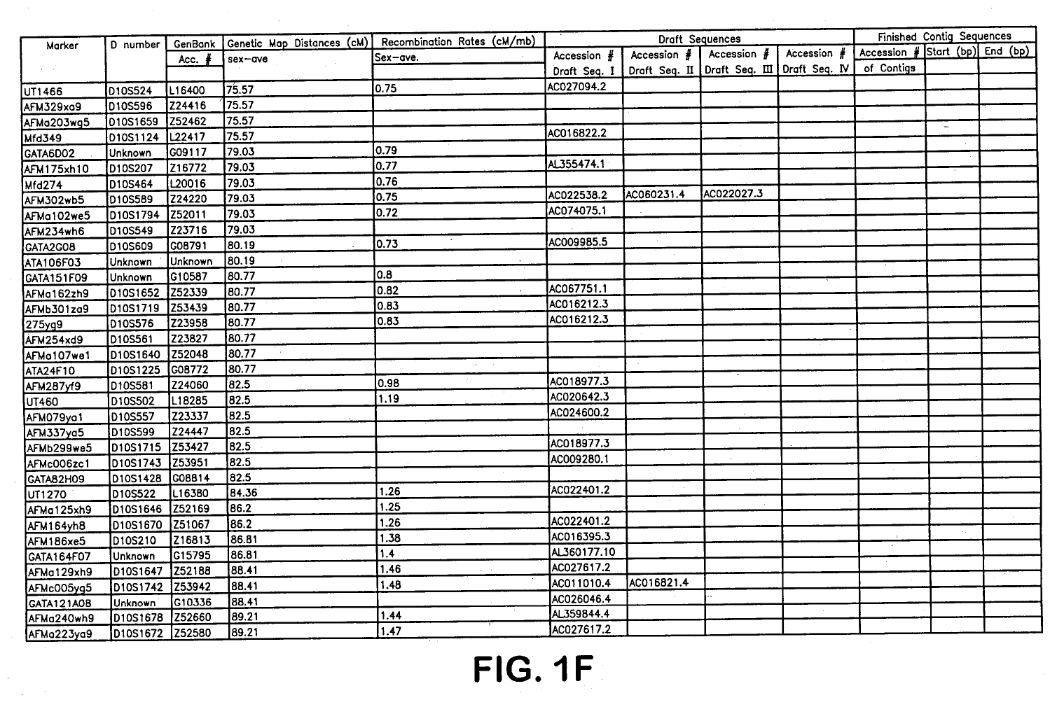

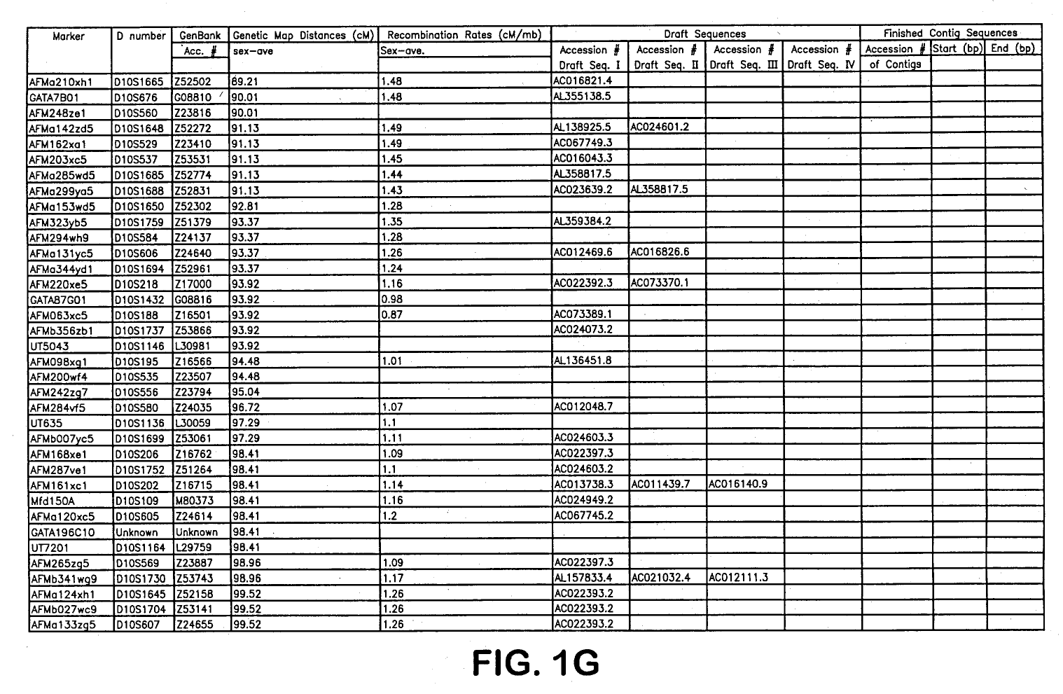

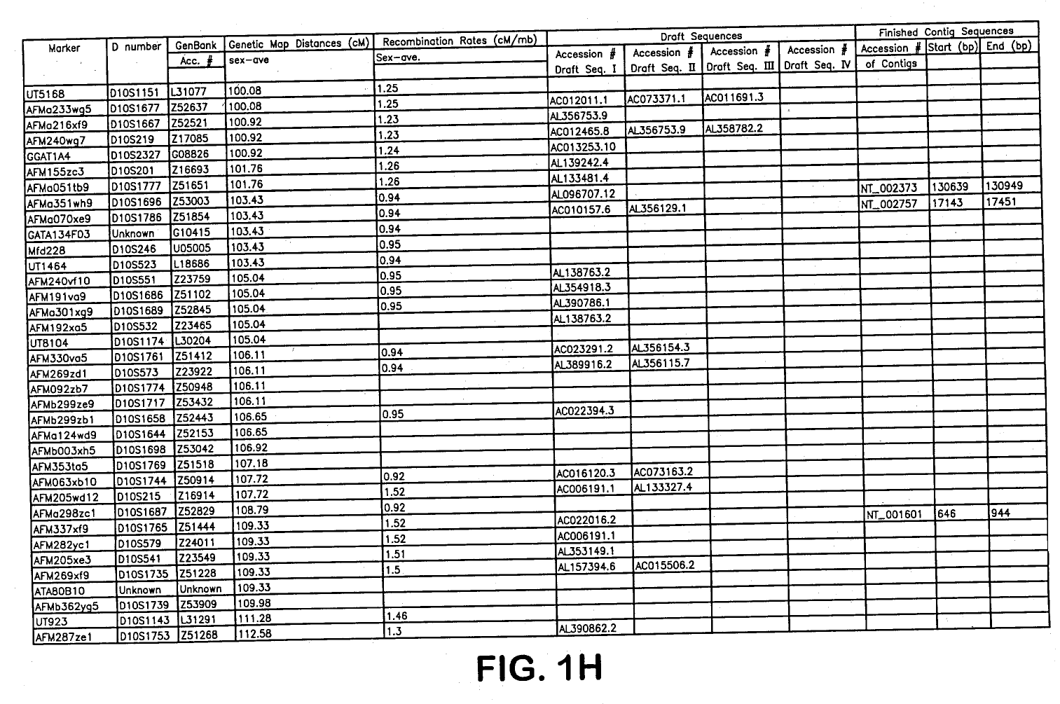

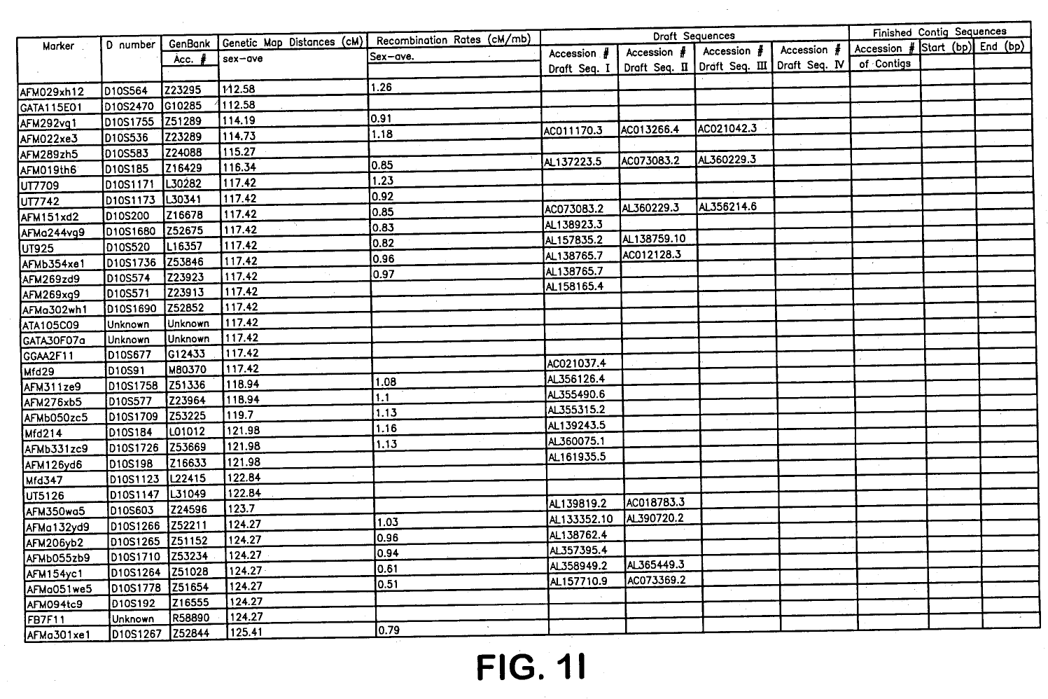

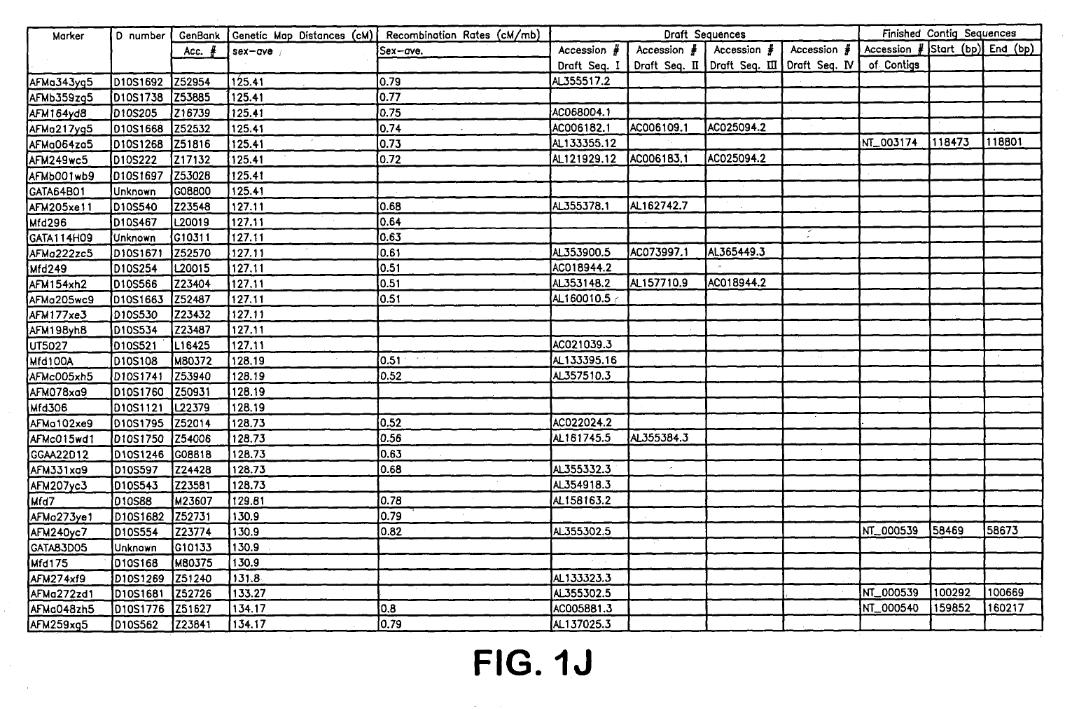

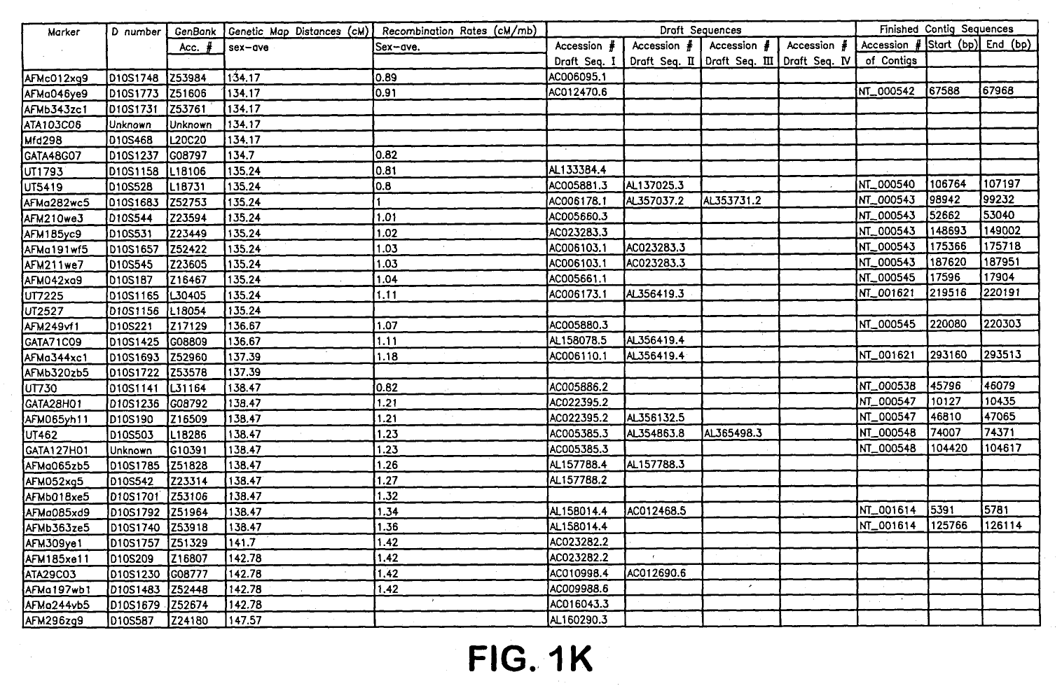

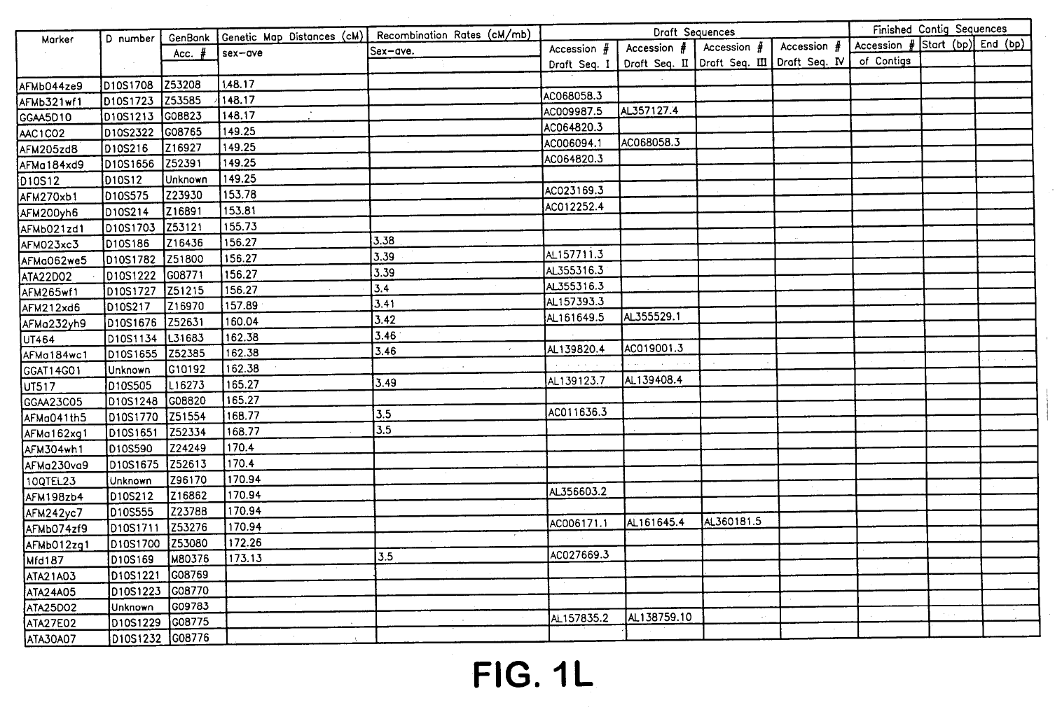

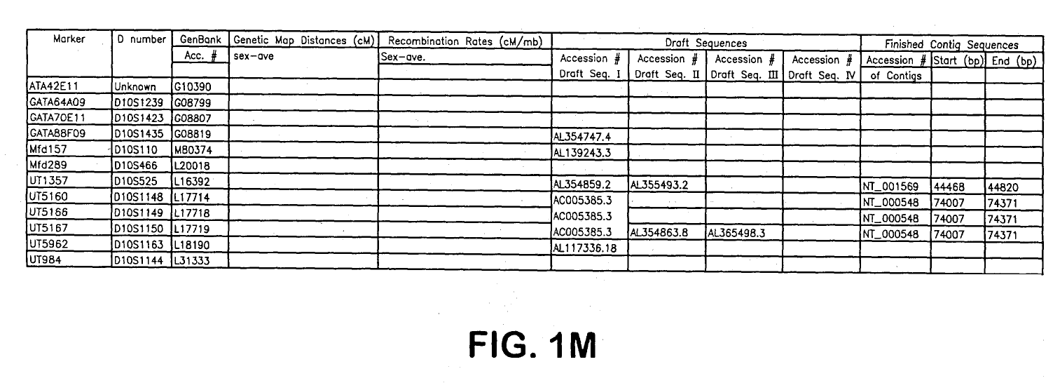

More than 400 short tandem repeat polymorphisms which serve as genetic markers have been mapped on chromosome 10. FIG. 1 lists many of these markers and provides the sex-averaged genetic map distance for each marker as reported by the Marshfield Center for Medical Genetics [see Yu et al. (2001) Nature 409:951-953 and (http://research.marshfieldclinic.org/genetics/)]. Also provided in FIG. 1 are references to GenBank, Human Genome Draft sequence and Contig sequence accession numbers for many of these markers.

Included among the polymorphic markers in FIG. 1 is marker D10S583 discovered, as described herein, to be associated with protection against AD. Also included among the markers listed in FIG. 1 are markers linked to AD, including D10S583. As described in the EXAMPLES, markers D10S583, D10S1710, D10S566, D10S1671 and D10S1741 each yielded LOD scores >1 for the whole sample in at least one of several AD linkage analyses. These markers are dinucleotide repeat sequences.

2. Genetic Association

When two loci are extremely close together, recombination between them is very rare, and the rate at which the two neighboring loci recombine can be so slow as to be unobservable except over many generations. The resulting allelic association is generally referred to as linkage disequilibrium. Linkage disequilibrium can be defined as specific alleles at two or more loci that are observed together on a chromosome more often than expected from their frequencies in the population. As a consequence of linkage disequilibrium, the frequency of all other alleles present in a haplotype carrying a trait-causing allele will also be increased (just as the trait-causing allele is increased in an affected, or trait-positive, population) compared to the frequency in a trait-negative or random control population. Therefore, association between the trait and any allele in linkage disequilibrium with the trait-causing allele will suffice to suggest the presence of a trait-related DNA segment in that particular region of a chromosome. On this basis, association studies are used in methods of locating and discovering disease-susceptibility genes.

A marker locus must be tightly linked to the disease locus in order for linkage disequilibrium to exist between the loci. In particular, loci must be very close in order to have appreciable linkage disequilibrium that may be useful for association studies. Association studies rely on the retention of adjacent DNA variants over many generations in historic ancestries, and, thus, disease-associated regions are theoretically small in outbred random mating populations. In practice, however, it is common to find some degree of linkage disequilibrium between alleles that are up to about 1 to 2 cM apart, or even 3 to 4 cM apart, and this can be used for disease gene mapping [Jorde (1995) Am. J. Hum. Genet. 56:11-14; Xiong and Guo (1997) Am. J. Hum. Genet. 60:1513-1531]. In contrast, linkage studies, by relying on identification of haplotypes that are inherited intact over several generations (such as in families or pedigrees of known ancestry) focus on recent, usually observable ancestry in which there have been relatively few opportunities for recombination to occur. Thus, disease gene regions identified by linkage will often be large, encompassing many tens of megabases of DNA.

The power of genetic association analysis to detect genetic contributions to complex disease can be much greater than that of linkage studies. Linkage analysis can be limited by a lack of power to exclude regions or to detect loci with modest effects. Association tests can be capable of detecting loci with smaller effects [Risch and Merikangas (1996) Science 273:1516-1517] which may not be detectable by linkage analysis.

The aim of association studies when used to discover disease-susceptibility genes is to identify particular genetic variants that correlate with the disease phenotype at the population level. Association at the population level may be used in the process of identifying a disease-susceptibility gene or DNA segment because it provides an indication that a particular marker is either a functional variant underlying the disease (i.e., a polymorphism that is directly involved in causing a particular trait) or is extremely close to the disease gene on a chromosome. When a marker analyzed for association with a disease is a functional variant, association is the result of the direct effect of the genotype on the phenotypic outcome. When a marker being analyzed for association is an anonymous marker, the occurrence of association is the result of linkage disequilibrium between the marker and a functional variant.

There are a number of methods typically used in assessing genetic association as an indication of linkage disequilibrium, including the epidemiological case-control study of unrelated subjects and methods using family-based controls. Although the case-control design is relatively simple, it is the most prone to identifying DNA variants that prove to be spuriously associated (i.e., association without linkage) with disease [Cardon and Bell (2001) Nature Rev. Genet. 2:91-99]. Spurious association can be due to the structure of the population studied rather than to linkage disequilibrium. Thus, for example, if cases and controls are not ethincally comparable, then differences in allele frequency can emerge at loci that differentiate the groups whether the alleles are causally related to disease or not (a phenomenon referred to as population stratification). Linkage analysis of such spuriously associated allelic variants, however, would not detect evidence of significant linkage because there would be no familial segregation of the variants. Therefore, putative association between a marker allele and a disease trait identified in a case-control study should be tested for evidence of linkage between the marker and the disease before a conclusion of probable linkage disequilibrium is made. Association tests that avoid some of the problems of the standard case-control study utilize family-based controls in which parental alleles or haplotypes not transmitted to affected offspring are used as controls.

Although genetic linkage between a marker and a disease, such as AD, demonstrates a true genetic basis for the disease and indicates proximity between the marker and a disease locus which has use in mapping the location of a disease locus on a chromosome, it is determined through analysis of transmission of marker alleles through individual families. The specific alleles segregating together in one family in a linkage study may well differ from alleles at the same loci segregating together in another family. The identities of the differing alleles of a marker that may cosegregate with a disease trait in different families are not the relevant results in the analysis of linkage. Instead, the result of import in linkage analysis is the finding that any allele of a marker cosegregates with a disease trait in separate affected families. Linkage is a property of the relative position loci, not their alleles, and the observed cosegregation within a given family can involve any allele at the marker locus.

Linked markers that are not in linkage disequilibrium with a DNA segment associated with a disease may be used in determining a predisposition to or the occurrence of a genetic disease in an individual having an affected relative of known genotype with respect to the marker. Thus, the allele of the polymorphic marker detected in such methods of determining a predisposition to or the occurrence of a disease must be in phase with a disease allele. Phase can be established by determining the presence or absence of an individual's allele for a marker in two relatives (such as relatives of the first or second degree, and in particular, parents) of the individual who are of known phenotype with respect to the disease. For an individual's allele to be in phase, at least one of the relatives should have the disease and should also be heterozygous for the allele. Strategies for setting phase in different families are known [see, e.g., Lazarou (1993) Clin. Genet. 43:150-156]. Within an affected family, the allele of a marker that is in phase with a disease allele is usually the same in each affected member of the family because there is a low probability of recombination between the two loci. However, in general, the phase relationship between an allele of a polymorphic marker and disease allele is different in each family. If a particular allele of a linked marker happens to be in linkage disequilibrium with the disease allele, that same allele is likely to be in phase with the disease allele in any family. Methods for determining a predisposition to or the occurrence of a disease using a linked marker rely on the availability of phenotype and genotype information of relatives, at least one of whom has the disease. For late-onset diseases, such information may not always be available.

In contrast to genetic linkage, which is a property of loci, genetic association is a property of alleles. Association analysis involves a determiniation of a correlation between a single, specific allele and a trait across a population, not only within individual families. Thus, a particular allele found through an association study to be in linkage disequilibrium with a disease allele can form the basis of a method of determining a predisposition to or the occurrence of the disease in any individual. Such methods would not involve a determination of phase of an allele and thus would not be limited in terms of the individuals who may be screened in the method.

3. Analysis of Genetic Association

Association studies explore the relationships among frequencies for sets of alleles between loci. Association studies may be conducted within the general population and are not limited to studies performed on related individuals in affected families. There are several methods for testing for association.

a. Case-Control Studies

The simplest form of association analysis is the case-control study in which unrelated populations of affected (or trait-positive) subjects (i.e., case individuals) and unrelated control (unaffected, trait-negative or random) individuals are analyzed. Such population-based association studies do not concern familial inheritance but compare the prevalence of a particular genetic marker or set of markers in case-control populations. Marker allele frequencies in each population may be compared using a chi-squared or Fisher's exact test (see, e.g., linkage.rockefeller.edu/software/utilities).

The control group is typically “matched” as much as possible to the case population, particularly to avoid problems of population stratification. Thus, the control group may be ethnically matched to the case population and matched for the main known confusion factor for the trait under study (e.g., age-matched for an age-dependent trait). An important step in the dissection of complex traits using association studies is the choice of case-control populations [see Lander and Schork (1994) Science 265:2037-2048]. A major step in the choice of case-control populations is the clinical definition of a given trait or phenotype. A genetic trait may be analyzed by association methods by carefully selecting the individuals to be included in the trait-positive and trait-negative phenotypic groups. Several criteria are often useful: clinical phenotype, age at onset, family history and severity. The selection procedure for continuous or quantitative traits (such as blood pressure, for example) involves selecting individuals at opposite ends of the phenotype distribution of the trait under study, so as to include in these trait-positive and trait-negative populations individuals with non-overlapping phenotypes. Preferably, case-control populations contain phenotypically homogeneous populations. Trait-positive and trait-negative populations contain phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with markers. The selection of those drastically different but relatively uniform phenotypes enables efficient comparisons in association studies and the possible detection of marked differences at the genetic level, provided that the sample sizes of the populations under study are significant enough.

Allelic frequencies of markers in populations can be determined by genotyping pooled DNA samples or individual samples. When each individual is genotyped separately, simple gene counting may be applied to determine the frequency of an allele or of a genotype in a given population. The proportional representation of the allele for the population is then determined.

The allelic frequencies of the marker in case and control populations are analyzed to determine whether a statistically significant association exists between the genotype and phenotype. The statistical significance of a correlation between phenotype and genotype may be determined by any statistical test known in the art and with any accepted threshold of statistical significance being required. The application of particular methods and thresholds of significance are well within the level of skill of one skilled in the art. A commonly used statistical test is a chi-square test with one degree of freedom. A P-value is calculated, which is the probability that a statistic as large or larger than the observed one would occur by chance. If a statistically significant association with a trait is identified for at least one or more of the analyzed markers, it may be assumed that either the associated allele is directly responsible for causing the trait (i.e., the associated allele is the trait-causing allele), or more likely the associated allele is in linkage disequilibrium with the trait-causing allele.

In testing the association of a particular allele against the disease phenotype, it may be useful to correct the results. One such correction method is referred to as the “Bonferroni” correction in which the probability value required to give significance is divided by the number of tests conducted. For example, if five markers are tested, each with five alleles, a probability value of 0.002 would be required to declare significance at the 5% level.

Case-control studies can be susceptible to false positive (type I) and false negative (type II) errors. Thus, a negative result may mean a lack of association or a false negative due to insufficient power to detect association. A positive result may mean an allelic association with disease, the presence of an unknown factor such as population stratification between cases and controls or a false positive due to an insufficient sample size for the tests being conducted. Calculators (see, e.g., http://www.stat.ucla.edu/calculators/powercalc/binomial/case-control/b-case-control-samp.html) are available to estimate required sample size for a given marker frequency, relative risk of interest, power and significance level (corrected if necessary for multiple tests).

Typical association studies based on candidate genes, and in particular, case-control studies, may have a limited ability to discern true medium-sized signals from false positives [see, e.g., Emahazion et al. (2001) Trends Genet. 17:407-413]. Thus, reports of positive association findings frequently cannot be replicated. For example, an initial report of a positive association of the GG-genotype of the TNFRSF6 gene with early-onset AD obtained in an unrelated case (Scottish sporadic early-onset AD sample)-control study [Feuk et al. (2000) Hum. Genet. 107:391-396] was not replicated in a subsequent case (Dutch population early-onset AD sample)-control study [Theuns et al. (2001) Hum. Genet. 108:552-553]. A case (Swedish patients with sporadic late-onset AD)-control study of 15 polymorphisms in candidate genes for sporadic AD that previously had been reported to have significant association with AD found no strong evidence of association for any of the loci in the Swedish population [Prince et al. (2001) Eur. J. Hum. Genet. 9:437-444].

b. Case-Control Studies Using Family-Based Controls

Case-control studies using family-based controls have been developed to address possible errors relating to inadequate matching of unrelated cases and controls. Unlike case-control tests, family-based tests are not affected by population stratification, which can lead to spurious associations of a marker allele with disease susceptibility. Such analytical techniques include the transmission disequilibrium test (TDT) [Spielman et al. (1993) Am. J. Hum. Genet. 52:506-516], affected family based control test (AFBAC) [Thomson (1995) Am. J. Hum. Genet. 57:487-498 and Schaid and Sommer (1994) Am. J. Hum. Genet. 55:402-409] and the haplotype relative risk test (HRR) [Falk and Rubinstein (1987) Ann. Hum. Genet. 51:227-233; Terwilliger and Ott (1992) Hum. Hered. 42:337-346]. In these methods, family members (usually unaffected) can be used as internal controls. In the HRR and AFBAC tests, an affected individual and two parents are typed for a marker hypothesized to have an allele associated with the disease. The number of control alleles are derived from the parental alleles not transmitted to the affected child, and these are compared to the number of alleles transmitted to the affected child by a chi-squared test. In the TDT test, one of the parents must be heterozygous for the marker concerned, and the comparison is made between the alleles that are transmitted to the affected child and those that are not. Deviation from the expected Mendelian 50% transmission is tested by a chi-squared or Fisher's exact test.

The TDT focuses on alleles transmitted to affected offspring, but is formulated to take account of both the linkage and the disequilibrium that underlie the association. Depending on the data structure, TDTs are tests of either linkage or linkage and association. The proposed test statistic is a McNemar's chi-square and tests the null hypothesis that the putative disease-associated alleles transmitted 50% of the time from heterozygous parents; under the alternative hypothesis, the disease-associated allele will be transmitted more often.

The TDT has been extended to take into account multiallelic marker loci [Spielman and Ewens (1996) Am. J. Hum. Genet. 59:983-989; Sham and Curtis (1995) Ann. Hum. Genet. 59:323-336; Bickeboeller and Clerget-Darpoux (1995) Genet. Epidemiol. 12:865-870; and Rice et al. (1995) Genet. Epidemiol. 12:659-664], the availability of only one parent [Sun et al. (1999) Am. J. Epidemiol. 150:97-104], analysis of affected sibs or trios [Martin et al. (1997) Am. J. Hum. Genet. 61:439-448], multiple analysis of linked alleles in haplotypes [Clayton and Jones (1999) Am. J. Hum. Genet. 65:1161-1169 and Clayton (1999) Am. J. Hum. Genet. 65:1170-1177], pooled genotyping of affected children [Risch and Teng (1998) Genome Res. 8:1273-1288] and transmission from parents homozygous at a tightly linked locus [Lie et al. (1999) Am. J. Hum. Genet. 64:793-800]. Family-based tests, such as TDT, have largely required knowledge of parental marker genotypes; however, for late-onset diseases, parental data are often not available. There are tests of linkage and association that use unaffected siblings as surrogates for untyped parents from which probable parental genotypes may be derived [Spielman and Ewens (1998) Am. J. Hum. Genet. 62:450-458 (also referred to as the sib-TDT or S-TDT); Horvath and Laird (1998) Am. J. Hum. Genet. 63:1886-1897; Boehnke and Langefeld (1998) Am. J. Hum. Genet. 62:950-961]. The discordant unaffected sibling provides information on the alleles not segregating to affected individuals.

The FBAT is a unified approach to family-based association testing that is similar in design to the TDT but can accomodate variations in pedigree structures, arbitrary missing genotype information and various different disease models [Rabinowitz and Laird (2000) Hum. Hered. 50:227-233; Laird et al. (2000) Genet. Epi. 19 (Suppl. 1):S36-S42]. To account for the presence of linkage when using multiple affected siblings per nuclear family, the FBAT allows robust variance estimation (referred to as EV-FBAT) based on the empirical variance-covariance matrix of the contributions of each family to the score statistic [Lake et al. (2000) Am. J. Hum. Genet. 67:1515-1525].

A number of computer software programs are available for statistical analysis of genotyping data in family-based association tests, including the FBAT program [Rabinowitz and Laird (2000) Hum. Hered. 50:211-223; see also http://www.biostat.harvard.edu/fbat/default.html], the GASSOC program of statistical methods including an extension of the TDT for multiple marker alleles [Schaid (1996) Genet. Epidemiol. 13:423-449; see also http://www.mayo.edu.statgen/gassoc], the Quantitative (Trait) Transmission/Disequilibrium Test (QTDT) which includes support for the methods of Abecasis et al. [(2000) Am. J. Hum. Genet. 66:279-292], Fulker et al. [(1999) Am. J. Hum. Genet. 64:259-267], Monks et al. [(1998) Am. J. Hum. Genet. 63:1507-1516], Allison [(1997) Am. J. Hum. Genet. 60:676-690; TDTQ5] and Rabinowitz [(1997) Hum. Hered. 47:342-350] [see also http://www.well.ox.ac.uk/asthma/QTDT], the Transmission Disequilibrium Test and SIB Transmission Disequilibrium Test (TDT/S-TDT) [Spielman et al. (1993) Am. J. Hum. Genet. 52:506-516 and Spielman and Ewens (1998) Am. J. Hum. Genet. 62:450-458; see also http://spielman07.med.upenn.edu/TDT.htm], the ASSOC program in the Statistical Analysis for Genetic Epidemiology (SAGE) program uses the method of George and Elston [(1987) Genet. Epidemiol. 4:193-201; see also http://darwin.cwru.edu/pub/sage.html] and TRANSMIT [see http://www.mrc-bsu.cam.ac.uk/pub/methodology/genetics/].

4. Genetic Markers Associated with AD on Chromosome 10

As shown by the results of association analyses of genotyping data with respect to marker D10S583 located on chromosome 10 (see EXAMPLE 3), provided herein are genetic markers on chromosome 10 which have an allele that is associated with AD. Included within the markers provided herein are markers located in the region of 10q22, 10q23, 10q24, 10q25 and/or 10q26. In particular, markers located in the region of 10q22, 10q23 and/or 10q24 or in the region of 10q23, 10q24 and/or 10q25 are provided. In particular embodiments of the markers associated with AD, the markers are located in a region of chromosome 10 about 30 cM around and including D10S583, or about 20 cM around and including D10S583, or about 15 cM around and including D10S583, or about 12 cM around and including D10S583, or about 10 cM around and including D10S583. In other particular embodiments of the markers associated with AD, the markers are located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. In further embodiments of the markers associated with AD, the markers are located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In yet further embodiments of the markers associated with AD, the markers are located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D110S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

Further particular regions of chromosome 10 in which markers associated with AD are located include: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive. A particular marker associated with AD provided herein is D10S583. Particular regions of chromosome 10 in which markers associated with AD are located can depend on several factors, including, for example, population.

In other embodiments of the markers provided herein, the markers are located in the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Genetic markers on chromosome 10 associated with AD yield scores or results indicative of association with AD or linkage disequilibrium with an AD DNA segment when tested in association or linkage disequilibrium assessment methods. Such methods include but are not limited to case-control studies and family-based association techniques. In addition, such methods can involve separate or combined tests for linkage of the marker to AD. Preferred methods are family-based association techniques. Particular markers associated with AD provided herein yield scores that may be considered significant evidence of association with AD or linkage disequilibrium with an AD DNA segment. For example, such a score would include a P value <0.5 and, in particular, a P value <0.5.

Particular markers associated with AD further include any genetic markers located on chromosome 10, including but not limited to the markers listed in FIG. 1, that yield scores or results indicative of association with AD or linkage disequilibrium with an AD DNA segment when tested in association assessment methods described herein and known to those of skill in the art. Included within these particular markers are any of the following markers that yield scores or results indicative of association with AD or linkage disequilibrium with an AD DNA segment when tested in association assessment methods: D10S564, D10S2470, D10S1755, D10S536, D10S185, D10S1171, D10S1173, D10S200, D10S1680, D10S520, D10S1736, D10S574, D10S571, D10S1690, ATA105C09, GATA30F07a, D10S677, D10S91, D10S1758, D10S577, D10S1709, D10S184, D10S1726, D10S198, D10S1123, D10S1147, D10S603, D10S1266, D10S1265, D10S1264, D10S1778, D10S192, FB7F11, D10S1267, D10S1692, D10S1738, D10S205, D10S1668, D10S1268, D10S222, D10S1697, GATA64B01, D10S540, D10S467, GATA114H09, D10S254, D10S1663, D10S530, D10S534, D10S521, D10S108, D10S1741, D10S1760, D10S1121, D10S1795, D10S1750, D10S1246, D10S597, D10S543, D10S88, D10S1682, D10S554, GATA83D05, D10S168, D10S1269, D10S1681, D10S1776, D10S562, D10S1748, D10S1773, D10S1731, ATA103C06, D10S468, D10S1237, D10S1158, D10S528, D10S1683, D10S544, D10S531, D10S1657, D10S545, D10S187, D10S1165, D10S1156, D10S221, D10S1425, D10S1693, D10S1722, D10S1141, D10S1236, D10S190, D10S503, GATA127H01, D10S1785, D10S542, D10S1701, D10S1792, D10S1740, D10S1757, D10S209, D10S1230, D10S1483, D10S1679, D10S587, D10S1708, D10S1723, D10S1213, D10S2322, D10S216, D10S1656, D10S12, D10S575, D10S214, D10S1703, D10S186, D10S1782, D10S1222, D10S1727, D10S217, D10S1676, D10S1134, D10S1655, GGAST14G01, D10S505, D10S1248, D10S1770, D10S1651, D10S590, D10S1675, 1QTEL23, D10S212, D10S555, D10S1711, D10S1700, D10S169, D10S1221, D10S1223, ATA25D02, D10S1229, D10S1232, ATA42E11, D10S1239, D10S1423, D10S1435, D10S110, D10S466, D10S525, D10S1148, D10S1149, D10S1150 and D10S1163.

Also provided herein are combinations of genetic markers on human chromosome 10 which are associated with AD. In particular embodiments, the markers in the combinations are located on chromosome 10q. Such combinations include markers located in the region of 10q22, 10q23, 10q24, 10q25 and/or 10q26. In further embodiments of the combinations of genetic markers, the markers are located in the region of 10q22, 10q23 and/or 10q24 or in the region of 10q23, 10q24 and/or 10q25. In particular embodiments, the markers contained in a combination of markers associated with AD are located in a region of chromosome 10 about 30 cM around and including D10S583, or about 20 cM around and including D10S583, or about 15 cM around and including D10S583, or about 12 cM around and including D10S583, or about 10 cM around and including D10S583. In other particular embodiments of the combinations of markers associated with AD, the markers are located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583. In further embodiments of the combinations of markers associated with AD, the markers are located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583. In yet further embodiments of the combinations of markers associated with AD, the markers are located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

In further embodiments of the combinations of genetic markers associated with AD, the markers are located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive. A particular marker associated with AD provided herein is D10S583. Particular regions of chromosome 10 in which markers that may be contained within the combinations of genetic markers are located can depend on several factors, including, for example, population.

In other embodiments of the combinations of genetic markers associated with AD provided herein, the markers are located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Combinations of genetic markers associated with AD have a variety of uses. For example, haplotypes associated with AD may be used in all the methods described herein that utilize one or more genetic markers associated with AD. Such haplotypes associated with AD thus may be used in methods of identifying the presence or absence in a subject of a combination of polymorphic markers associated with AD by analyzing chromosome 10, and in particular chromosome 10q, of the subject for such a combination, in methods of indicating a predisposition to or the occurrence of AD in a subject, methods of confirming a phenotypic diagnosis of AD in a subject, methods of predicting a response of a subject to a drug used to treat AD, methods of treating a subject manifesting an AD phenotype, and methods of identifying an AD DNA segment or gene as described herein.

5. Methods for Identifying Genetic Markers Associated with AD

Also provided herein are methods of identifying genetic markers associated with AD. The methods include a step of testing a polymorphic marker on chromosome 10 for association with AD. The testing may involve genotyping DNA from individuals affected with AD, and possibly also from related or unrelated individuals, with respect to the polymorphic marker and analyzing the genotyping data for association with AD using methods described herein and/or known to those of skill in the art. For example, statistical analysis of the data may involve a chi-squared or Fisher's exact test and may be conducted in conjunction with a number of programs, such as the transmission disequilibrium test (TDT), affected family based control test (AFBAC) and the haplotype relative risk test (HRR). Case-control strategies can be applied to the testing, as can, for example, TDT approaches.

In particular embodiments of the methods of identifying genetic markers associated with AD, the polymorphic marker is located in the region of 10q22, 10q23, 10q24, 10q25 or 10q26. In further embodiments, the marker is located on chromosome 10q22, 10q23 or 10q24. In yet further embodiments, the marker is located on chromosome 10q23, 10q24 or 10q25. Particular regions of chromosome 10 in which the polymorphic marker is located include: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

Further particular regions of chromosome 10 in which the polymorphic marker is located include: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

E. Linkage of AD on chromosome 10

Genetic markers linked to a DNA segment associated with AD have been identified and are described herein. The markers are located on human chromosome 10q. Particular markers are located on chromosome 10q22, 10q23, 10q24, 10q25 or 10q26. The markers tend to cosegregate with a DNA segment associated with AD, such as an AD gene, in families affected with AD. The markers can be identified through any linkage assessment methods described herein or known to those of skill in the art, and provide scores or results indicative of linkage to AD when tested by such linkage determination methods. The markers may be used in a variety of methods. For example, a method for detecting the presence or absence in a subject of a polymorphism linked to a DNA segment associated with Alzheimer's disease includes a step of analyzing chromosome 10 of the subject for a polymorphism linked to a DNA segment associated with Alzheimer's disease. A method for identifying a polymorphism linked to a DNA segment associated with Alzheimer's disease, includes a step of analyzing a genetic marker on chromosome 10 for linkage to Alzheimer's disease characterized by a significant or highly significant LOD score. Another method for identifying a polymorphism linked to a DNA segment associated with Alzheimer's disease, includes a step of analyzing a genetic marker on chromosome 10 for linkage to Alzheimer's disease, wherein the marker is located in the region of 10q22, 10q23, 10q24, 10q25 or 10q26.

1. Genetic Linkage

The closer together two sequences are on a chromosome, the less likely that a recombination event will occur between them, and the more closely linked they are. Thus, the recombination frequency, i.e., the probability that there is a recombination event between two loci (also referred to as the recombination fraction), can be used as a measure of the genetic distance between two gene loci. A recombination frequency of 1% is equivalent to 1 map unit, or 1 centimorgan (cM), which is roughly equivalent to 1,000 kb of DNA. Loci that segregate independently within a family are unlinked and have a recombination fraction of 50%, whereas linked loci cosegregate within a family and have a recombination fraction of less than about 50%. For example, genetic markers linked to a DNA segment associated with AD on chromosome 10 may have a recombination fraction of less than about 50%, or about 45% or less, or about 40% or less, or about 35% or less, or about 30% or less, or about 25% or less, or about 20% or less, or about 15% or less, or about 10% or less, or about 5% or less or about 2.5% or less, or about 2% or less, or about 1.5% or less or about 1% or less or about 0.5% or less, or about 0.1% or less, or about 0. The particular recombination fraction depends on the particular marker.

For example, in terms of the genetic distance between the linked markers on chromosome 10 and a DNA segment associated with AD on chromosome 10, the markers may be less than about 85 cM from the DNA segment, or less than about 80 cM from the DNA segment, or less than about 75 cM from the DNA segment, or less than about 70 cM from the DNA segment, or less than about 65 cM from the DNA segment, or less than about 60 cM from the DNA segment, or less than about 55 cM from the DNA segment, or less than about 50 cM from the DNA segment, or less than about 45 cM from the DNA segment, or less than about 40 cM from the DNA segment, or less than about 35 cM from the DNA segment, or less than about 30 cM from the DNA segment, or less than about 25 cM from the DNA segment, or less than about 20 cM from the DNA segment, or less than about 15 cM from the DNA segment, or less than about 10 cM from the DNA segment, or less than about 5 cM from the DNA segment, or less than about 4 cM from the DNA segment, or less than about 3 cM from the DNA segment, or less than about 2 cM from the DNA segment, or less than about 1.5 cM from the DNA segment, or less than about 1.0 cM from the DNA segment, or less than about 0.75 cM from the DNA segment, or less than about 0.5 cM from the DNA segment or less than about 0.25 cM from the DNA segment, or less than about 0.2 cM from the DNA segment or less than about 0.15 cM from the DNA segment or less than about 0.1 cM from the DNA segment. The particular distance depends on the particular marker. The linked markers on chromosome 10 may be located within a DNA segment associated with AD and may be a polymorphism in an AD gene, such as, for example, a polymorphism in an AD gene that is responsible for a defect in an AD gene. When the marker is located within an AD gene, it is referred to as coincident with the gene.

If two loci are situated on different chromosomes, the transmission of alleles from generation to generation of each locus will be random and they are said to be “unlinked.” If two loci are situated on the same chromosome, the transmission of alleles of one locus will be affected by the presence of the other locus such that the ratios of alleles are no longer independent, and the loci are referred to as “linked.” Thus, two loci may be said to be linked when they are located relatively close together on the same chromosome. Genetic markers provided herein are located sufficiently close to a DNA segment associated with AD on chromosome 10 such that the marker and DNA segment are linked.

For example, in terms of the physical distance between the linked markers and a DNA segment associated with AD on chromosome 10, the markers may be less than about 72 Mb from the DNA segment, or less than about 65 Mb from the DNA segment, or less than about 63 Mb from the DNA segment, or less than about 59 Mb from the DNA segment, or less than about 55 Mb from the DNA segment, or less than about 50 Mb from the DNA segment, or less than about 45 Mb from the DNA segment, or less than about 40 Mb from the DNA segment, or less than about 35 Mb from the DNA segment, or less than about 30 Mb from the DNA segment, or less than about 25 Mb from the DNA segment, or less than about 20 Mb from the DNA segment, or less than about 15 Mb from the DNA segment, or less than about 10 Mb from the DNA segment, or less than about 5 Mb from the DNA segment, or less than about 2.5 Mb from the DNA segment, or less than about 1 Mb from the DNA segment, or less than about 0.5 Mb from the DNA segment, or less than about 0.1 Mb from the DNA segment, or less than about 0.05 Mb from the DNA segment, or less than about 0.01 Mb from the DNA segment, or less than about 0.005 Mb from the DNA segment, or less than about 0.001 Mb from the DNA segment. The particular distance depends on the particular marker.

Two loci are completely linked when there is no recombination between them; the same alleles or phenotypes are always transmitted together from generation to generation within a family. An intermediate state of linkage, referred to as “incomplete linkage” occurs when the transmission of alleles of two loci deviates consistently and measurably from independent assortment (e.g., random transmission of alleles located on different chromosomes) but a consistent recombination fraction nonetheless exists for the loci [see, e.g., March (1999) Mol. Biotechnol. 13:113-122].

2. Analysis of Genetic Linkage

Linkage analysis is based upon establishing a correlation between the transmission of genetic markers and that of a specific trait or trait gene throughout generations within a family. Thus the aim of linkage analysis is to detect marker loci that show cosegregation with a trait of interest in a pedigree.

a. Procedures

In conducting linkage analysis, two positions on a chromosome are followed from one generation to the next within a family to determine the frequency of recombination between them. This can be accomplished by genotyping DNA from fully informative individuals within pedigrees and counting recombinants and nonrecombinants. In a study of an inherited disease, such as AD, one chromosomal position, or locus, is marked by the disease gene and the other position is marked by a DNA sequence (referred to as genetic marker) that shows natural variation in the population, e.g., variable number of tandem repeats (VNTRs), such as minisatellites and microsatellites, single nucleotide polymorphisms (SNPs) and restriction fragment length polymorphisms (RFLPs). RFLPs are variations that modify the length of a restriction fragment. Minisatellites are tandemly repeated DNA sequences present in units of about 5-50 or more repeats which are distributed along regions of human chromosomes ranging from 0.1-20 kb in length. Microsatellites are tandemly repeated DNA sequences typically present in repeats of lesser units, e.g., up to 4 repeats, than those of minisatellites. Because microsatellites and minisatellites present many possible alleles, their informative content is very high. SNPs are densely spaced in the human genome and represent the most frequent type of variation.

Inheritance of a marker can be determined by analyzing DNA from each individual for the presence or absence of the marker whereas inheritance of the disease gene can be determined by examining whether the individual displays symptoms of the disease or is a parent of an affected individual or not. In every family, the inheritance of the genetic marker is compared to the inheritance of the disease state.

Linkage analysis may be two-point, i.e., comparing the segregation of a marker and a disease, or multipoint, i.e., simultaneous analysis of linkage between the disease and several genetic markers. Multipoint analysis can be advantageous in mapping a disease gene. For example, the informativeness of the pedigree is usually increased in multipoint analysis. Each pedigree has a certain amount of potential information, dependent on the number of parents heterozygous for the marker loci and the number of affected individuals in the family. However, not all markers are sufficiently polymorphic as to be informative in all those individuals. If multiple markers are considered simultaneously, then the probability of an individual being heterozygous for at least one of the markers is greatly increased. In addition, an indication of the position of the disease gene among the markers may be determined in multipoint analysis. This may allow identification of flanking markers, and thus eventually allows isolation of a small region in which the disease gene resides. Examples of computer software which may be used for multipoint analysis include GENEHUNTER-PLUS [Kruglyak et al. (1996) Am. J. Hum. Genet. 58:1347; Kong and Cox (1997) Am. J. Hum. Genet. 61:1179], ASPEX [see, e.g., Badner et al. (1998) Am. J. Hum. Genet. 63:880-888; Hauser et al. (1996) Genet. Epidemiol 13:117-137; Davis and Weeks (1997) Am. J. Hum. Genet. 61:1431-1444] and LINKAGE [see Lathrop et al. (1984) Proc. Natl. Acad. Sci. U.S.A. 81:3443-3446].

b. Linkage Measurement

Linkage may be assessed by the LOD (logarithm of an odds ratio) score method [Morton (1955) Am. J. Hum. Genet. 7:277-318; Rice et al. (2001) Adv. Genet. 42:99-113] or other acceptable statistical linkage determination [see also Ott (1991) Analysis of Human Genetic Linkage, Baltimore, London, John Hopkins; Terwilliger and Ott (1994) Handbook of Human Genetic Linkage, Baltimore, John Hopkins University Press; Strachan and Read (1996) Human Molecular Genetics, Oxford. BIOS Scientific Publishers Ltd.; Sudbery (1998) Human Molecular Genetics, Harlow, Addison Wesley Longman; Lander and Schork (1994) Science 265:2037-2048]. In linkage analysis, a series of likelihood ratios (relative odds) at various possible values of Θ, ranging from 0 (no recombination) to 0.50 (random assortment) are calculated. The computed likelihoods are usually expressed as the logarithm of the likelihood ratio (LOD). The use of logarithms allows data collected from different families to be combined by simple addition. Computer programs are available that run the analyses involved in statistical linkage determination [see, e.g., LIPED; MLINK; Lathrop et al. (1984) Proc. Natl. Acad. Sci. U.S.A. 81:3443-3446; Terwilliger and Ott (1994) Handbook of Human Genetic Linkage, Baltimore, John Hopkins University Press and http://linkage.rockefeller.edu/soft/list.html]. A LOD score is the logarithm of the ratio of the likelihood that two loci are linked at a given distance (or recombination fraction Θ) to the likelihood that they are not linked (recombination fraction Θ=0.5; greater than 50 cM apart). The value of Θ at which the LOD score is the highest is considered to be the best estimate of the recombination fraction, the “maximum likelihood estimate.”

Positive LOD scores can be considered as evidence of linkage. Genetic markers on chromosome 10 linked to a DNA segment associated with AD yield positive LOD scores when analyzed for linkage to AD by a LOD score method. The positive LOD score may be greater than or equal to about 1.0, or greater than or equal to about 1.5, or greater than or equal to about 1.9, or greater than or equal to 2.0, or greater than or equal to about 2.2, or greater than or equal to about 2.6, or greater than or equal to about 2.7, or greater than or equal to about 2.8, or greater than or equal to about 3.0, or greater than or equal to about 3.12, or greater than or equal to about 3.2, or greater than or equal to about 3.5, or greater than or equal to about 4.0, or greater than or equal to about 4.5, or greater than or equal to about 5.0, or greater than or equal to about 5.4, or greater than or equal to about 5.5, or greater than or equal to about 6.0, or greater than or equal to about 6.5, or greater than or equal to about 7.0, or greater than or equal to about 7.5, or greater than or equal to about 8.0, or greater than or equal to about 8.5, or greater than or equal to about 9.0, or greater than or equal to about 9.5, or greater than or equal to about 10.0 or greater than or equal to about 10.5, or greater than or equal to about 11.0, or greater than or equal to about 11.5, or greater than or equal to about 12.0, or greater than or equal to about 12.5, or greater than or equal to about 13.0 or greater than or equal to about 13.5, or greater than or equal to about 14.0 or greater than or equal to about 14.5, or greater than or equal to about 15.0, or greater than or equal to about 15.5, or greater than or equal to about 16.0, or greater than or equal to about 16.5, or greater than or equal to about 17.0. The particular LOD score depends on the particular marker.

Criteria have been proposed for use in categorizing linkage analysis results in terms of the extent to which the results may serve as evidence of linkage between loci. For example, by some criteria [Morton (1955) Am. J. Hum. Genet. 7:277-318], a LOD score of 1.5 or greater is considered to be “suggestive” of linkage whereas a LOD score of 3 is considered as statistically significant evidence for linkage. The significance level, α, is that which is associated with a likelihood ratio test computed to the base e−X²=lod(2ln10). For a LOD score of 3, α≈0.0001; for a LOD score of 1.5, α<0.004. It has also been proposed that a multipoint LOD score of ≦5.4 may be considered as “highly significant” evidence of linkage, whereas “significant” evidence of linkage may be viewed as a multipoint LOD score ≦3.6 or a two-point LOD score ≦3.3, and “suggestive” evidence of linkage is provided by multipoint LOD scores ≦2.2 or two-point LOD scores ≦1.9 [see, e.g., Lander and Kruglyak (1995) Nat. Genet. 11:241].

Linkage analysis methods can be used to screen the entire human genome for one or more chromosomal regions containing loci linked to a disease gene. In genome screening procedures, DNA from individual members of families in which one or more family members are trait positive is typed with respect to a set of genetic markers that includes multiple markers from each human chromosome. The resolution of the screen depends on the number of markers that are typed and the distance between the markers on each chromosome. Generally, the higher the density of the collection of markers, the higher the resolution of the mapping results. Typically, markers separated by an average distance of 10 cM or less are considered to provide for a fairly high resolution genome screen. In particular, an average marker separation of 9 cM or less is used for high-resolution mapping. The results of the typing are compared to the disease status of each individual, and these data are statistically evaluated using one or more of a variety of linkage analysis computer software programs [see, e.g., O'Connell and Weeks (1995) Nat. Genet. 11:402-408 describing the VITESSE algorithm]. Traditional LOD score analysis is a strong method for evaluating linkage in forms of a disorder showing obvious Mendelian inheritance, but weakens when the mode of transmission is complex and genetic parameters cannot be accurately specified. In such cases, statistical evaluation of genotyping data may be strengthened through use of allele sharing linkage methods in pairs of affected siblings or other relative pairs and association studies. Such methods are described herein.

C. Statistical Methods in Linkage Analysis

Methods of analyzing genetic linkage are termed parametric (i.e., “model-based”) if gene frequency and penetrance must be estimated, and nonparametric otherwise. There are many models within each class.

(1) Parametric Linkage Analysis

Parametric linkage analysis [see, e.g., Ott (1991) Analysis of Human Genetic Linkage, Baltimore, London, John Hopkins and Terwilliger and Ott (1994) Handbook of Human Genetic Linkage, Baltimore, John Hopkins University Press] applied to large pedigrees with many affected individuals can be useful in the identification of highly penetrant genes. A number of computer software programs are available to conduct parametric linkage analysis [see, e.g., FASTLINK; Lathrop et al. (1984) Proc. Natl. Acad. Sci. U.S.A. 81:3443-3446; Cottingham et al. (1993) Am. J. Hum. Genet. 53:252-263; Schaffer et al. (1994) Hum. Heredity 44:225-237].

Parametric linkage analysis can be limited due to its reliance on the choice of a genetic model suitable for a particular trait, and may be difficult when applied to the analysis of complex genetic traits such as those due to the combined action of multiple genes and/or environmental factors. In the mapping of diseases lacking a clear Mendelian inheritance pattern or caused by several genes of low to moderate penetrance, it may be more suitable to utilize nonparametric analysis applied to small sets of affected relatives, such as affected sib pairs.

(2) Nonparametric Linkage Analysis

Nonparametric linkage analysis involves determining whether the inheritance pattern of a chromosomal region is not consistent with random Mendelian segregation by showing that affected relatives inherit identical copies of the region (i.e., allele sharing) more often than expected by chance. Distortions from expected ratios of allele sharing among relatives [usually sibs; see, e.g., Risch (1990) Am. J. Hum. Genet. 46:229-241] who share a disease phenotype are tested. This form of analysis is independent of the mode of inheritance of the disease and thus is well-suited for cases in which there is not an absolute correlation between phenotype and genotype, such as in many multifactorial traits in which multiple genes may contribute to observed phenotype.

Typically, nonparametric linkage analysis is based in the analysis of the proportion of alleles shared identical by descent (IBD) between two sibs affected with a disease (affected sib pairs). The degree of agreement at a marker locus in two individuals can also be measured by the number of alleles identical by state (IBS). Nonparametric linkage analysis can be used in genome wide scans of multifactorial diseases using linkage maps of genetic markers, e.g., microsatellite markers. A number of computer software programs are available to conduct nonparametric linkage analysis [see, e.g., MAPMAKER/SIBS, Lander and Kruglyak (1995) Am. J. Hum. Genet. 57:439-454; GENEHUNTER-PLUS, Kruglyak et al. (1996) Am. J. Hum. Genet. 58:1347; SIMIBD, Davis et al. (1996) Am. J. Hum. Genet. 58:867-880; ASPEX (MLS), Risch (1990) Am. J. Hum. Genet. 46:222-253].

3. Analysis of Genetic Linkage of AD on Chromosome 10

Genotyping data with respect to multiple markers on human chromosome 10 in AD families were analyzed using several different analytic strategies and models (see EXAMPLE 2, i.e., using both autosomal dominant and recessive parametric two-point analyses and nonparametric two-point and multipoint analyses) and using a Family-Based Association Test (FBAT) for association with AD (see EXAMPLE 3). As described in detail herein, LOD scores indicative of linkage of AD obtained for markers on chromosome 10 were consistently achieved in each of these analytical tests.

Chromosome 10 markers D10S583, D10S1710, D10S566, D10S1671 and D10S1741 each yielded LOD scores >1 for the whole sample in at least one of the AD linkage analyses that were conducted. Analysis of markers D10S583 and D10S1671 revealed significant evidence of linkage to AD. The greatest linkage scores obtained in each of the analyses of the whole sample were as follows: parametric two-point LOD score (Zₘₐₓ) using the dominant model=3.3 for D10S583; parametric two-point LOD score (Zₘₐₓ) using the recessive model=2.9 for D10S1671; nonparametric two-point linkage score (Zₗᵣ)=3.3 for D10S1671; nonparametric multipoint LOD score=1.9 for D10S1710. The greatest linkage scores obtained in each of the analyses of the late-onset sample were as follows: parametric two-point LOD score (Zₘₐₓ) using the dominant model=3.4 for D10S1671; parametric two-point LOD score (Zₘₐₓ) using the recessive model=3.8 for D10S1671; nonparametric two-point linkage score (Zₗᵣ)=3.8 for D10S1671; nonparametric multipoint LOD score=2.1 for D10S1710.

Although for each of the specified analyses, one of markers D10S583, D10S1671 and D10S1710 yielded the greatest linkage score, the second and sometimes third highest linkage scores in each analysis, were, for the most part, also indicative of linkage to AD. Thus, the region of chromosome 10q around and between these markers is referred to as a region of peak linkage which may include one or more specific peaks. Markers flanking this region of peak linkage include the terminus of the q arm of chromosome 10 and D10S564.

Stratification of the sample into APOE ε4/4-positive and -negative subsets resulted in a lowering of linkage scores obtained for each marker when analyzed in parametric two-point linkage analyses using a dominant disease model. The linkage scores generally were, however, more pronounced in families without the APOE ε414 genotype.

Linkage-based genetic analysis tends to localize disease-susceptiblity genes that have a more substantial effect size. The effect size of a disease gene is the degree to which mutations or polymorphisms in a gene confer susceptibility to the disease taking into account the magnitude of prevalence and penetrance of the polymorphism. The prevalence of a polymorphic allele refers to the percentage of affected individuals who carry the polymorphism. Penetrance of a polymorphic allele refers to the percent chance that a carrier of the allele will express the phenotype. The concept of penetrance establishes the connection between genotypes and phenotypes and thus is of central importance in linkage analysis.

The relative effect size of a gene may be estimated based on the magnitude of LOD scores obtained in linkage analyses relating to the gene. Maximum LOD scores obtained in analyses of linkage of AD on chromosome 10 described herein are similar to the maximum LOD scores reported in linkage analyses that lead to the discovery of APOE as an AD-associated gene [Pericak-Vance et al. (1991) Am. J. Human Genet. 48:1034-1050]. The significant linkage of AD on chromosome 10 indicates that the one or more AD DNA segments or AD genes located on chromosome 10 have a considerable effect size which is comparable to or greater than that of APOE.

Alleles can be considered based on phenotypic classes [see, e.g., Lio and Morton (1997) Proc. Natl. Acad. Sci. U.S.A. 94:5344-5348]. One class of alleles, referred to as major genes, can be characterized by segregation analysis. These genes tend to be rare and have megaphenic effects (which can be measured as displacement between homozygotes) that are large relative to the standard deviation of liability. A major gene is sufficient to cause affection against almost any genetic background, and therefore interaction is negligible except for modifiers of expression. Another class of alleles, polygenes, tend to be common and have microphenic effects too small to be characterized. These alleles may possibly be detected, however, through allelic association at candidate loci. Between these two extreme classes of alleles are oligogenes, also called leading factors [Wright (1968) Evolution and the Genetics of Populations 1:411-417]. These alleles are common and have mesophenic effects too small to be reliably characterized by segregation analysis, but in large samples they can be detected by nonparametric methods and elucidated by combined segregation and linkage analysis, including allelic association as coupling frequencies [MacLean et al. (1984) Comput. Biomed. Res. 17:471-480; Shields et al. (1994) Am. J. Hum. Genet. 55:540-554]. Small numbers of oligogenes interact to produce affection. One locus may have all three allelic classes; thus, small effects may be detected through allelic association at loci recognized as candidates through larger effects.

4. Genetic Markers Linked to an AD-Associated DNA Segment on Chromosome 10

The results of linkage analyses of genotyping data with respect to markers located on chromosome 10 in AD families revealed genetic markers on chromosome 10 that are linked to one or more DNA segments, e.g., one or more genes, associated with AD. Included within the linked markers are markers located in the region of 10q22, 10q23, 10q24, 10q25 or 10q26. Particular regions of chromosome 10 in which markers linked to one or more DNA segments associated with AD are located include: (1) the region extending about 45 cM, or about 42 cM, or about 33 cM, or about 30 cM, or about 25 cM, or about 20 cM, or about 15 cM, or about 10 cM, or about 5 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, D10S1710 and/or D10S1671, (2) the region extending about 62 cM, or about 59 cM, or about 50 cM, or about 47 cM, or about 32 cM, or about 30 cM, or about 28 cM, or about 25 cM, or about 20 cM, or about 17 cM, or about 15 cM, or about 10 cM, or about 5 cM, or about 2.5 cM, or about 1 cM, or about 0.5 cM or about 0.1 cM distal (telomeric) from and including marker D10S564, D10S583, D10S1710 and/or D10S1671, (3) the region extending from and including any of markers D10S564, D10S583, D10S1710, D10S1671 or D10S1741 to the terminus of the q arm (4) the region extending about 46 Mb, or about 40 Mb, or about 32 Mb, or about 25 Mb, or about 20 Mb, or about 16 Mb, or about 10 Mb, or about 5 Mb, or about 2 Mb, or about 1 Mb, or about 0.5 Mb, or about 0.3 Mb, or about 0.19 Mb, or about 0.1 Mb, or about 0.05 Mb, or about 0.01 Mb, or about 1 kb proximal from and including marker D10S583, D10S1710 and/or D10S1671, (5) the region extending about 50 Mb, or about 48 Mb, or about 39 Mb, or about 35 Mb, or about 30 Mb, or about 25 Mb, or about 20 Mb, or about 15 Mb, or about 10 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S564, D10S583, D10S1710 and/or D10S1671, (6) the region between D10S564 and D10S583, inclusive, (7) the region between D10S564 and D10S1710, inclusive, (8) the region between D10S564 and D10S566, inclusive, (9) the region between D10S564 and D10S1671, inclusive, (10) the region between D10S564 and D10S1741, inclusive, (11) the region between the terminus of the q arm of chromosome 10 and D10S1741, inclusive, (12) the region between markers D10S1741 and D10S1671, inclusive, (13) the region between markers D10S1741 and D10S566, inclusive, (14) the region between markers D10S1741 and D10S1710, inclusive, (15) the region between markers D10S1741 and D10S583, inclusive and (16) the region between markers D10S1741 and D10S564, inclusive. Particular markers linked to a DNA segment associated with AD include D10S583, D10S1710 and D10S1671.

Genetic markers on chromosome 10 linked to a DNA segment associated with AD yield scores or results indicative of linkage to AD when tested in linkage assessment methods. Such methods include but are not limited to LOD score methods, including LOD score methods involving parametric or nonparametric linkage analysis and/or two-point or multipoint analyses. Particular chromosome 10 markers linked to a DNA segment associated with AD yield LOD scores that may be considered highly significant, significant, suggestive or indicative of evidence of linkage.

Particular markers linked to a DNA segment associated with AD further include any genetic markers located on chromosome 10, including but not limited to the markers listed in FIG. 1, that yield scores or results indicative of linkage to AD when tested in linkage assessment methods described herein and known to those of skill in the art. Included within these particular markers are any of the following markers that yield scores or results indicative of linkage to AD when tested in linkage assessment methods: D10S564, D10S2470, D10S1755, D10S536, D10S185, D10S1171, D10S1173, D10S200, D10S1680, D10S520, D10S1736, D10S574, D10S571, D10S1690, ATA105C09, GATA30F07a, D10S677, D10S91, D10S1758, D10S577, D10S1709, D10S184, D10S1726, D10S198, D10S1123, D10S1147, D10S603, D10S1266, D10S1265, D10S1264, D10S1778, D10S192, FB7F11, D10S1267, D10S1692, D10S1738, D10S205, D10S1668, D10S1268, D10S222, D10S1697, GATA64B01, D10S540, D10S467, GATA114H09, D10S254, D10S1663, D10S530, D10S534, D10S521, D10S108, D10S1741, D10S1760, D10S1121, D10S1795, D10S1750, D10S1246, D10S597, D10S543, D10S88, D10S1682, D10S554, GATA83D05, D10S168, D10S1269, D10S1681, D10S1776, D10S562, D10S1748, D10S1773, D10S1731, ATA103C06, D10S468, D10S1237, D10S1158, D10S528, D10S1683, D10S544, D10S531, D10S1657, D10S545, D10S187, D10S1165, D10S1156, D10S221, D10S1425, D10S1693, D10S1722, D10S1141, D10S1236, D10S190, D10S503, GATA127H01, D10S1785, D10S542, D10S1701, D10S1792, D10S1740, D10S1757, D10S209, D10S1230, D10S1483, D10S1679, D10S587, D10S1708, D10S1723, D10S1213, D10S2322, D10S216, D10S1656, D10S12, D10S575, D10S214, D10S1703, D10S186, D10S1782, D10S1222, D10S1727, D10S217, D10S1676, D10S1134, D10S1655, GGAST14G01, D10S505, D10S1248, D10S1770, D10S1651, D10S590, D10S1675, 1QTEL23, D10S212, D10S555, D10S1711, D10S1700, D10S169, D10S1221, D10S1223, ATA25D02, D10S1229, D10S1232, ATA42E11, D10S1239, D10S1423, D10S1435, D10S110, D10S466, D10S525, D10S1148, D10S1149, D10S1150 and D10S1163.

5. Methods for Identifying Genetic Markers Linked to an AD-Associated DNA Segment

Methods of identifying genetic markers linked to a DNA segment associated with AD can include a step of testing a polymorphic marker on chromosome 10 for linkage to AD. The testing may involve genotyping DNA from individuals in AD families with respect to the polymorphic marker and analyzing the genotyping data for linkage to AD using methods described herein and/or known to those of skill in the art. For example, the data may be analyzed in a LOD score determination method.

In particular methods of identifying genetic markers linked to a DNA segment associated with AD, the polymorphic marker is located in the region of 10q22, 10q23, 10q24, 10q25 or 10q26. Particular regions of chromosome 10 in which the polymorphic marker is located include: (1) the region extending about 45 cM, or about 42 cM, or about 33 cM, or about 30 cM, or about 25 cM, or about 20 cM, or about 15 cM, or about 10 cM, or about 5 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, D10S1710 and/or D10S1671, (2) the region extending about 62 cM, or about 59 cM, or about 50 cM, or about 47 cM, or about 32 cM, or about 30 cM, or about 28 cM, or about 25 cM, or about 20 cM, or about 17 cM, or about 15 cM, or about 10 cM, or about 5 cM, or about 2.5 cM, or about 1 cM, or about 0.5 cM or about 0.1 cM distal (telomeric) from and including marker D10S564, D10S583, D10S1710 and/or D10S1671, (3) the region extending from and including any of markers D10S564, D10S583, D10S1710, D10S1671 or D10S1741 to the terminus of the q arm (4) the region extending about 46 Mb, or about 40 Mb, or about 32 Mb, or about 25 Mb, or about 20 Mb, or about 16 Mb, or about 10 Mb, or about 5 Mb, or about 2 Mb, or about 1 Mb, or about 0.5 Mb, or about 0.3 Mb, or about 0.19 Mb, or about 0.1 Mb, or about 0.05 Mb, or about 0.01 Mb, or about 1 kb proximal from and including marker D10S583, D10S1710 and/or D10S1671, (5) the region extending about 50 Mb, or about 48 Mb, or about 39 Mb, or about 35 Mb, or about 30 Mb, or about 25 Mb, or about 20 Mb, or about 15 Mb, or about 10 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S564, D10S583, D10S1710 and/or D10S1671, (6) the region between D10S564 and D10S583, inclusive, (7) the region between D10S564 and D10S1710, inclusive, (8) the region between D10S564 and D10S566, inclusive, (9) the region between D10S564 and D10S1671, inclusive, (10) the region between D10S564 and D10S1741, inclusive, (11) the region between the terminus of the q arm of chromosome 10 and D10S1741, inclusive, (12) the region between markers D10S1741 and D10S1671, inclusive, (13) the region between markers D10S1741 and D10S566, inclusive, (14) the region between markers D10S1741 and D10S1710, inclusive, (15) the region between markers D10S1741 and D10S583, inclusive and (16) the region between markers D10S1741 and D10S564, inclusive. Particular markers linked to a DNA segment associated with AD include D10S583, D10S1710 and D10S1671.

F. Location of One or More DNA Segments Associated with AD on Chromosome 10

The discovery of genetic association with AD on human chromosome 10 as described herein identifies chromosome 10 as the location of one or more DNA segments associated with AD. The type of AD may be late-onset AD. The DNA segment or segments may be genes. The DNA segment or segments may be an AD DNA segment or AD gene and may be causative of AD. In particular, chromosome 10 is identified as containing at least one DNA segment or gene associated with AD and of comparable or greater impact than APOE, wherein the effect size of the gene is comparable to or greater than that of APOE.

In one embodiment of a DNA segment associated with AD on chromosome 10, the DNA segment is an allele on chromosome 10 that confers in those who carry the allele protection against AD relative to those who do not carry the allele. In another embodiment of a DNA segment associated with AD on chromosome 10, the DNA segment either directly causes or confers an increased susceptibility to AD (e.g., a “risk” or “disease” allele). In particular embodiments, the DNA segment is an allele on chromosome 10q that confers in those who carry the allele protection against AD relative to those who do not carry the allele, or either directly causes or confers an increased susceptibility to AD (e.g., a “risk” or “disease” allele).

At least one, or two or more, DNA segment(s) associated with AD may be located in the regions of 10q22, 10q23, 10q24, 10q25 or 10q26. In particular, at least one, or two or more, DNA segment(s) associated with AD may be located in the regions of 10q22, 10q23 or 10q24. At least one, or two or more, DNA segment(s) associated with AD may be located in the regions of 10q23, 10q24 or 10q25. Particular regions of chromosome 10 in which a DNA segment associated with AD may be located include: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

Further particular regions of chromosome 10 in which a DNA segment associated with AD may be located include: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

G. Methods for Identifying an AD DNA Segment or AD Gene

Methods for identifying an AD DNA segment or an AD gene are provided herein. The methods are based in the genetic analysis of chromosome 10 of individuals affected with AD. In particular embodiments, the methods are based in the genetic analysis of chromosome 10q. In further embodiments, the methods may be based in genetic analysis of chromosome 10q22, 10q23, 10q24, 10q25 or 10q26. In particular embodiments, the methods may be based in genetic analysis of chromosome 10 within a region identified herein as containing or near markers associated with AD. In yet further embodiments, the methods may be based in genetic analysis of chromosome 10q22, 10q23 or 10q24. In particular embodiments, the methods may be based in genetic analysis of chromosome 10q23, 10q24 or 10q25.

Genetic analysis of chromosome 10 may involve methods which include, but are not limited to, any one or more of the following: analysis of one or more genetic markers on chromosome 10 for linkage to AD or a DNA segment associated with AD; analysis of association between one or more genetic markers on chromosome 10 and AD; analysis of association between a chromosome 10 haplotype and AD; calculation of linkage disequilibrium values from chromosome 10 haplotype data; sequencing of DNA in chromosome 10 for polymorphic markers, in particular, SNPs, as genetic markers in further genetic analyses as described above or as possible AD DNA segments or AD gene variants underlying AD.

1. Linkage Analysis in the Identification of AD DNA Segments or Genes

The search for disease-susceptibility genes generally may be conducted using two main analytical methods: linkage analysis, in which evidence is sought for cosegregation between a locus and a putative trait locus within families, and association analysis, in which evidence is sought for a statistically significant association between an allele and a trait or a trait-causing allele [Khoury et al. (1993) Fundamentals of Genetic Epidemiology, Oxford University Press, N.Y.]. These methods can be viewed as tools which may be applied in any of several approaches to disease gene discovery. Two primary approaches to disease gene discovery are genetic localization and candidate gene studies.

a. Candidate Gene Approach

The candidate gene approach typically takes into account knowledge of biological processes of a disease as a basis for selecting genes that encode proteins that could be envisioned to be involved in the biological processes. For example, reasonable candidate genes for blood pressure disorders could be proteins and enzymes involved in the renin-angiotensin system. Candidate genes can be evaluated genetically as possible disease genes by linkage and/or association studies of markers in the candidate gene region.

(1) Methods of Identifying a Candidate AD Gene

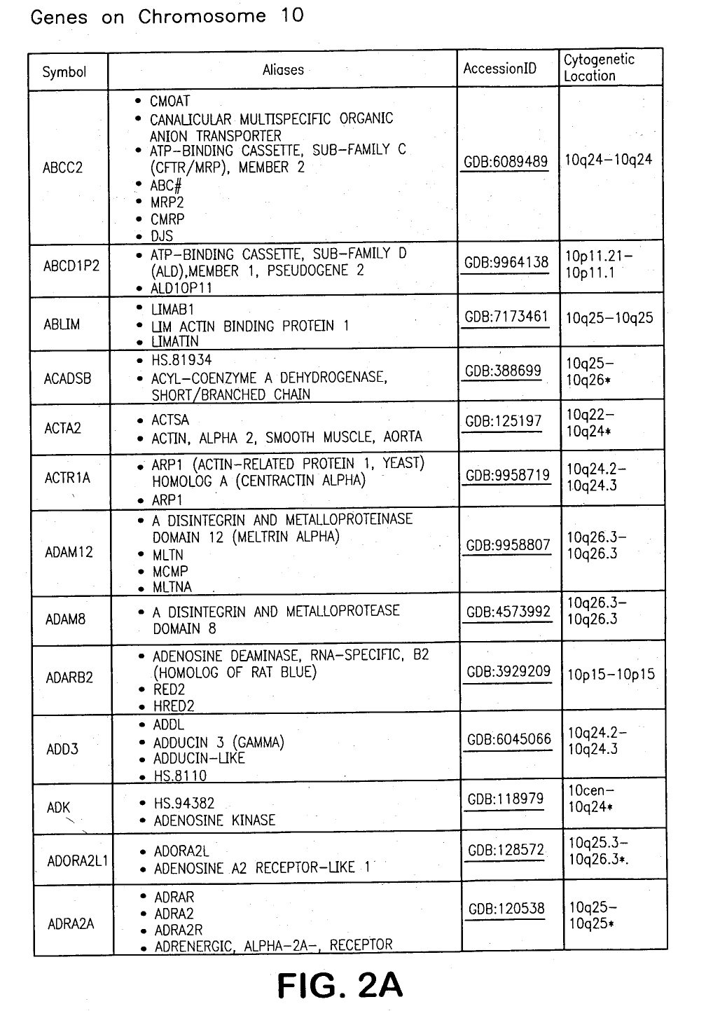

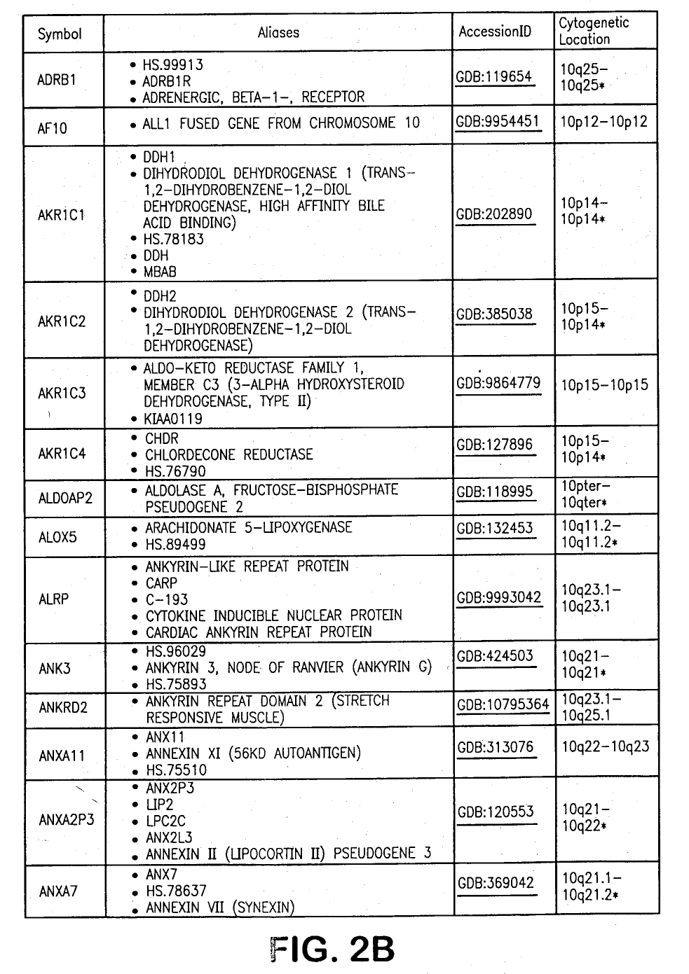

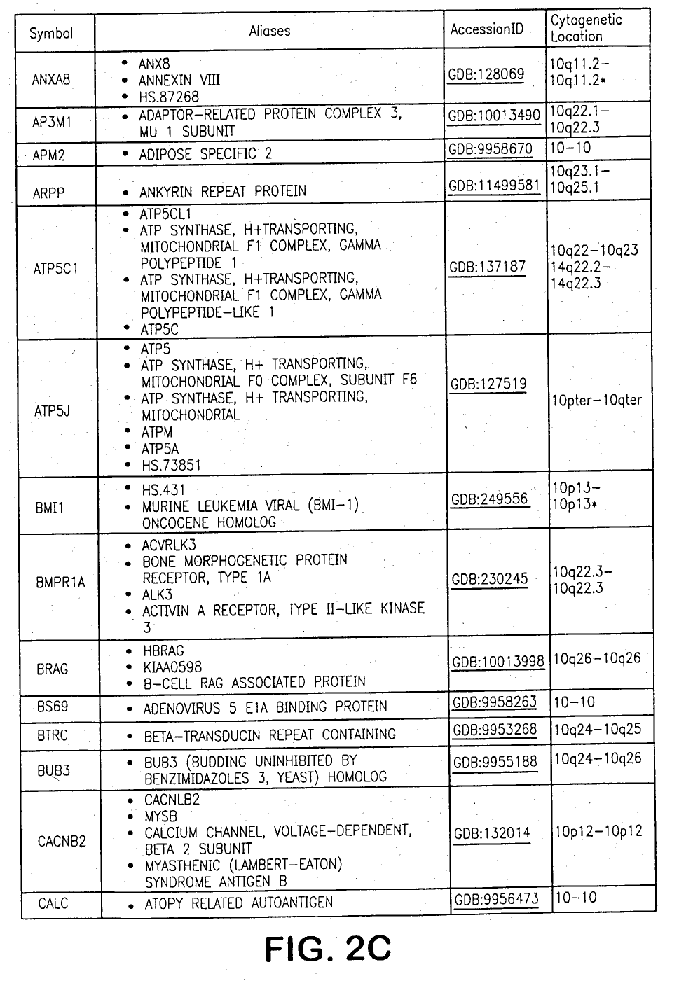

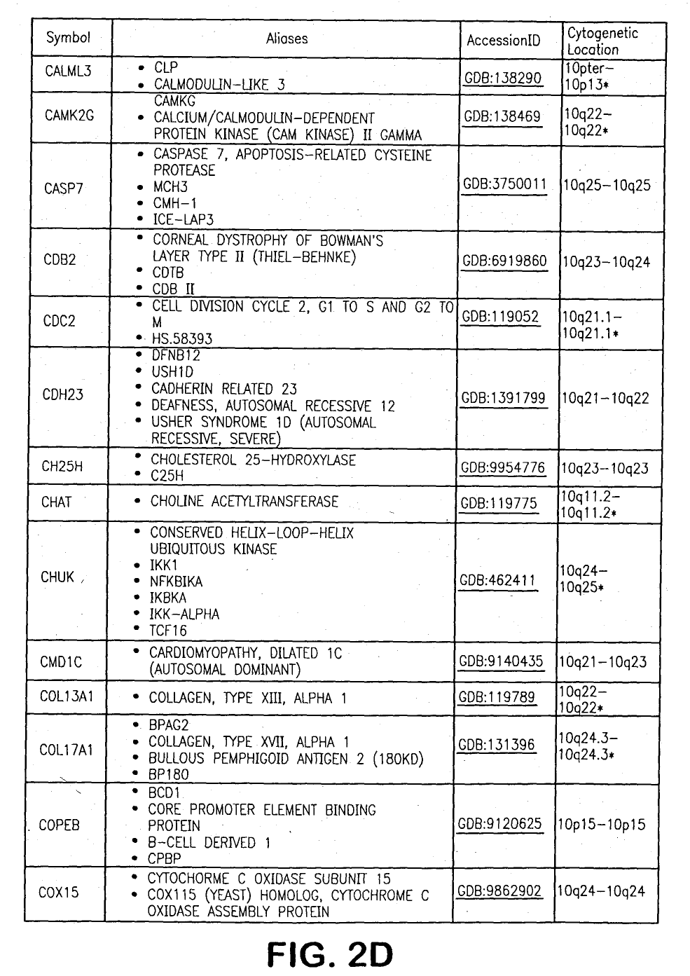

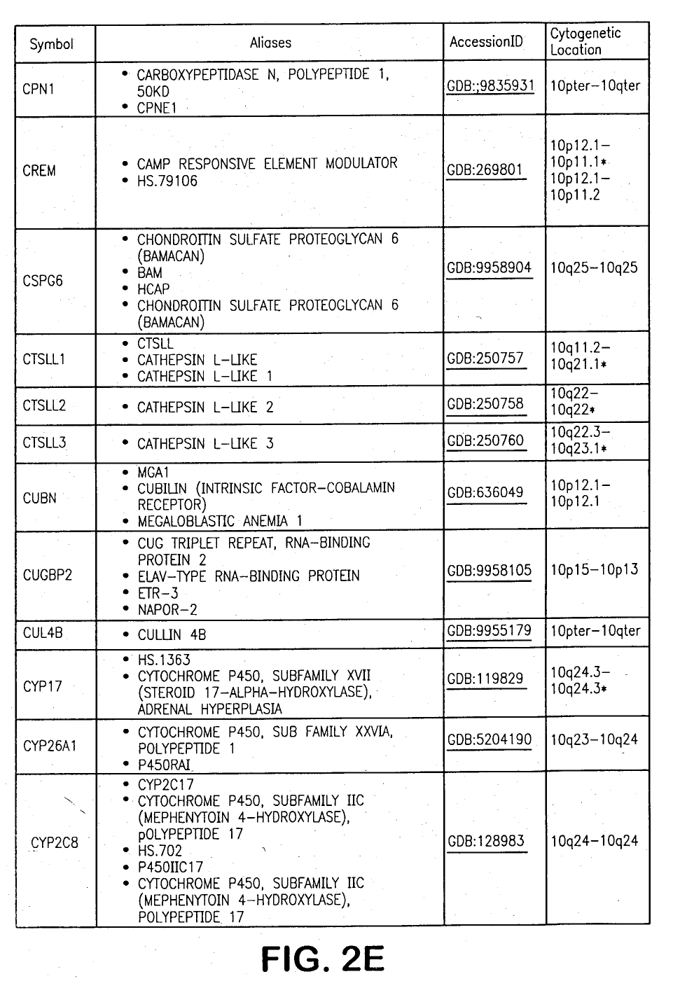

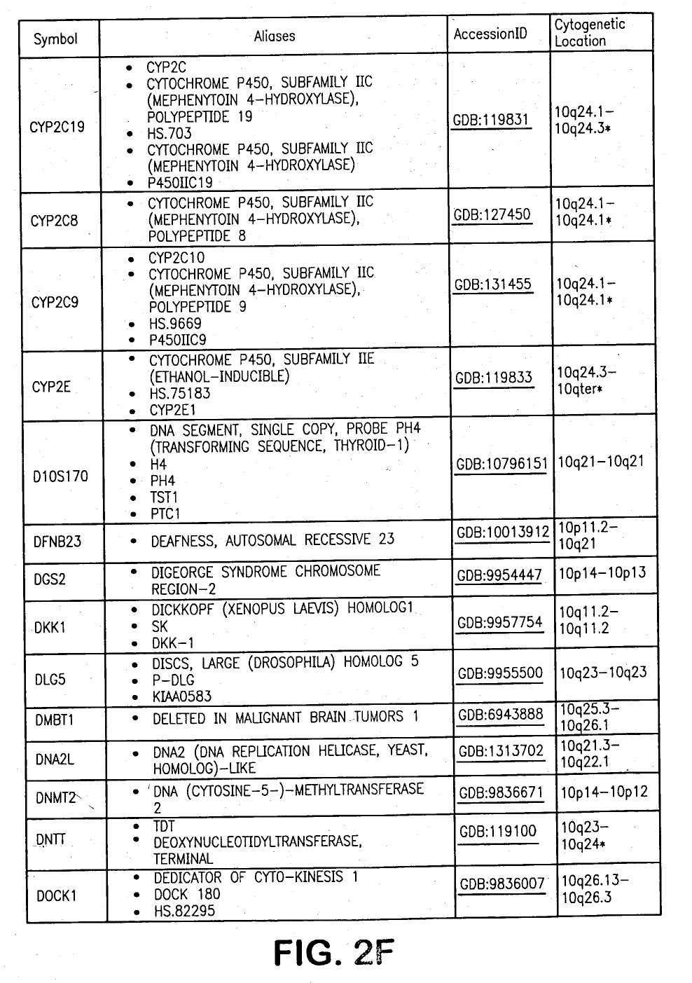

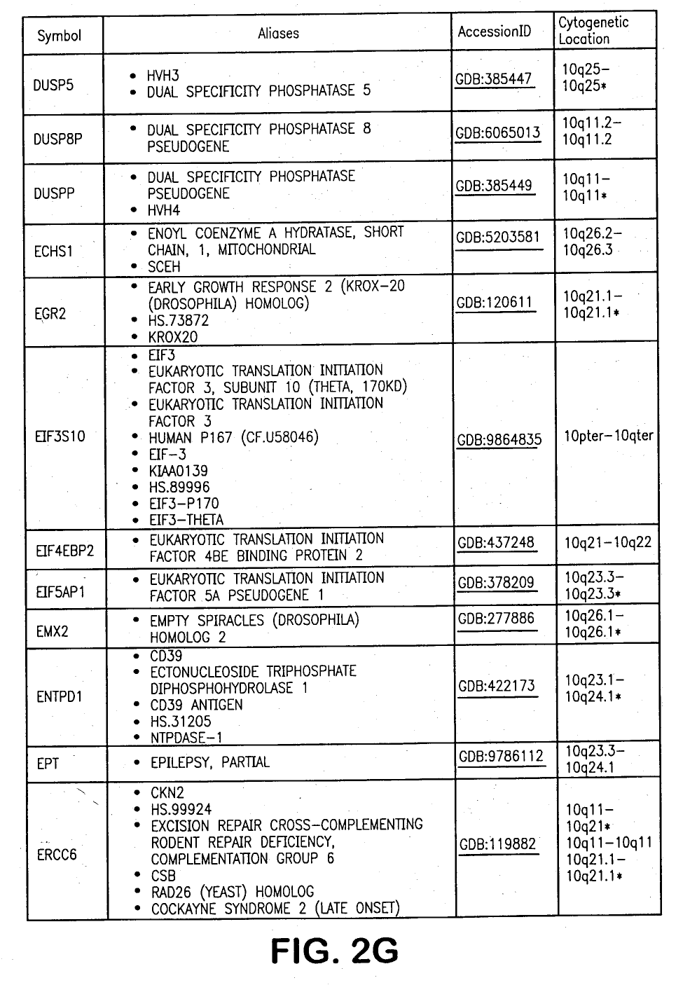

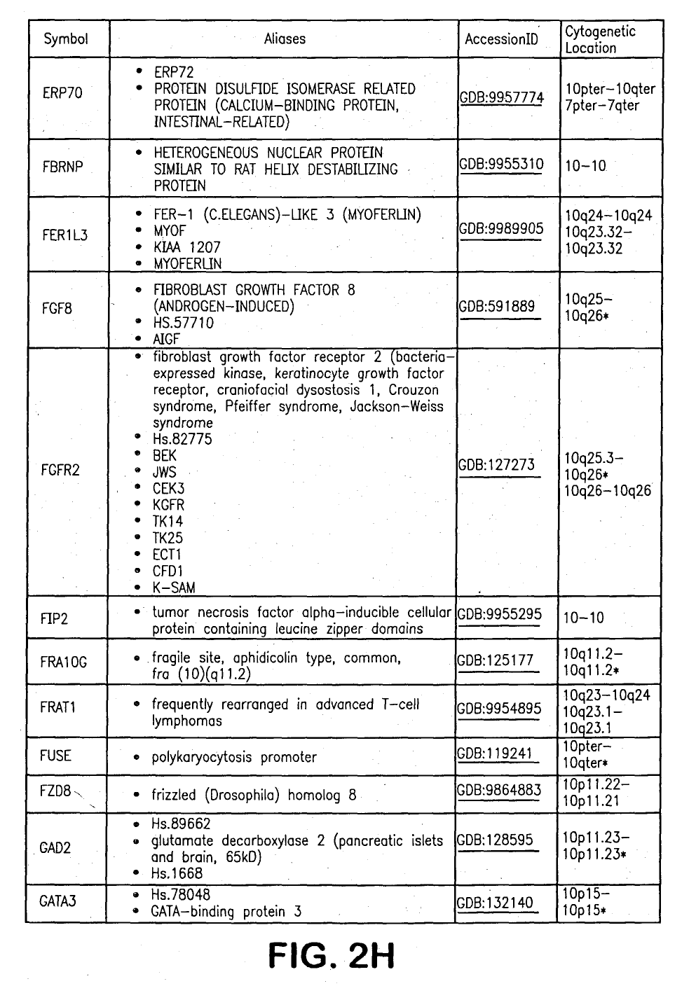

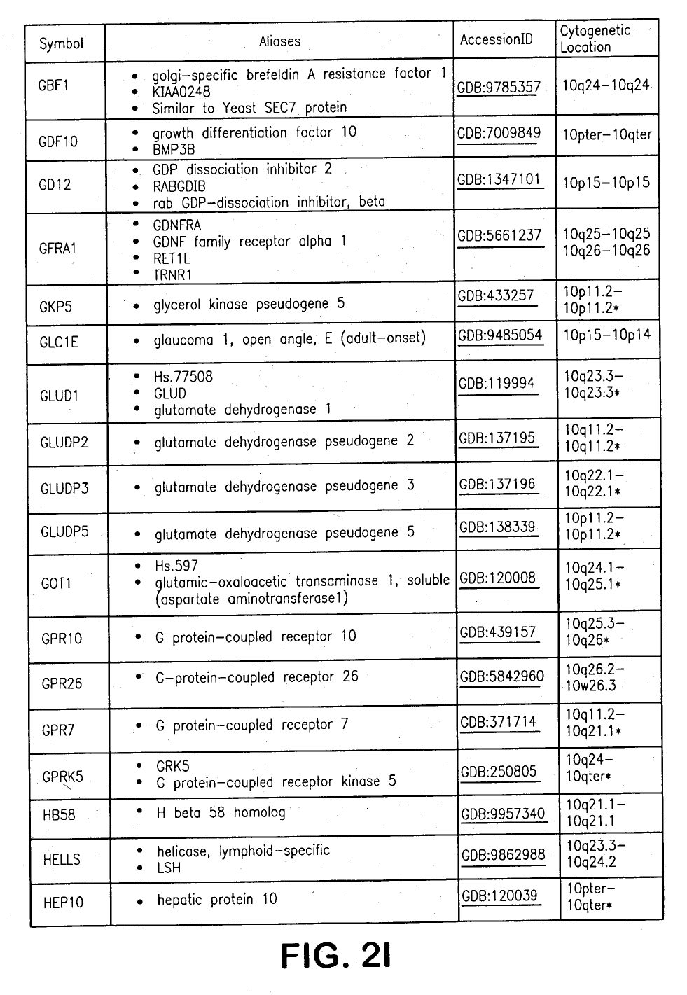

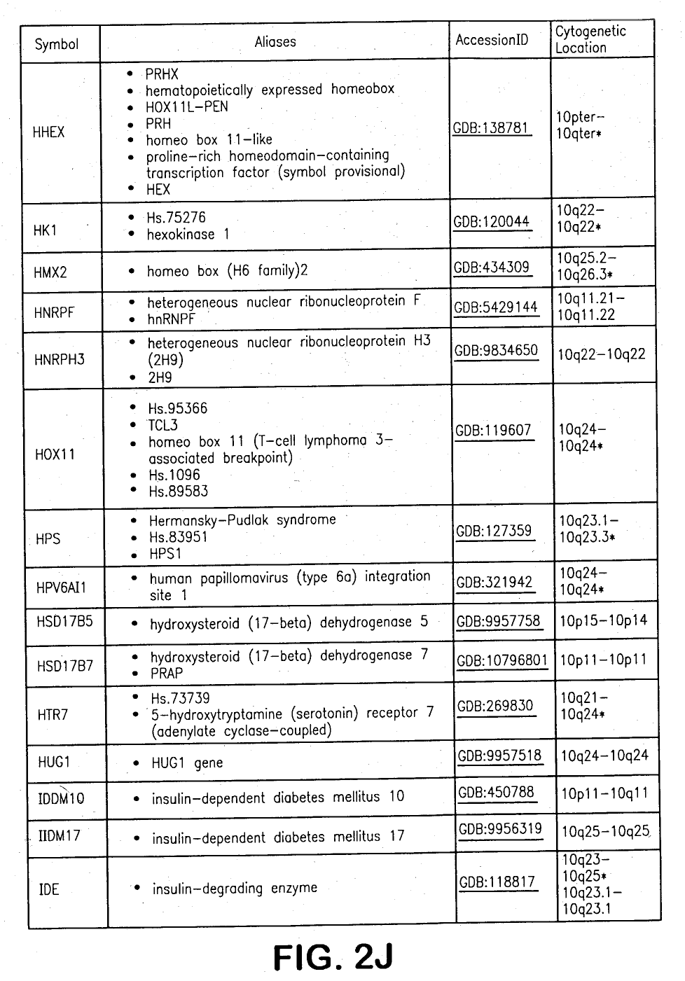

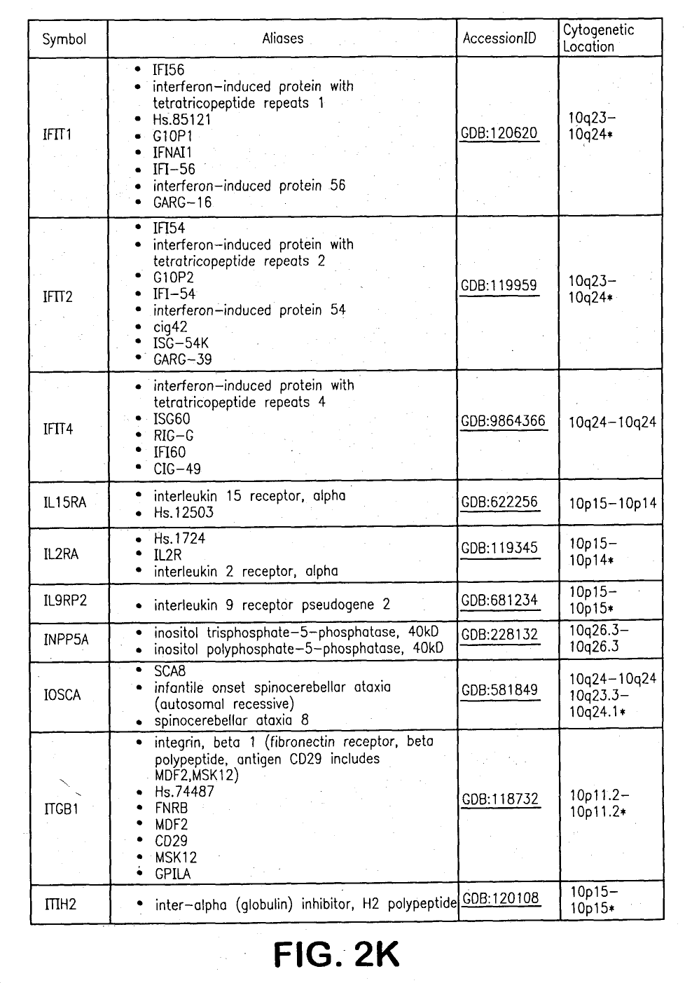

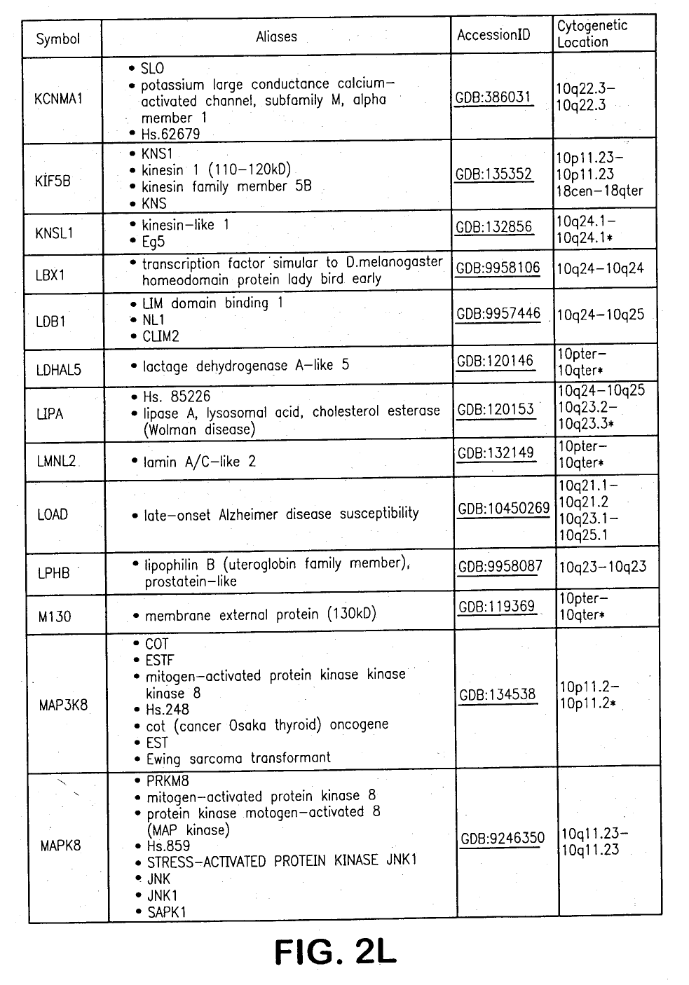

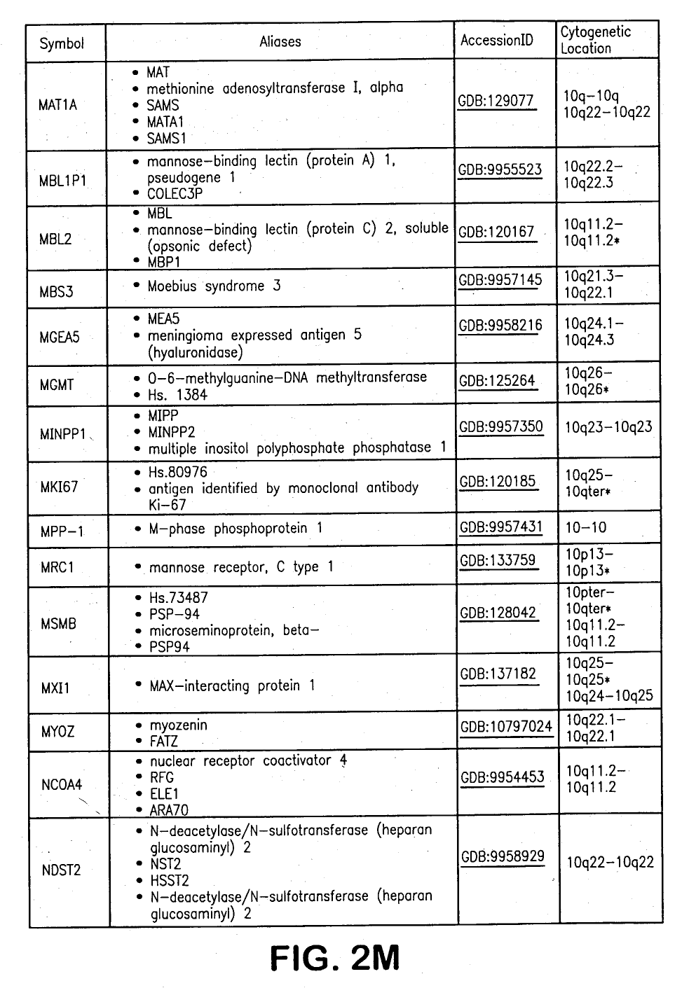

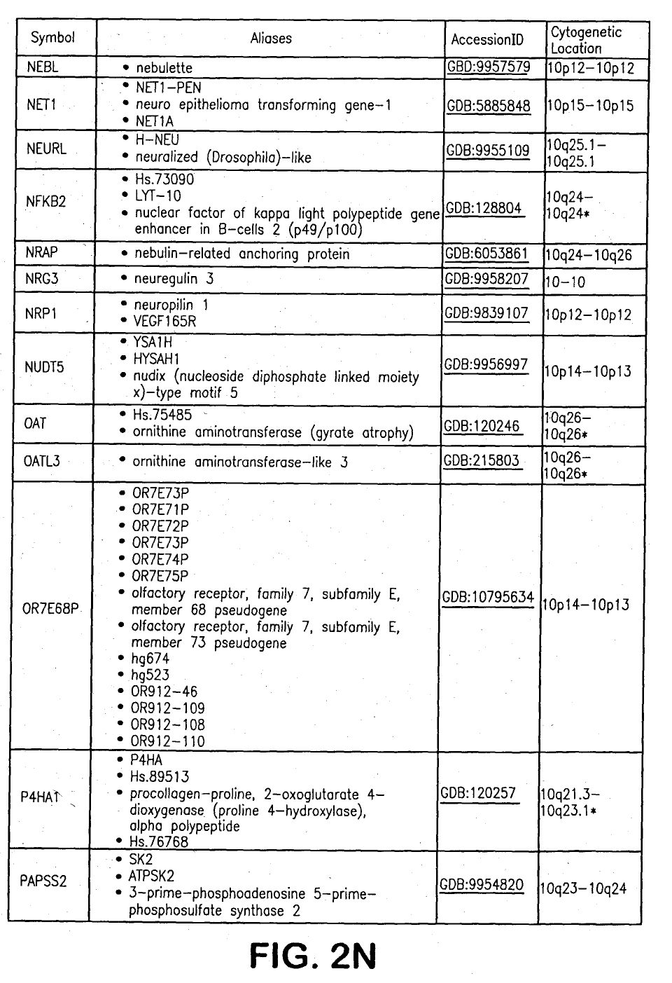

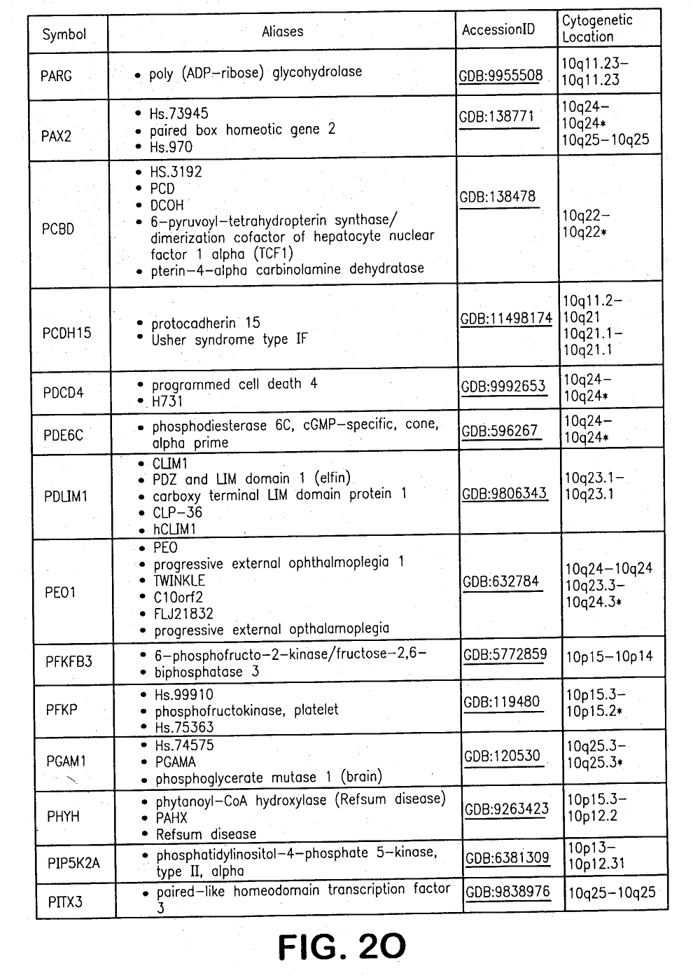

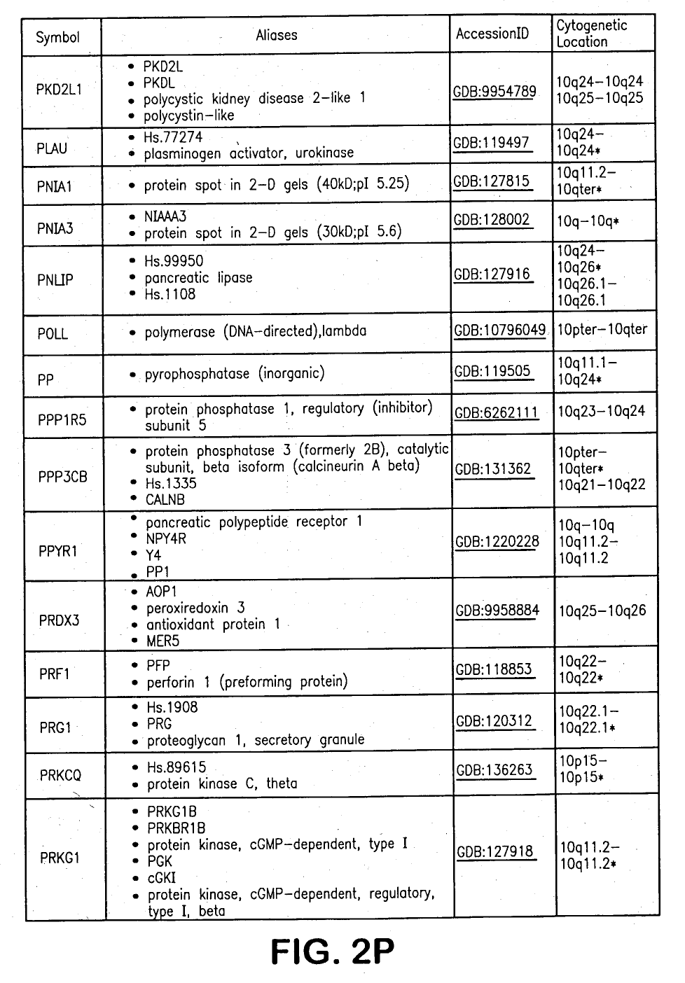

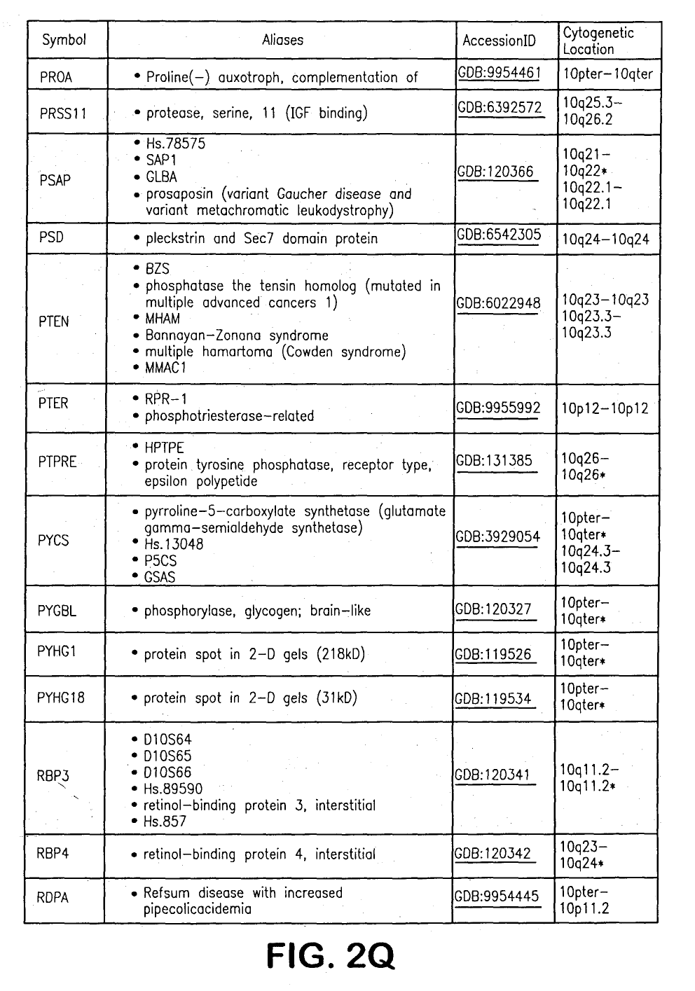

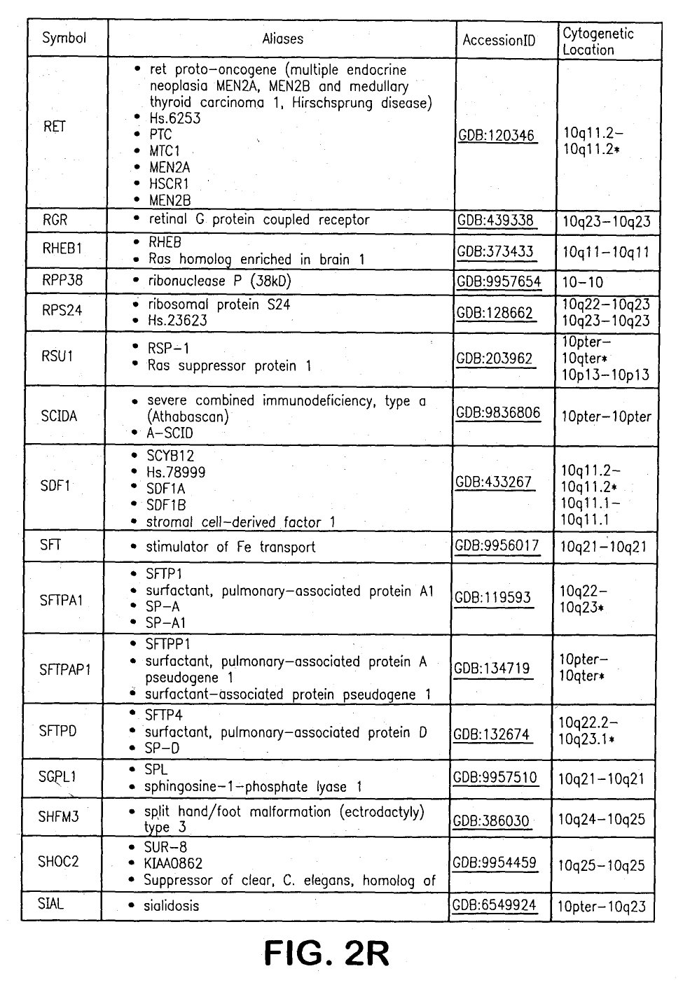

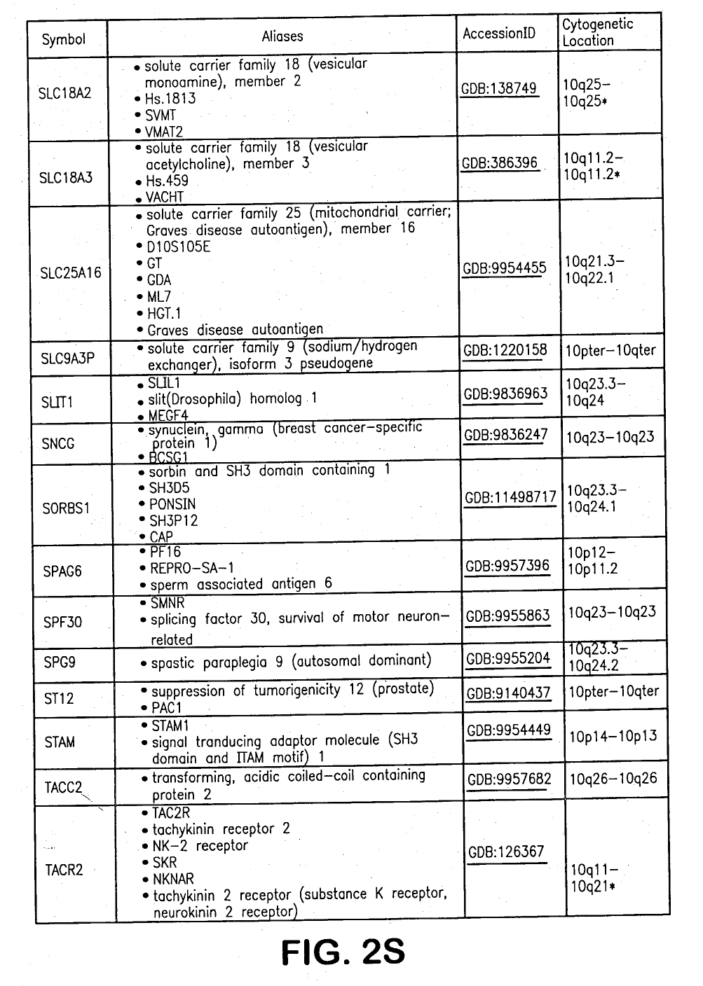

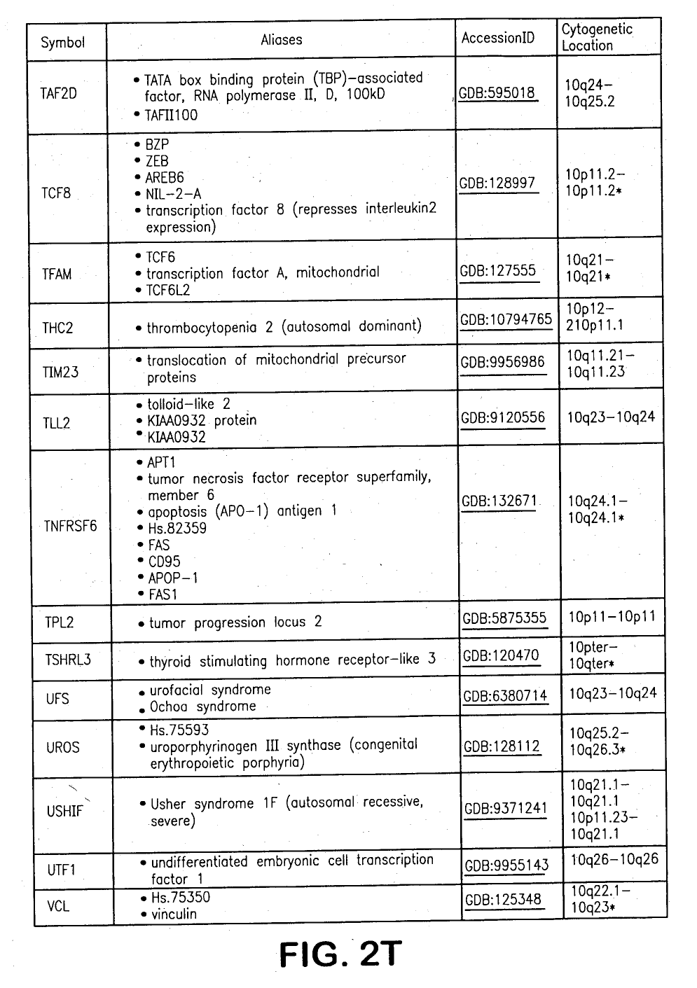

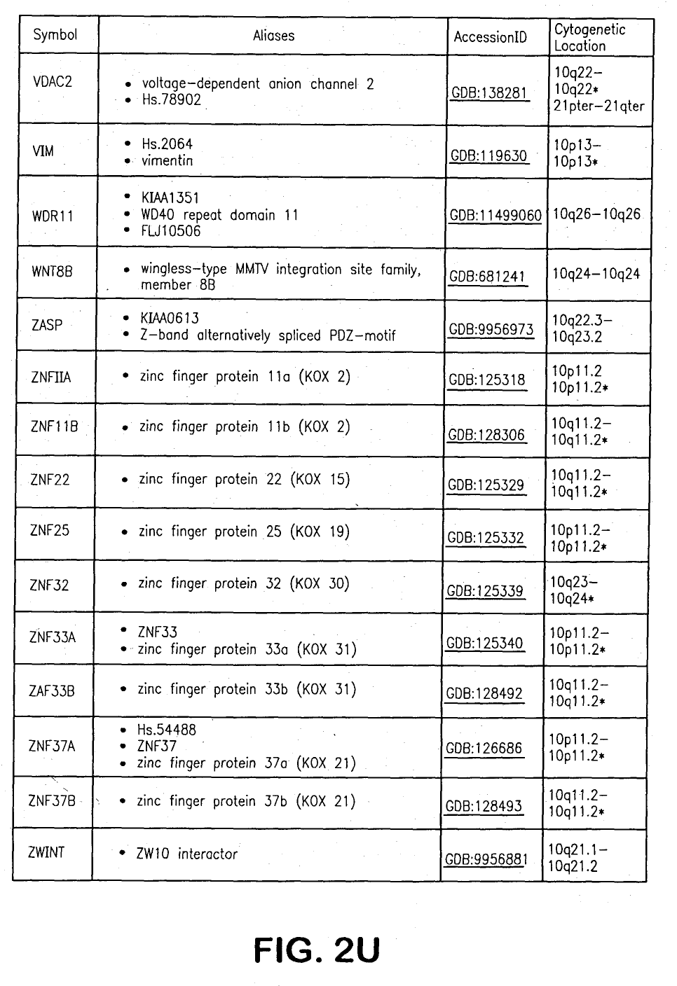

The methods of identifying a candidate AD gene include a step of selecting a gene on chromosome 10 that is or encodes a product that has one or more properties relating to one or more phenomena in neurodegenerative disease. Human chromosome 10 contains at least 600 genes. FIG. 2 provides a list of many of the genes that are located on chromosome 10. Additional genes that have been mapped to chromosome 10 are also known. Thus, genes on chromosome 10 may be evaluated as possible candidate AD genes on the basis of, for example, knowledge of the functions of the genes or products thereof and/or their occurrence or alteration in neurodegenerative diseases, and, in partcular, AD.

A particular method of identifying a candidate AD gene provided herein includes a step of selecting a gene on chromosome 10q that is or encodes a product that has properties relating to one or more phenomena in neurodegenerative disease. In further embodiments of these methods, the gene is on chromosome 10q22, 10q23, 10q24, 10q25 or 10q26. In particular embodiments of these methods, the gene is located within a region identified herein as containing or near markers associated with AD. In further embodiments, the gene is on chromosome 10q22, 10q23 or 10q24. In yet further embodiments, the gene is on chromosome 10q23, 10q24 or 10q25. The step of selecting a gene may also be a step of selecting a gene located in one of the following regions of chromosome 10: the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further particular embodiments of these methods, the gene is located in a region of chromosome 10 as follows: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583 (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

(2) Properties Relating to Phenomena in Neurodegenerative Disease

In the methods of identifying a candidate AD gene provided herein, a gene on chromosome 10, and, in particular embodiments, on particular regions of chromosome 10 as described above, is selected that is or encodes a product that has properties relating to one or more phenomena in neurodegenerative disease. The properties may be any aspect or feature of the gene or gene product, including but not limited to its physical composition (e.g., nucleic acids, amino acids, peptides and proteins), functional attributes (e.g., enzymatic capabilities, such as an enzyme catalyst, inhibitory functions, such as enzyme inhibition, antigenic properties, and binding capabilities, such as a receptor or ligand), cellular location(s), expression pattern (e.g., expression in the central nervous system and cells and tissues associated therewith) and/or interactions with other compositions.

The properties of the gene or gene product that are selected for in the methods of identifying a candidate AD gene are those that relate to one or more phenomena in neurodegenerative disease. Such phenomena, which have been widely described and are known to those of skill in the art, are numerous and include morphological, structural, biological and biochemical occurrences which can be pathophysiological aspects of neurodegenerative diseases. Such phenomena include, but are not limited to, senile plaques, neuritic plaques, and components of each, neurofibrillary tangles, tau protein and abnormal phosphorylation of tau protein, amyloid precursor protein (APP) and processing thereof, Aβ42 protein, α-, β- and γ-secretases, presenilin proteins, amyloid deposition, Lewy bodies, prions, apoptosis [see, e.g., Behl (2000) J. Neural Transm. 107:1325-1344], caspases, inflammation [see, e.g., McGeer amd McGeer (1998) Exp. Gerontol. 33:371-378], excitotoxicity and excitotoxins, excessive nitric oxide production, oxidative stress [see, e.g., Beal (1998) Biochim. Biophys. Acta Mol. Cell Res. 1366:211-223 and Wallace et al. (1998) Biofactors 7:187-190], proteases, protease inhibitors, neurotrophic factors, cytokines, calcium-dependent processes, signal transduction, altered ionic homeostasis, particularly calcium homeostasis, synaptic molecules, adhesion molecules, molecules involved in membrane turnover, cholesterol and lipid metabolism and transport, cytoskeletal molecules, neuronal and brain proteins, and cell necrosis. For example, a property of a gene or gene product relating to one or more phenomena in neurodegenerative disease may be involvement of the gene and/or product thereof in a pathway involved in APP metabolism, Aβ protein generation, aggregation and/or degradation, apoptosis, calcium homeostasis, inflammation, oxidative stress, free radical generation, modification of tau protein (e.g., phosphorylation), axonal transport, neuroprotection and neurotrophism. In particular embodiments of the methods of identifying a candidate AD gene provided herein, a gene on chromosome 10 is selected that is or encodes a product that has properties relating to one or more phenomena in Alzheimer's disease [see, e.g., Chapman et al (2001) Trends Genet. 17:254-261 and Selkoe (2001) Physiol. Rev. 81:741-766 for exemplary phenomena in AD].

Properties of the selected gene can relate to phenomena in neurodegenerative disease in many different ways. For example, a gene product may be involved in biological or biochemical processes that have been characterized as occurring in AD and/or other neurodegenerative or neurological diseases or disorders. Thus, for instance, a gene product may be an enzyme, inhibitor or regulatory subunit in a pathway leading to the generation of a component of an amyloid plaque, e.g., Aβ, alpha-1-anti-chymotrypsin, cathepsin D, non-amyloid component protein, apolipoprotein E (APOE), apolipoprotein J, heat shock protein 70, complement components, alpha2-macroglobin, interleukin-6, proteoglycans and serum amyloid P. A gene product may also be a molecule, e.g., an enzyme, involved in protein phosphorylation leading to hyperphosphorylation. A gene product may be a protein, such as a protein found in the brain, that is increased, decreased or altered in some other way in neurodegenerative diseases, such as, for example, AD. Thus, a property of a gene or gene product can relate to a phenomenon in neurodegenerative disease if it is directly or indirectly involved in, for example, a process or aspect characteristic of a neurodegenerative disease.

(3) Candidate AD Genes

Candidate AD genes are also provided herein. The candidate AD genes are located on human chromosome 10. In particular embodiments, the candidate AD gene is located on chromosome 10q. In further embodiments, the candidate AD gene is located on chromosome 10q22, 10q23, 10q24, 10q25 or 10q26. Candidate genes of particular embodiments, are located within a region identified herein as containing or near markers associated with AD. In further embodiments, the candidate AD gene is on chromosome 10q22, 10q23 or 10q24. In yet further embodiments, the candidate AD gene is on chromosome 10q23, 10q24 or 10q25. Candidate AD genes include genes located in the following regions of human chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

Candidate AD genes of particular interest are located in a region of chromosome 10 as follows: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In particular embodiments of the candidate AD genes provided herein, the gene is located within a region identified herein as containing or near markers associated with AD.

Particular candidate AD genes include genes listed in TABLE 1. Also listed in TABLE 1 are citations of publications which may be concerned with the gene and/or a product of the gene.

TABLE 1

| | Approximate | | | |
|---|---|---|---|---|
| | Location on | GenBank | Approximate | |
| | Chromosome | Accession | Cytogenetic | |
| Gene | 10 (bp) | No. | Location | Reference |
| SGPL1 | 77105083- | NT_024089^{a} | 10q22 | Van Veldhoven et |
| | 77166645 | XM_005931^{b} | | al., (2000) Biochim |
| | | XM_046325^{b} | | Biophys Acta, |
| | | XM_046326^{b} | | 1487(2-3):128-34 |
| PCBD | 77172724- | NT_024089^{a} | 10q22 | Milatovich et al., |
| | 77174993 | XM_046338^{b} | | (1993) Genomics |
| | | XM_046339^{b} | | 16(1):292-5 |
| | | | | Hauer et al., (1993) |
| | | | | J. Biol. Chem. |
| | | | | 268(7):4828-31 |
| FLJ11160 | 77611041- | NT_008849^{a} | 10q22.1 | |
| | 77651484 | XM_005936^{b} | | |
| | | XM_038317^{b} | | |
| PSAP | 77761345- | NT_028289^{a} | 10q22.1 | Morimoto et al., |
| | 77796272 | XM_045137^{b} | | (1989) Proc. Natl. |
| | | XM_045139^{b} | | Acad. Sci. USA |
| | | XM_045140^{b} | | 86(9):3389-93 |
| | | | | Rorman et. al., |
| | | | | (1989) Genomics |
| | | | | 5(3):486-92 |
| MYOZ | 78421968- | NT_024037^{a} | 10q22.1 | Takada et. al., |
| | 78432065 | XM_005876^{b} | | (2001) Proc. Natl. |
| | | | | Acad. Sci. USA |
| | | | | 98(4):1595-600 |
| FLJ12921 | 78456597- | NT_024037^{a} | 10q22.1 | |
| | 78462094 | XM_015246^{b} | | |
| LOC51008 | 78603718- | AF132952^{b} | 10q22.1 | Lai et. al., (2000) |
| | 78668872 | | | Genome Res. |
| | | | | 10(5):7103-13 |
| KIAA0275 | 78676384- | NT_028289^{a} | 10q22.1 | Nagase et. al., |
| | 78706358 | XM_005823^{b} | | (1996) DNA Res. |
| | | XM_045160^{b} | | 3(5):321-9 |
| CHST3 | 78756110- | NT_028289^{a} | 10q22.1 | Tsutsumi et. al., |
| | 78759658 | XM_011902^{b} | | (1998) FEBS Lett. |
| | | XM_045142^{b} | | 441(2):235-41 |
| | | | | Fukuta et. al., |
| | | | | (1998) Biochim. |
| | | | | Biophys Acta. |
| | | | | 1399(1):57-61 |
| CBARA1 | 78889122- | NT_024037^{a} | 10q22.2 | Wiemann et. al., |
| | 79102604 | XM_011886^{b} | | (2001) Genome |
| | | XM_032486^{b} | | Res. 11(3):422-35 |
| | | XM_032487^{b} | | Natter et. at., |
| | | XM_032489^{b} | | (1998) FASEB J. |
| | | | | 12(14):1559-69 |
| P4HA1 | 79554896- | NT_024037^{a} | 10q22.1 | Pajunen et. al., |
| | 79611864 | XM_005728^{b} | | (1989) Am. J. Hum. |
| | | XM_032511^{b} | | Genet. 45(6):829- |
| | | | | 34 |
| HSGT1 | 79704559- | NT_024037^{a} | 10q22.1 | Sato et. al., (1999) |
| | 79741363 | XM_032513^{b} | | Mol. Gen. Genet. |
| | | | | 260(6):535-40 |
| ANXA7 | 79927670- | NT_024037^{a} | 10q22.2 | Shirvan et. al., |
| | 79961935 | XM_005691^{b} | | (1994) Biochemistry |
| | | XM_032526^{b} | | 33(22):6888-901 |
| | | XM_032527^{b} | | |
| PPP3CB | 79995085- | NT_024037^{a} | 10q22.2 | Giri et. al., (1991) |
| | 80088187 | XM_011860^{b} | | Biochem. Biophys |
| | | XM_032530^{b} | | Res Commun. |
| | | | | 181(1):252-8 |
| PLAU | 80341334- | NT_024037^{a} | 10q22.2 | Nagai et. al., (1985) |
| | 80347653^{c} | XM_044353^{b} | | Gene 36(1-2):183-8 |
| | | XM_053443^{b} | | |
| | 80487562- | XM_053444^{b} | 10q22.2 | |
| | 80493880^{d} | XM_053445^{b} | | |
| | | XM_053446^{b} | | |
| | | XM_053447^{b} | | |
| VCL | 80347876- | NT_024037^{a} | 10q22.1-q23 | Weller et. al., |
| | 80674386 | XM_005774^{b} | | (1990) Proc. Natl. |
| | | XM_011883^{b} | | Acad. Sci. USA |
| | | | | 87(15):5667-71 |
| AP3M1 | 80580847- | NT_024037^{a} | 10q22.2 | Dell' Angelica et. al., |
| | 80637409 | XM_011882^{b} | | (1999) Mol. Cell. |
| | | XM_032482^{b} | | 3(1):11-21 |
| VDAC2 | 81694125- | NT_024037^{a} | 10q22 | Messina et. al., |
| | 81714428 | XM_005893^{b} | | (1999) Biochem. |
| | | XM_032437^{b} | | Biophys Res. |
| | | XM_032438^{b} | | Commun. |
| | | | | 255(3):707-10 |
| KGNMA1 | 83590532- | NT_008748^{a} | 10q22 | Meera, et. al., |
| | 84129270 | XM_005859^{b} | | (1997) Proc. Natl. |
| | | XM_039586^{b} | | Acad. Sci. USA |
| | | XM_039587^{b} | | 94(25):14066-71 |
| | | XM_039588^{b} | | |
| | | XM_039589^{b} | | |
| | | XM_039590^{b} | | |
| | | XM_039591^{b} | | |
| SNCG | 95002611- | AF037207^{a} | 10q23.2- | Lavedan et. al., |
| | 95007211 | AF044311^{a} | q23.3 | (1998) Hum. Genet. |
| | | AF010126^{b} | | 103(1):106-12 |
| | | AF017256^{b} | | |
| GLUD1 | 94865671- | NT_008793^{a} | 10q23.3 | Jung et. al., (1989) |
| | 95059882 | XM_005760^{b} | | Ann. Genet. |
| | | XM_010438^{b} | | 32(2):109-10 |
| | | XM_042914^{b} | | |
| | | XM_042915^{b} | | |
| | | XM_042916^{b} | | |
| TNFRSF6 | 96981296- | NT_008769^{a} | 10q24.1 | Lichter et. al., |
| | 97006412 | XM_048187^{b} | | (1992) Genomics |
| | | XM_048189^{b} | | 14(1):179-80 |
| | | XM_048190^{b} | | Inazawa et. al. |
| | | XM_048193^{b} | | (1992) Genomics |
| | | XM_048194^{b} | | 14(3):821-2 |
| LIPA | 97116874- | NT_008769^{a} | 10q23.2- | Anderson et. al., |
| | 97231924 | XM_048185^{b} | q23.3 | (1993) Genomics |
| | | | | 15(1):245-7 |
| CH25H | 97180588- | NT_008769^{a} | 10q23 | Lund et. al. (1998) |
| | 97181942 | XM_015669^{b} | | J. Biol. Chem. |
| | | | | 273(51):34316-27 |
| PPP1R3C | 99960732- | NT_008769^{a} | 10q23-q24 | Doherty et. al., |
| | 99963972 | XM_005884^{b} | | (1996) FEBS Lett. |
| | | | | 399(3):339-43 |
| KIAA0940 | 100379711- | NT_008679^{a} | 10q23.33 | Nagase et. al., |
| | 100574315 | XM_005886^{b} | | (1999) DNA Res. |
| | | | | 6(1):63-70 |
| FLJ20445 | 100622230- | NT_008769^{a} | 10q23.33 | |
| | 100682998 | XM_005885^{b} | | |
| IDE | 100809864- | NT_008769^{a} | 10q23-q25 | Affholter et. al., |
| | 100929731 | XM_005890^{b} | | (1990) Mol. |
| | | XM_051153^{b} | | Endocrinol. |
| | | | | 4(8):1125-35 |
| KNSL1 | 100948951- | NT_008769^{a} | 10q24.1 | Tihy et. al., (1992) |
| | 101011338 | XM_005889^{b} | | Genomics |
| | | XM_051151^{b} | | 13(4):1371-2 |
| | | XM_051152^{b} | | |
| FER1L3 | 101784450- | NT_008769^{a} | 10q24 | Britton et. al., |
| | 101959700 | XM_011919^{b} | | (2000) Genomics |
| | | XM_051131^{b} | | 68(3):313-21 |
| | | XM_051132^{b} | | |
| LGI1 | 102235341- | NT_008769^{a} | 10q24 | Chernova et. al., |
| | 102272202 | XM_051122^{b} | | (1998) Oncogene |
| | | | | 17(22):2873-81 |
| TLL2 | 104847041- | NT_029394^{a} | 10q23-q24 | Scott et. al., (1999) |
| | 104994622 | XM_005684^{b} | | cytogenet. Cell |
| | | XM_050998^{b} | | Genet. 86(1):64-5 |
| BTRC | 109955041- | AF101784^{b} | 10q24-q25 | Fujiwara et. al., |
| | 110133992 | AF129530^{b} | | (1999) Genomics |
| | | Y14153^{b} | | 58(1):104-5 |
| SLIT1 | 105478243- | NT_029384^{a} | 10q23.3-q24 | Itoh et. al., (1998) |
| | 105662868 | XM_005958^{b} | | Brain Res. Mol. |
| | | XM_050940^{b} | | Brain Res. |
| | | | | 62(2):175-86 |
| PI4KII | 106223247- | NT_029377^{a} | 10q24 | Minoque et. al., |
| | 106244559 | XM_050848^{b} | | (2001) J. Biol. |
| | | XM_050849^{b} | | Chem. |
| | | XM_050850^{b} | | 6(20):16635-40 |
| SFRP5 | 106288513- | NT_029377^{a} | 10q24.1 | Chang et. al., |
| | 106291592 | XM_050823^{b} | | (1999) Hum. Mol. |
| | | XM_050824^{b} | | Genet. 8(4):575-83 |
| CPN1 | 108627055- | NT_008635^{a} | 10q24.2 | Riley et. al., (1998) |
| | 108676087 | XM_011899^{b} | | Genomics |
| | | | | 50(1):105-8 |
| KEO4 | 108730049- | NT_008635^{a} | 10q24.2 | Li et. al., (2000) |
| | 108763721 | XM_011898^{b} | | Biochem. Biophys. |
| | | | | Res. Commun. |
| | | | | 279(2):487-93 |
| CHUK | 108767286- | NT_008635^{a} | 10q24-q25 | Mock et. al., (1995) |
| | 108989876 | XM_030458^{b} | | Genomics |
| | | XM_030459^{b} | | 27(2):348-51 |
| WNT8B | 109222612- | NT_029383^{a} | 10q24 | Lako et. al., (1996) |
| | 109243315 | XM_005702^{b} | | Genomics |
| | | | | 35(2):386-8 |
| NPM3 | 110432000- | AF079325^{a} | 10q24.32 | MacArthur & |
| | 110434076 | AF081280^{b} | | Shackleford (1997 |
| | | | | Genomics |
| | | | | 42(1):137-40 |
| KCNIP2 | 110478028- | NT_008804^{a} | 10q24 | An et. al., (2000) |
| | 110494243 | XM_005926^{b} | | Nature |
| | | XM_031869^{b} | | 403(6769):553-6 |
| | | XM_031870^{b} | | |
| | | XM_031871^{b} | | |
| | | XM_031872^{b} | | |
| | | XM_031873^{b} | | |
| | | XM_031874^{b} | | |
| | | XM_031875^{b} | | |
| | | XM_031876^{b} | | |
| | | XM_031878^{b} | | |
| KIAA0595 | 110796501- | NT_008804^{a} | 10q24.32 | Nagase et. al., |
| | 110805189 | XM_016112^{b} | | (1998) DNA Res. |
| | | XM_031886^{b} | | 5(1):31-9 |
| | | XM_031887^{b} | | |
| | | XM_031888^{b} | | |
| GBF1 | 110900561- | NT_008804^{a} | 10q24 | Mansour et. al., |
| | 111064179 | XM_005923^{b} | | (1998) Genomics |
| | | XM_031908^{b} | | 54(2):323-7 |
| | | XM_031909^{b} | | |
| | | XM_031910^{b} | | |
| | | XM_031911^{b} | | |
| | | XM_031912^{b} | | |
| NFKB2 | 111076993- | NT_008804^{a} | 10q24 | Liptay et. al., |
| | 111080520 | XM_031926^{b} | | (1992) Genomics |
| | | XM_031927^{b} | | 13(2):287-92 |
| | | XM_031928^{b} | | |
| | | XM_031929^{b} | | |
| PSD | 111083905- | NT_008804^{a} | 10q24 | Perletti et. al., |
| | 111098162 | XM_005921^{b} | | (1997) Genomics |
| | | | | 46(2):251-9 |
| NEURL | 112578425- | NT_029393^{a} | 10q25.1 | Nakamura et. al., |
| | 112674891 | XM_005941^{b} | | (1998) Oncogene |
| | | XM_032003^{b} | | 16(8):1009-19 |
| DUSP5 | 120352413- | NT_008669^{a} | 10q25 | Martell et. al., |
| | 120366032 | XM_005756^{b} | | (1994) Genomics |
| | | XM_045322^{b} | | 22(2):462-4 |
| PDCD4 | 120723116- | NT_008669^{a} | 10q24 | Soejima et. al., |
| | 120745367 | XM_005698^{b} | | (1999) Cytogenet |
| | | | | Cell Genet 87(1- |
| | | | | 2):113-4 |
| ADRA2A | 120968570- | M18415^{a} | 10q24-q26 | Hoehe et. al., |
| | 120969922 | M23533^{a} | | (1995) J. Mol. Med. |
| | | | | 73(6):299-306 |
| SLC18A2 | 127886458- | NT_029396^{a} | 10q25 | Surratt et. al., |
| | 127922683 | XM_005978^{b} | | (1993) FEBS Lett. |
| | | XM_048899^{b} | | 318(3):325-30 |
| | | XM_048900^{b} | | |
| | | XM_048901^{b} | | |
| PRDX3 | 129883719- | NT_008902^{a} | 10q25-q26 | Tsuji et. al., (1995) |
| | 129894799 | XM_005968^{b} | | Biochem. J. |
| | | | | 307(pt2):377-81 |
| EMX2 | 128188763- | NT_029396^{a} | 10q26.1 | Katsury et. al., |
| | 128193445 | XM_049756^{b} | | (1994) Genomics |
| | | | | 22(1):41-5 |
| GPRK5 | 129962307- | NT_008902^{a} | 10q24-qter | Bullrich et. al., |
| | 130217320 | XM_005969^{b} | | (1995) Cytogenet. |
| | | | | Cell Genet. 70(3- |
| | | | | 4):250-4 |
| DOCK1 | 137159760- | NT_028285^{a} | 10q26.13- | Hasegawa et al., |
| | 137637128 | XM_028933^{b} | q26.3 | (1996) Mol. Cell. |
| | | | | Biol. 16(4):1770-6 |

^{a}GenBank accession number for chromosomal nucleotide sequence containing the gene.
^{b}GenBank accession number for mRNA sequence encoded by the gene.
^{c}One of two or more approximate locations on chromosome 10 according to the UCSC Golden Path Genome Browser August 2001 Freeze (http://genome.ucsc.edu/index.html).
^{d}One of two or more approximate locations on chromosome 10 according to the UCSC Golden Path Genome Browser August 2001 Freeze (http://genome.ucsc.edu/index.html).

Particular candidate AD genes provided herein include SNCG (encoding γ-synuclein or persyn), TNFRSF6 (encoding tumor necrosis factor receptor superfamily member 6 or FAS), LIPA (encoding lipase A), IDE (encoding insulin degrading enzyme), KNSL1 (encoding kinesin-like 1), PLAU (urokinase plasminogen activator), TLL2 (tolloid-like 2), PSAP (prosaposin), PSD (pleckstrin and Sec7 domain protein), KIAA0904, NFKB2 (nuclear factor of kappa light polypeptide gene enhancer in B-cells 2, p49/p100), PPP3CB (protein phosphatase 3, catalytic subunit, beta isoform, calcineurin A beta), CH25H (cholesterol 25-hydroxylase) and FERIL3 (C. elegans-like 3, myoferlin).

(4) Methods for Identifying an AD DNA Segment or AD Gene

In a method provided herein for identifying an AD DNA segment or an AD gene utilizing a candidate gene approach, one step of the method may be selecting a candidate AD gene on chromosome 10 such as described above. The candidate AD gene may be one that is or encodes a product that has properties relating to one or more phenomena in neurodegenerative disease. Particular embodiments of the methods provided herein for identifying an AD DNA segment or gene include a step of selecting a candidate AD gene on chromosome 10q. In further embodiments of these methods, the methods include a step of selecting a candidate AD gene on chromosome 10q22, 10q23, 10q24, 10q25 or 10q26. In particular embodiments, the methods include a step of selecting a candidate gene on chromosome 10q22, 10q23 or 10q24. In yet further embodiments, the methods include a step of selecting a candidate gene on chromosome 10q23, 10q24 or 10q25.

In another embodiment of the methods provided herein for identifying an AD DNA segment or an AD gene utilizing a candidate gene approach, a step of the method may be selecting a candidate AD gene located within a region of chromosome 10 identified herein as containing or near a genetic marker associated with AD.

The methods provided herein for identifying an AD segment or an AD gene utilizing a candidate gene approach may further include a step of analyzing genetic markers on chromosome 10 located in or around a selected candidate gene for linkage and/or association with AD. A possible benefit in utilizing linkage analysis methods for assessment of candidate genes is that only a few polymorphic microsatellite markers in the candidate gene region may be required for the analysis. Microsatellite markers within the gene region are selected, and DNA from AD family samples is genotyped with respect to those markers. Parametric and/or nonparametric linkage analyses of the genotype data may be conducted as described herein or using methods known in the art. If positive results are obtained in the linkage analyses (e.g., a positive LOD score), they are indicative of linkage of the DNA segment to a DNA segment associated with AD. The LOD score values for markers within the candidate gene region can be compared to determine if they delineate a region in which an AD gene may be located. Additional markers at smaller genetic distances within an identified region may then be analyzed for linkage to AD in order to narrow in on the location of an AD gene. The markers may also be assessed through association analyses to ascertain whether any might lie within linkage disequilibrium range of an underlying AD DNA segment or gene.

The effectiveness of the candidate gene approach is influenced by the type and density of the markers utilized in the study as well as by study design and sample size. Although linkage analysis of candidate genes may utilize a relatively small number of microsatellite markers in the gene region, association analysis generally requires a fairly dense (on the order of kilobases) spacing of markers in the gene region. Thus, SNPs may be more suitable for such association analyses than microsatellite markers. Therefore, candidate gene approaches can typically involve sequence analysis of candidate genes, particularly of individuals from affected families, to identify useful markers in the genes.

b. Genetic Localization Approach

Genetic localization approaches do not require knowledge of the biological or biochemical nature of the disease. In contrast to a full candidate gene approach, which immediately restricts genetic analysis of a chromosome to a specific gene region determined by a hypothesis based on trait biology, genetic localization approaches first identify a chromosomal region in which a disease gene or DNA segment is located and then gradually reduce the size of the region in order to determine the location of the specific defective DNA segment as precisely as possible.

For example, in these methods, the position of an AD DNA segment or gene may be localized by determining LOD scores for different markers on chromosome 10. In particular embodiments of these methods the markers analyzed are located in the region of 10q22, 10q23, 10q24, 10q25 or 10q26. In further embodiments of these methods, the markers analyzed are located within a region identified herein as containing or near markers associated with AD. In particular embodiments, the markers analyzed are on chromosome 10q22, 10q23 or 10q24 or are on chromosome 10q23, 10q24 or 10q25. In further particular embodiments of these methods, the markers analyzed are located in: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further particular embodiments of these methods the markers analyzed for LOD scores are located in a region of chromosome 10 as follows: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

The strategy underlying this method is to select successive markers progressively closer to an AD gene, each marker being chosen based on the linkage distance established for the previous marker. Linkage distance is the distance from a marker at which the LOD score of the marker from an AD gene is maximized. For example, if a first marker gives a maximum LOD score at a linkage distance of 10 cM, a second marker would be selected within a 20 cM segment centered about the first marker. The linkage distance of the second marker to the gene is then determined. If this linkage distance is less than that of the first polymorphic marker, a further marker is selected within the linkage distance of the second marker. If the linkage distance of the second marker is greater than that of the first marker, then a further marker is selected within the linkage distance of the first marker, on the side distal from the second marker. By continually identifying markers progressively closer to the gene, it is possible to localize the position of the gene to a relatively small segment of DNA for which more detailed molecular studies are feasible.

Linkage tests can be powerful and specific for gene discovery; however, localization of the disease locus can be achieved only to a certain level of precision (i.e., on the order of many megabases) which represents a region that potentially can include hundreds of genes. Additionally, genes with small or subtle effects may not be detectable by linkage at all. Linkage studies are resource intensive and dependent on the availability of large family collections, which are not a requirement of most association-based methods of gene discovery.

2. Association Analysis in the Identification of AD DNA Segments or Genes

Studies based on pedigrees in many cases, particularly based on study design, may serve to only identify a chromosome that harbors a trait-causing allele and may possibly also serve to broadly outline an expansive region in which such an allele may reside within a chromosome. In contrast, analyses of genetic association can be a powerful method for fine-scale mapping of a chromosome thereby refining the region in which a trait-causing allele is located and even identifying the position of the allele.

There are, however, several situations which can lead to the detection of allelic association between loci that are in fact unlinked, such as, for example, population stratification. For this reason, linkage disequilibrium-based mapping is often used to fine-map a disease gene when a possible region of interest has already been identified. Thus, linkage disequilibrium-based gene discovery methods are usually used to define the chromosomal region containing a disease gene once linkage has been demonstrated for a chromosome.

Generally, when disequilibrium is suspected, affected individuals are checked for increased frequency of specific alleles for the marker loci. An excess frequency of any allele, as measured against general population frequencies (using the chi-square statistics) can indicate linkage disequilibrium.

b. Candidate Gene Approach

Generally, in a candidate gene approach to the identification of a disease gene using association analysis of polymorphic markers, one or a few markers around or within candidate disease genes, particularly those with hypothesized functional importance, are genotyped in a few hundred case and control individuals. The specific characteristics of the associated allele with respect to a candidate gene function usually give further insight into the relationship between the associated allele and the trait (causal or in linkage disequilibrium). If the evidence indicates that the associated allele within the candidate gene is most probably not the trait-causing allele but is in linkage disequilibrium with the real trait-causing allele, then the trait-causing allele can be found by sequencing the vicinity of the associated marker, and performing further association studies with the polymorphisms that are revealed in an iterative manner.

In order for association analysis to be useful, a dense map of markers is generally required since associations typically are found over shorter distances. It is estimated that SNPs occur, on average, every 1,000 bp and have a low mutation rate, both of which are characteristics that may have particular advantages for association analysis. Tens of thousands of known SNPs are located on chromosome 10, with an estimated mean intermarker distance of about 2 kb, and may be used in methods provided herein for identifying an AD segment or an AD gene utilizing a candidate gene approach.

As described above, in a method provided herein for identifying an AD DNA segment or an AD gene utilizing a candidate gene approach, one step of the method may be selecting a candidate AD gene on chromosome 10. The candidate AD gene may be one that is or encodes a product that has properties relating to one or more phenomena in neurodegenerative disease. Particular embodiments of the methods provided herein for identifying an AD DNA segment or gene include a step of selecting a candidate AD gene on chromosome 10q. In further particular embodiments of these methods, the selected candidate AD gene is on chromosome 10q22, 10q23, 10q24, 10q25 or 10q26. In particular embodiments, the selected candidate gene is on chromosome 10q22, 10q23 or 10q24 or on chromosome 10q23, 10q24 or 10q25.

In another embodiment of the methods provided herein for identifying an AD DNA segment or an AD gene utilizing a candidate gene approach, a step of the method may be selecting a candidate AD gene located within regions of chromosome 10 identified herein as containing or near genetic markers associated with AD.

The methods provided herein for identifying an AD segment or an AD gene utilizing a candidate gene approach may further include a step of analyzing genetic markers on chromosome 10 located in or around a selected candidate gene for association with AD. Any such chromosome 10 markers may be used in the methods. In particular embodiments of these methods, the markers analyzed are chromosome 10 markers described herein that are linked to one or more DNA segments, e.g., genes, associated with AD and/or are associated with AD. In further particular embodiments of these methods, the markers analyzed are SNPs. The methods provided herein for identifying an AD segment or an AD gene utilizing a candidate gene approach may include a step of conducting nucleotide sequence analysis of the candidate gene to identify polymorphic markers, and in particular SNPs, which are analyzed for association and/or linkage with AD.

Association studies may be conducted, for example, in two successive steps. In a first phase, the frequencies of a reduced number of markers from the candidate gene are determined in the trait-positive and control populations. In a second phase of the analysis, the position of the genetic locus responsible for the given trait is further refined using a higher density of markers from the relevant region. However, if the candidate gene under study is relatively small in length, a single phase may be sufficient to establish significant associations.

c. Genetic Localization Approach

Association analyses may be used in genetic localization approaches to disease gene discovery. In these methods, a region of a chromosome is first identified as being in proximity to a disease susceptibility gene by virtue of linkage disequilibrium detected as association between a highly informative marker in the region and a disease trait. In particular embodiments of these methods, the region identified is both linked and associated with disease. The identified region is then analyzed in finer detail through analysis of additional markers in the region for association with the disease trait. The application of haplotypes to this analysis may provide stronger evidence of a region being associated with the disease phenotype than does the use of individual markers.

For example, in these methods, the position of an AD gene may be localized by conducting AD association analyses on different markers on chromosome 10, individually or in combinations. In particular embodiments of these methods, the markers analyzed are on chromosome 10q. In further embodiments of this method, the markers analyzed are located in the region of 10q22, 10q23, 10q24, 10q25 or 10q26. In further embodiments of these methods, the markers analyzed are located within a region identified herein as containing or near markers associated with AD. In particular embodiments, the markers analyzed are on chromosome 10q22, 10q23 or 10q24 or on chromosome 10q23, 10q24 or 10q25. In still further embodiments of these methods, the markers analyzed for association with AD are located in: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

Markers of particular interest are located in a region of chromosome 10 as follows: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In particular embodiments of the association study-based disease gene localization methods provided herein, markers analyzed for association are located within a region identified herein as containing or near markers associated with AD.

3. Haplotype Analysis

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: the disease mutation is found only in the presence of a specific set of marker alleles. Through subsequent generations, recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are farther away. When not broken up by recombination, “ancestral” haplotypes and linkage disequilibrium between marker alleles at different loci can be tracked not only through pedigrees but also through populations.

A haplotype can be tracked through populations and its statistical association with a given trait can be analyzed. Complementing single point (allelic) association studies with multi-point association studies, also called haplotype studies, increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of markers can be determined. The haplotype frequency is then compared for distinct populations of trait positive and control individuals. The number of trait positive individuals, which should be subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found, the relative risk for an individual carrying the given haplotype of being affected with the trait under study can be approximated.

a. Determination of Haplotype Frequencies

When genotypes are determined, it is often not possible to distinguish heterozygotes so that haplotype frequencies cannot be easily inferred. When the gametic phase is not known, single chromosomes can be studied independently, for example, by asymmetric PCR amplification (see Newton et al. (1989) Nucleic Acids Res. 17:2503-2516; Wu et al. (1989) Proc. Natl. Acad. Sci. U.S.A. 86:2757), or by isolation of single chromosome by limit dilution followed by PCR amplification (see Ruano et al. (1990) Proc. Natl. Acad. Sci. U.S.A. 86:9079-9083). Further, a sample may be haplotyped for sufficiently close markers by double PCR amplification of specific alleles (Sarkar, G. and Sommer S. S. (1991) Biotechniques). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G. (1990) Mol. Biol. Evol 7:111-122 may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site.

Alternatively, haplotype frequencies can be estimated from the multilocus genotypic data. Any method known to person skilled in the art can be used to estimate haplotype frequencies [see, e.g., Lange (1997) Mathematical and Statistical Methods for Genetic Analysis, Springer, N.Y.; Weir (1996) Genetic Data Analysis II: Methods for Discrete Population Genetic Data, Sinauer Assoc., Inc., Sunderland, Mass., U.S.A.]. For example, maximum likelihood haplotype frequencies can be computed using an Expectation-Maximization (EM) algorithm [see, e.g., Dempster et al. (1977) J. R. Stat. Soc. 39B:1-38; Excoffier and Slatkin (1995) Mol. Biol. Evol. 12:921-927]. This procedure is an iterative process aiming at obtaining maximum likelihood estimates of haplotype frequencies from multi-locus genotype data when the gametic phase is unknown. Haplotype estimations are usually performed by applying the EM algorithm using for example the EM-HAPLO program [Hawley et al. (1994) Am. J. Phys. Anthropol. 18:104] or the Arlequin program [Schneider et al. (1997) Arlequin: A Software for Population Genetics Data Analysis, Univ. of Geneva]. The EM algorithm is a generalized iterative maximum likelihood approach.

To ensure that the estimation finally obtained is the maximum-likelihood estimation, several values of departures are required. The estimations obtained are compared, and, if they are different, the estimations leading to the best likelihood are kept.

Estimating the frequency of a haplotype for a set of polymorphisms in a population, can be carried out by: 1) genotyping at least one polymorphism for each individual in a population; 2) genotyping a second polymorphism by determining the identity of the nucleotides at the location of the polymorphism for both copies of the second polymorphism present in the genome of each individual in the population; and c) applying a haplotype determination method to the identities of the nucleotides determined in steps a) and b) to obtain an estimate of the frequency. Methods of estimating the frequency of a haplotype encompass methods used alone or in any combination and all others methods known to those of skill in the art in addition to those described herein.

b. Haplotype Analysis in the Identification of AD DNA Segments or Genes

Methods used for analyses of genetic association of individual markers with disease in the identification of AD DNA segments can likewise be applied to the analysis of genetic association of haplotypes of the markers with disease for the same purpose. Haplotype analysis can be a most powerful method for fine-scale mapping of a chromosome thereby refining the region in which a trait-causing allele is located and even identifying the position of the allele.

Provided herein are methods of detecting an association between a haplotype and AD. The methods include steps of: (a) estimating the frequency of at least one haplotype in a trait positive population, (b) estimating the frequency of the haplotype in a control population, and (c) determining whether a statistically significant association exists between the haplotype and AD.

The haplotypes used in these methods include combinations of markers provided herein. In particular embodiments of these methods, the markers analyzed in combinations as haplotypes are on chromosome 10q. In further embodiments of this method, the markers analyzed in combinations as haplotypes are located in the region of 10q22, 10q23, 10q24, 10q25 or 10q26. In further embodiments of these methods, the markers analyzed in combinations as haplotypes are located within a region identified herein as containing or near markers associated with AD. In particular embodiments, the markers analyzed in combinations as haplotypes are on chromosome 10q22, 10q23 or 10q24 or are on chromosome 10q23, 10q24 or 10q25. In still further embodiments of these methods, the markers analyzed in combinations as haplotypes for association with AD are located in: (1) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further embodiments of these methods, the markers analyzed in combinations as haplotypes for association with AD are located in a region of chromosome 10 as follows: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In particular embodiments of the haplotype association study-based disease gene localization methods provided herein, markers analyzed in combinations for association with AD are located within a region identified herein as containing or near markers associated with AD.

4. Interaction Analysis

The polymorphic markers described herein may also be used to identify patterns of markers associated with detectable traits resulting from polygenic interactions. The analysis of genetic interaction between unlinked loci (for example alleles on chromosome 10) and APOE4 on chromosome 19) requires individual genotyping using the techniques described herein. The analysis of allelic interaction among a selected set of markers (polymorphisms) with appropriate level of statistical significance can be considered as a haplotype analysis. Interaction analysis involves stratifying the case-control populations with respect to a given haplotype for the first locus and performing a haplotype analysis with the second locus with each subpopulation.

5. Calculation of Linkage Disequilibrium

Linkage disequilibrium is the non-random association of alleles at two or more loci and represents a powerful tool for mapping genes involved in disease traits [see, e.g., Ajioka et al. (1997) Am. J. Human Genet. 60:1439-1447]. Any genetic markers may be used in genetic analysis based on linkage disequilibrium. SNPs, because they are densely spaced in the human genome and can be genotyped in greater numbers than other types of genetic markers (such as micro- or minisatellites), are particularly useful in genetic analysis based on linkage disequilibrium. When not broken up by recombination, “ancestral” haplotypes and linkage disequilibrium between marker alleles at different loci can be tracked not only through pedigrees but also through populations. Direct determination of linkage disequilibrium (as opposed to the obtaining of indirect evidence of linkage disequilibrium as is obtained in association analysis of a marker, or haplotype, and a trait) is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine scale mapping of a disease locus, it is useful to have some knowledge of the patterns of linkage disequilibrium that exist between markers in the studied region. The mapping resolution achieved through the analysis of linkage disequilibrium is much higher than that of linkage studies.

Direct calculation of linkage disequilibrium requires a comparison of two genetic positions and can be used to quantify the extent of linkage disequilibrium in a chromosomal region once a single- or multi-locus disease association has been identified. Haplotypes may be used in linkage disequilibrium calculations.

Several methods can be used to calculate linkage disequilibrium. For example the standardized multiallelic disequilibrium coefficient D′ may be calculated; it has a simple interpretation, its scale is independent of allele frequency and it is applicable to both SNP and microsatellite data [Hedrick (1987) Genetics 117:331-341; Devlin and Risch (1995) Genomics 29:311-322].

6. Cloning and Sequence Analysis of an AD Gene Chromosomal Region

DNA in one or more regions of chromosome 10q, and in particular, 10q22-q26, that have been narrowly defined as a possible AD DNA segment- or AD gene-containing region based on the results of association analyses described herein may be cloned and sequenced. The smaller the candidate AD gene region (e.g., ≦1 Mb or 1 cM and, particularly, ≦500 kb or ≦1 kb) for such analysis, the more efficient the analysis will be. This process may generate additional markers and probes for more detailed mapping of chromosome 10q with respect to the possible location of one or more AD DNA segments or genes.

It may be useful in narrowing in on a candidate AD gene region of chromosome 10, and, in particular, chromosome 10q22-q26, to identify and analyze additional polymorphic markers. Methods of identifying new markers are known in the art. For example, cloned human genomic DNA e.g., cosmids, may be screened for the presence of repetitive DNA stretches (e.g., di, tri, and tetranucleotide repeats). When a sequence is positive for hybridization to a synthetic oligonucleotide, it may be subcloned into a plasmid and sequenced or may be sequenced after PCR amplification of the region. PCR primers may be developed to amplify the repetitive stretch identified, and the markers may be tested for the presence of polymorphism in a panel of control DNAs. In silico procedures using the many human genome and sequence databases may also be used in the identification of additional polymorphic markers.

A procedure for direct identification of microsatellites from yeast artificial chromosomes (YACs) provides several new markers from a target region. This procedure is based on a subtractive hybridization step that permits separation of the target DNA from the vector background. YAC clones containing sequence from the 10q22-26 region have been identified by the CEPH/Genethon consortium (see http://www.cephb.fr/). Markers from YACs that have been mapped to portions of 10q22-q26, and in particular 10q23-q25, that are not well represented by currently available markers can be isolated. The microsatellite identification procedure may be performed by conducting subtractive hybridization using genomic DNA from a target YAC together with an equivalent amount of a control DNA. This procedure separates YAC DNA from that of the yeast vector. Following the subtraction procedure, the subtracted YAC DNA is purified, digested with restriction enzymes and cloned into a plasmid vector. The cloned products of each YAC are screened using a probe for repetitive sequences. Each positive clone is sequenced to identify primers for PCR to genotype AD samples. Alternatively, direct thermal cycle sequencing, based on use of a set of degenerate sequencing primers that anneal directly to the repeat sequence can also be used. By typing these markers in AD families, it is possible to narrow the candidate region, for example, to a size of less than 1-2 cM, thus limiting the segment in which more extensive mapping efforts are applied. Once the candidate region is narrowed, e.g., to a size of less than about 500 to 1000 kb, a contiguous array (contig) of clones with smaller inserts than YACs, P1 clones and/or BACs can be developed. P1 clones are phage clones designed to accommodate inserts of up to 100 kb.

Along with genetic mapping of candidate AD gene chromosomal regions on chromosome 10, a physical map of chromosome 10q, particularly 10q22-q26, including 10q23-q25, may be developed by, for example, assembling contigs of large insert clones that span the region of interest. Low resolution contigs for most of the human genome are available using the YACs and BACs developed by CEPH which may provide a framework to start constructing high resolution contigs. Alternatively, BAC libraries are available or could be developed to cover the region of interest. Contigs may be generated using P1 clones or BACs once the candidate region is narrowed to less than about 1 Mb. Once a region of 500-1000 kb or less is defined, physical mapping and cloning may be accomplished using P1 and/or BAC clones rather than YACs, and P1 or BAC contigs over such a region may be constructed. The P1 or BAC clones may be used to identify additional markers for further positional cloning steps.

A starting point of contig construction is microsatellite sequences and non-polymorphic STSs that derive from YACs that surround a genetically determined AD gene candidate region. The STSs may be used to screen the P1 library. The ends of the P1 clones may be cloned using PCR and used to order the P1 clones relative to each other. Amplification in a new P1 clone may indicate that it overlaps with the previous one. The original set of P1 clones serves as building blocks of the complete contig; each end clone is used to rescreen the library and in this way P1 clones are added to the map. Restriction enzyme digestion and Southern blot hybridization permit ordering of the majority of the P1 clones. From each P1 clone, additional microsatellite markers may be identified which allows further reduction of the candidate region.

From a minimal candidate region defined by genetic and physical mapping analyses, a segment may remain that is sufficiently large to contain multiple different genes. The P1 clones may be used to identify candidate cDNAs of AD genes which may be screened for mutations in DNA from AD subjects. Coding sequences from the surrounding DNA may be identified and screened until a probable candidate cDNA is found. It may also be possible to identify coding sequences through human genome sequence database screening. Candidates may also be identified by scanning databases of partially sequenced cDNAs known as expressed sequence tags (ESTs). The databases may be used to identify cDNAs that map to a minimal AD gene candidate region. The cDNAs may be used as probes to hybridize to a P1 contig, and new microsatellites may be isolated and used to genotype AD subject nucleic acid samples. If maximal linkage disequilibrium is identified in the vicinity of one or two cDNAs, the cDNAs may be the first to be used to screen AD subject DNA for mutations.

Coding sequences may also be identified by exon trapping which targets exons in genomic DNA by identifying the consensus splice sequences that flank exon-intron boundaries. Exons are trapped in the process of cloning DNA, e.g., from P1 clones, into an expression vector. The clones may be transfected into host cells, e.g., COS cells, and reverse transcriptase polymerase chain reaction (RT-PCR) may be performed on total or cytoplasmic RNA isolated from the transfected cells using primers complementary to the splicing vector. Once cDNAs are identified, the most plausible candidates may be screened for sequence variation (mutations or polymorphisms) by direct sequencing, single strand conformation polymorphism (SSCP) or using chemical cleavage assays. Northern blot analysis or reverse transcriptase polymerase chain reaction (RT-PCR) of autopsy tissues from affected and unaffected individuals, RNase protection or chemical cleavage, or any other state-of-the-art technique may also be used in identifying the AD gene or genes on chromosome 10q, particularly in the region of 10q22-q26.

7. Expression of an Identified ORF and Analysis of AD Gene Product

A candidate cDNA, or open reading frame (ORF), may be used to produce a gene product. For example, nucleic acid containing a candidate AD gene coding sequence may be incorporated into an expression vector which typically will include a promoter that is operably linked to the candidate gene coding sequence. Usually, the promoter is a eukaryotic promoter for expression in a mammalian cell. The transcription regulation sequences may include an enhancer recognized by the host cell. The selection of promoters and enhancers depends on the host cell that is used. The vector may also include host-recognized replication systems, amplifiable genes, selectable markers, and other elements.

An expression vector containing a candidate AD gene coding sequence may be introduced into a host cell using any one or more of a variety of methods known in the art. The particular method(s) used may depend on the particular vector and host cell. Suitable methods include, but are not limited to, fusion, conjugation, transfection, transduction, electroporation, injection, calcium phosphate preciptation and many other methods. Suitable host cells include, but are not limited to, bacteria, yeast, filamentous fungi, insect cells and mammalian cells, e.g., mouse, chinese hamster ovary and monkey cells.

Any protein produced upon expression of a candidate AD gene may be isolated using conventional methods of protein biochemistry and purification known in the art. The protein may be analyzed using a variety of methods, including in silico and functional assays. For in silico methods, structural features (e.g., amino acid sequence) of the protein are compared to those known for certain types of proteins (e.g., enzymes) to determine if there are regions of similarity that may be characteristic of such classes of proteins. The information obtained in such studies can provide data used in determining functional aspects of the protein. For functional studies, analysis of the protein may also be conducted in cell-based assays using cells expressing cDNA encoding the protein and/or a gene or cDNA containing genetic alterations, e.g., mutations, correlated with AD. A candidate cDNA, or open reading frame (ORF), may also be expressed in a transgenic, particularly nonhuman, animal. For example, a candidate cDNA may be linked to a promoter, and the resulting construct injected into a zygote, e.g., a mouse zygote. The candidate cDNA or gene may contain one or more genetic alterations, e.g., mutations, correlated with AD. The transgenic animal may also be developed to have one or both alleles of a corresponding endogenous gene inactivated, for example, by using a transgene in which a cloned candidate gene having a positive selection marker-encoding DNA within it such that it no longer encodes the candidate protein. Transgenic animals containing active, mutated and/or inactive candidate genes may be used to study the function and AD-related mechanisms of the protein and in identifying and evaluating drugs for treatment of AD.

H. Methods for Detecting the Presence of a Polymorphism Associated with Alzheimer's Disease

Genetic analysis described herein led to the discovery of genetic association with AD on chromosome 10. The association identifies chromosome 10 as the location of one or more AD DNA segments or genes. Based on this discovery which identifies within the entire genome a particular chromosomal location of an AD DNA segment, methods are provided herein for detecting the presence in a subject of a polymorphism or allele associated with AD. The methods include a specific step of analyzing chromosome 10 of the subject for a polymorphism associated with AD. In particular embodiments of these methods, chromosome 10q is analyzed for a polymorphism associated with AD. In further embodiments of these methods, chromosome 10q22, 10q23, 10q24, 10q25 and/or 10q26 is (are) analyzed for a polymorphism associated with AD. In further embodiments of these methods, a region of chromosome 10 identified herein as containing or near markers associated with AD is analyzed for a polymorphism associated with AD. In particular embodiments, chromosome 10q22, 10q23 and/or 10q24 or chromosome 10q23, 10q24 and/or 10q25 is (are) analyzed for a polymorphism associated with AD. In yet further embodiments of these methods, one or more of the following regions of chromosome 10 is (are) analyzed for a polymorphism associated with AD: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further embodiments, one or more of the following regions of chromosome 10 is (are) analyzed for a polymorphism associated with AD: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In further embodiments of the methods for detecting the presence in a subject of a polymorphism associated with AD, nucleic acid of the subject is analyzed for the presence or absence of marker D10S583. In particular embodiments of the methods for detecting the presence or absence in a subject of a polymorphism associated with AD, chromosome 10 of the subject is analyzed for the presence or absence of a polymorphism which is associated with AD and linked to a DNA segment or gene associated with AD.

Also provided herein are methods for detecting the presence in a subject of a combination or haplotype of polymorphisms associated with AD. In one embodiment, each polymorphism in the combination is associated with AD. In other embodiments, some of the polymorphisms in the combination are associated with AD and some of the polymorphisms are not or none of the polymorphisms is associated with AD. In embodiments in which some or all of the polymorphisms are not individually associated with AD, the combination or haplotype of polymorphisms as a whole may be associated with AD. The methods for detecting the presence in a subject of a combination or haplotype of polymorphisms associated with AD include a specific step of analyzing chromosome 10 of the subject for a combination or haplotype of polymorphisms associated with AD. In particular embodiments of these methods, chromosome 10q is analyzed for a combination or haplotype of polymorphisms associated with AD. In further embodiments of these methods, the polymorphisms in the combination or haplotype associated with AD are located on chromosome 10q22, 10q23, 10q24, 10q25 and/or 10q26. In further embodiments of these methods, a region of chromosome 10 identified herein as containing or near markers associated with AD is analyzed for a combination or haplotype of polymorphisms associated with AD. In particular embodiments, the combination or haplotype of polymorphisms associated with AD are located on chromosome 10q22, 10q23 and/or 10q24 or chromosome 10q23, 10q24 and/or 10q25. In each of these embodiments, the individual polymorphisms contained in the combination or haplotype may be located (1) each in different locations of the identified locations of chromosome 10, (2) all in the same location which is one of the identified locations on chromosome 10, or (3) any number of a variety of arrangements of the individual polymorphisms of the combination within the identified locations of chromosome 10, e.g., one polymorphism in a first location, two polymorphisms in a second location, two polymorphisms in a third location, etc.

In yet further embodiments of these methods, each polymorphism of the combination or haplotype is located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further embodiments, each polymorphism of the combination or haplotype is located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Because of the discovery of chromosome 10 as the location of one or more AD genes, it is possible to determine the presence or absence of one or more polymorphic markers associated with AD in a subject by focussing analysis of a nucleic acid sample of a subject on a particular region or regions of the chromosome identified and described herein. In these methods, the detection of the presence or absence of the polymorphism can be accomplished without having to analyze areas of the subject's genome other than those identified and specified herein. The methods also include, however, optional steps of analyzing other specific areas of the subject's genome which are to known contain markers associated with AD or AD susceptibility or disease alleles.

For example, certain further embodiments of the methods of determining the presence or absence of a polymorphism associated with AD include an additional step of analyzing the subject's nucleic acid sample for the presence or absence of the APOE ε4 allele. Apolipoprotein E (APOE) performs various functions as a protein constituent of plasma lipoproteins, including a role in cholesterol metabolism. The APOE ε4 allele is a well-established susceptibility gene for late-onset AD. The APOE-4 allele is neither necessary or sufficient for AD, but modulates the risk of developing AD [Corder et al. (1993) Science 261:921-923; Corder et al. (1994) Nature Genet. 7:180-184]. The APOE ε4 polymorphism is designated as SNP528 (nucleotide 212240) of Genbank Accession No. AF050154 and is a single nucleotide substitution which results in the replacement of the cysteine at position 112 with an arginine.

Methods of detecting the presence or absence in a subject of a polymorphism associated with AD can be used for several purposes. For example, detection of a polymorphism or allele associated with AD can be used in the identification and isolation of one or more AD DNA segments or genes on chromosome 10 through the genetic and/or physical analysis of specific, relatively small chromosomal regions, particularly 10q22, 10q23, 10q24, 10q25 and/or 10q26, and more particularly, 10q22, 10q23 and/or 10q24 or 10q23, 10q24 and/or 10q25. Detection of a polymorphism or allele associated with AD can also be used in the molecular identification of carriers of an AD gene and a determination of the probability of having the disease or of passing the gene to their offspring. In these methods, the presence of the polymorphism or allele is indicative of carriers of a DNA segment associated with AD. In addition, detection of a polymorphism or allele associated with AD can be used in methods of generating pharmacogenetic profiles which correlate drug response with genotype and thus can also be used in methods of predicting a response of a subject to a drug used to treat AD.

Analysis of chromosome 10 in accordance with the methods of detecting the presence or absence in a subject of a polymorphism associated with AD can be conducted in a variety of ways using analytical methods described herein and genetic analysis methods known to those of skill in the art. Any methods whereby the identity of nucleotides in an nucleic acid sequence may be determined can be used.

Many methods are available for detecting specific alleles at human polymorphic loci. The preferred method for detecting a particular polymorphism, depends on the nature of the polymorphism. Several methods of determining the presence or absence of allelic variants of a human gene are provided below. Methods that are useful are not limited to those described below, but include all available methods.

Generally, nucleic acid detection methods are based in sequence-specific polynucleotides, oligonucleotides, probes and primers. Any method known to those of skill in the art for detecting a specific nucleotide within a nucleic acid sequence or for determining the identity of a specific nucleotide in a nucleic acid sequence is applicable to the methods of determining the presence or absence of an allelic variant on chromosome 10. Such methods include, but are not limited to, techniques utilizing nucleic acid hybridization of sequence-specific probes, nucleic acid sequencing, selective amplification, analysis of restriction enzyme digests of the nucleic acid, cleavage of mismatched heteroduplexes of nucleic acid and probe, alterations of electrophoretic mobility, primer specific extension, oligonucleotide ligation assay and single-stranded conformation polymorphism analysis. In particular, primer extension reactions that specifically terminate by incorporating a dideoxynucleotide are useful for detection. Several such general nucleic acid detection assays are known (see, e.g., U.S. Pat. No. 6,030,778).

Any cell type or tissue may be utilized to obtain nucleic acid samples, e.g., bodily fluid such as blood or saliva, dry samples such as hair or skin.

1. Primer Extension-Based Methods

Several primer extension-based methods for determining the identity of a particular nucleotide in a nucleic acid sequence have been reported (see, e.g., PCT Application Nos. PCT/US96/03651 (WO96/29431), PCT/US97/20444 (WO 98/20166), PCT/US97/20194 (WO 98/20019), PCT/US91/00046 (WO91/13075), and U.S. Pat. Nos. 5,547,835, 5,605,798, 5,622,824, 5,691,141, 5,872,003, 5,851,765, 5,856,092, 5,900,481, 6,043,031, 6,133,436 and 6,197,498.) In general, a primer is prepared that specifically hybridizes adjacent to a polymorphic site in a particular nucleic acid molecule. The primer is then extended in the presence of one or more dideoxynucleotides, typically with at least one of the dideoxynucleotides being the complement of the nucleotide that is polymorphic at the site. The primer and/or the dideoxynucleotides may be labeled to facilitate a determination of primer extension and identity of the extended nucleotide.

A method of genotyping or determining the presence of an allelic variant involves two-dye fluorescence polarization detected single base extension (FP-SBE (12)) on an LJL-Biosystems Criterion Analyst AD (Molecular Devices, Sunnyvale, Calif.). PCR primers are designed to yield products between 200-400 bp in length, and are used at a final concentration of 100-300 nM (Invitrogen Corp., Carlsbad, Calif.) along with Taq polymerase (0.25 U/reaction; Qiagen, Valencia, Calif. and Roche, Indianapolis, Ind.) and dNTPs (2.5 uM/r×n; Amersham-Pharmacia, Piscataway, N.J.). All PCR reactions are performed from ˜10 ng of DNA. General PCR thermo-cycling conditions are as follows: initial denaturation 3 minutes at 94° C., followed by 30-35 cycles of denaturation at 94° C. for 45 seconds, primer-specific annealing temperature (see below) for 45 seconds, and product extension at 72° C. for 1 minute. Final extension at 72° C. for six minutes. PCR products can be visualized on 2% agarose-gels to confirm a single product of the correct size. PCR primers and unincorporated dNTPs can be degraded by adding exonuclease I (Exol, 0.1-0.15 U/reaction; New England Biolabs, Beverly, Mass.) and shrimp alkaline phosphatase (SAP, 1U/reaction; Roche, Indianapolis, Ind.) to the PCR reactions and incubating for 1 hour at 37° C., followed by 15 minutes at 95° C. to inactivate the enzymes. The single base extension step is performed by directly adding SBE primer (100 nM; Invitrogen Corp., Carlsbad, Calif.), Thermosequenase (0.4 U/reaction; Amersham-Pharmacia, Piscataway, N.J.), and the appropriate mixture of R110-ddNTP, TAMRA-ddNTP (3 uM; NEN, Boston, Mass.), and all four unlabeled ddNTPs (22 or 25 uM; Amersham-Pharmacia, Piscataway, N.J.) to the Exol/SAP treated PCR product. Acycloprime-FP SNP detection kits (G/A)(Perkin-Elmer, Boston, Mass.) may also be used for the SBE reaction. Incorporation of the SNP specific fluorescent ddNTP is achieved by subjecting samples to 35 cycles of 94° C. for 15 seconds and 55° C. for 30 seconds. The length of the SBE primers are designed to yield a melting temperature Tₘ of 62-64° C. Fluorescent ddNTP incorporation is detected using the AnalySt™ AD System (Molecular Devices, Sunnyvale, Calif.) and measuring fluorescent polarization for R110 (excitation at 490 nm, emission at 520 nm) and TAMRA (excitation at 550 nm, emission at 580 nm). Genotypes are called manually or automatically using the manufacturer's software (‘Allelecaller vers. 1.0’, Molecular Devices, Sunnyvale, Calif.). In view of the polymorphic regions provided herein, specific PCR primers (5′ to 3′ sequences), annealing temperature, product length, SBE primer sequence, marker location and reference sequence position, can readily be determined by those of skill in the art using well-known methods.

2. Polymorphism-Specific Probe Hybridization

Another detection method is allele specific hybridization using probes overlapping the polymorphic site and having about 5, 10, 15, 20, 25, or 30 nucleotides around the polymorphic region. The probes can contain naturally occurring or modified nucleotides (see U.S. Pat. No. 6,156,501). For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163; Saiki et al. (1989) Proc. Natl. Acad. Sci U.S.A. 86:6230; and Wallace et al. (1979) Nucl. Acids Res. 6:3543). Such allele specific oligonucleotide hybridization techniques may be used for the simultaneous detection of several nucleotide changes in different polymorphic regions. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. In a preferred embodiment, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix, Santa Clara, Calif.). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip includes all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

3. Nucleic Acid Amplification-Based Methods

In other detection methods, it is necessary to first amplify at least a portion of a gene prior to identifying the allelic variant. Amplification can be performed, e.g., by PCR and/or LCR, according to methods known in the art. In one embodiment, genomic DNA of a cell is exposed to two PCR primers and amplification is performed for a number of cycles sufficient to produce the required amount of amplified DNA. In another embodiment, the primers are located between 150 and 350 base pairs apart.

Alternative amplification methods include: self sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

Alternatively, allele specific amplification technology, which depends on selective PCR amplification may be used in conjunction with the alleles provided herein. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). In addition it may be desirable to introduce a restriction site in the region of the mutation to create cleavage-based detection (Gasparini et al. (1992) Mol. Cell Probes 6:1).

4. Nucleic Acid Sequencing-Based Methods

Any of a variety of sequencing reactions known in the art can be used to directly sequence at least a portion of a gene and to detect allelic variants, e.g., mutations, by comparing the sequence of the sample sequence with the corresponding wild-type (control) sequence. Exemplary sequencing reactions include those based on techniques developed by Maxam and Gilbert (1977) Proc. Natl. Acad. Sci. U.S.A. 74:560) or Sanger et al. (1977) Proc. Natl. Acad. Sci 74:5463. It is also contemplated that any of a variety of automated sequencing procedures may be used when performing the subject assays ((1995) Biotechniques 19:448), including sequencing by mass spectrometry (see, for example, U.S. Pat. Nos. 5,547,835, 5,691,141, and International PCT Application No. PCT/US94/00193 (WO 94/16101), entitled “DNA Sequencing by Mass Spectrometry” by H. Koster; U.S. Pat. Nos. 5,547,835, 5,622,824, 5,851,765, 5,872,003, 6,074,823, 6,140,053 and International PCT Application No. PCT/US94/02938 (WO 94/21822), entitled “DNA Sequencing by Mass Spectrometry Via Exonuclease Degradation” by H. Koster, and U.S. Pat. Nos. 5,605,798, 6,043,031, 6,197,498, and International Patent Application No. PCT/US96/03651 (WO 96/29431) entitled “DNA Diagnostics Based on Mass Spectrometry” by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track sequencing or an equivalent, e.g., where only one nucleotide is detected, can be carried out. Other sequencing methods are known (see, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”).

5. Restriction Enzyme Digest Analysis

In some cases, the presence of a specific allele in nucleic acid, particularly DNA, from a subject can be shown by restriction enzyme analysis. For example, a specific nucleotide polymorphism can result in a nucleotide sequence containing a restriction site which is absent from the nucleotide sequence of another allelic variant.

6. Mismatch Cleavage

Protection from cleavage agents, such as, but not limited to, a nuclease, hydroxylamine or osmium tetroxide and with piperidine, can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an allelic variant with a sample nucleic acid, e.g, RNA or DNA, obtained from a tissue sample. The double-stranded duplexes are treated with an agent, which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they differ (see, for example, Cotton et al. (1988) Proc. Natl. Acad Sci U.S.A. 85:4397; Saleeba et al. (1992) Methods Enzymod. 217:286-295). The control or sample nucleic acid is labeled for detection.

7. Electrophoretic Mobility Alterations

In other embodiments, alteration in electrophoretic mobility is used to identify the type of allelic variant of a gene of interest. For example, single-strand conformation polymorphism (SSCP) may be used to detect differences in electrophoretic mobility between mutant and wild type nucleic acids (Orita et al. (1989) Proc. Natl. Acad. Sci. U.S.A. 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method uses heteroduplex analysis to separate double stranded heteroduplex molecules on the basis of changes in electrophoretic mobility (Keen et al. (1991) Trends Genet 7:5).

8. Polyacrylamide Gel Electrophoresis

In yet another embodiment, the identity of an allelic variant of a polymorphic region of an gene is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

### Oligonucleotide Ligation Assay (OLA)

In another embodiment, identification of the allelic variant is carried out using an oligonucleotide ligation assay (OLA), as described, e.g., in U.S. Pat. No. 4,998,617 and in Landegren, U. et al. (1988) Science 241:1077-1080. The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. U.S.A. 87:8923-8927). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucl. Acids Res. 24:3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

9. SNP Detection Methods

Several methods have been developed to facilitate the analysis of single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment, a solution-based method for determining the identity of the nucleotide of a polymorphic site is employed (Cohen, D. et al. (French Patent 2,650,840; PCT Application No. WO91/02087)). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

10. Genetic Bit Analysis

An alternative method, known as Genetic Bit Analysis or GBA™ is described by Goelet, et al. (U.S. Pat. No. 6,004,744, PCT Application No. 92/15712). The method of Goelet, et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO91/02087), the method of Goelet, et al. is preferably a heterogeneous phase assay, in which the primer or the target molecule is immobilized to a solid phase.

11. Other Primer-Guided Nucleotide Incorporation Procedures

Other primer-guided nucleotide incorporation procedures for assaying polymorphic sites in DNA have been described (Komher, J. S. et al. (1989) Nucl. Acids Res. 17:7779-7784; Sokolov, B. P. (1990) Nucl. Acids Res. 18:3671; Syvanen, A. C., et al. (1990) Genomics 8:684-692, Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al. (1993) Amer. J. Hum. Genet. 52:46-59).

For determining the identity of the allelic variant of a polymorphic region located in the coding region of a gene, yet other methods than those described above can be used. For example, identification of an allelic variant which encodes a mutated protein can be performed by using an antibody specifically recognizing the mutant protein in, e.g., immunohistochemistry or immunoprecipitation. Binding assays are known in the art and involve, e.g., obtaining cells from a subject, and performing binding experiments with a labeled lipid, to determine whether binding to the mutated form of the protein differs from binding to the wild-type protein.

12. Molecular Structure Determination

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and single-strand conformation polymorphism.

13. Mass Spectrometric Methods

Nucleic acids can also be analyzed by detection methods and protocols, particularly those that rely on mass spectrometry (see, e.g., U.S. Pat. Nos. 5,605,798, 6,043,031, 6,197,498, and International Patent Application No. WO 96/29431, International PCT Application No. WO 98/20019).

Multiplex methods allow for the simultaneous detection of more than one polymorphic region in a particular gene. This is the preferred method for carrying out haplotype analysis of allelic variants of a gene.

Multiplexing can be achieved by several different methodologies. For example, several mutations can be simultaneously detected on one target sequence by employing corresponding detector (probe) molecules (e.g., oligonucleotides or oligonucleotide mimetics). Variations in additions to those set forth herein will be apparent to the skilled artisan.

A different multiplex detection format is one in which differentiation is accomplished by employing different specific capture sequences which are position-specifically immobilized on a flat surface (e.g., a ‘chip array’).

14. Other Methods

Additional methods of analyzing nucleic acids include amplification-based methods including polymerase chain reaction (PCR), ligase chain reaction (LCR), mini-PCR, rolling circle amplification, autocatalytic methods, such as those using QJ replicase, TAS, 3SR, and any other suitable method known to those of skill in the art.

Other methods for analysis and identification and detection of polymorphisms, include but are not limited to, allele specific probes, Southern analyses, and other such analyses.

I. Methods for Determining the Level of Risk for AD in a Subject

Methods are provided for determining a subject's level of risk for developing or having AD. In these methods, chromosome 10 of the subject is analyzed for the presence or absence of one or more of the following types of polymorphisms or alleles: a polymorphism associated with AD, a polymorphism that is over-represented in cases of a case-control study, a polymorphism associated with unaffected members of a family having members affected with AD and a polymorphism that is under-represented in cases of a case-control study. The presence of a polymorphism or allele associated with AD may be indicative of an increased risk of AD in the subject relative to someone who does not have the polymorphism or allele. The presence of a polymorphism or allele that is over-represented in cases of a case-control study may also be indicative of an increased risk for AD in the subject relative to someone who does not have the polymorphism or allele. The presence of a polymorphism or allele that is associated with AD such that it is associated with unaffected members of a family having members affected with AD may be indicative of a decreased risk for AD in the subject relative to someone who does not have the polymorphism or allele. The presence of a polymorphism or allele that is under-represented in cases of a case-control study may also be indicative of a decreased risk for AD in the subject relative to someone who does not have the polymorphism or allele.

In particular embodiments of these methods for determining a subject's level of risk for developing or having AD, chromosome 10q of the subject is analyzed for the presence or absence of one or more of the polymorphisms or alleles. In further embodiments of these methods, chromosome 10q22, 10q23, 10q24, 10q25 and/or 10q26 is (are) analyzed for the presence or absence of one or more of the polymorphisms or alleles. In yet further embodiments of these methods, a region identified herein as containing or near markers associated with AD is analyzed for the presence or absence of one or more of the polymorphisms or alleles. In particular embodiments of these methods, chromosome 10q22, 10q23 and/or 10q24 is (are) analyzed for the presence or absence of one or more polymorphisms or alleles. In yet a further embodiment, chromosome 10q23, 10q24 and/or 10q25 is (are) analyzed for the presence or absence of one or more polymorphisms or alleles. In further particular embodiments of these methods, one or more of the following regions of chromosome 10 is (are) analyzed for the presence or absence of one or more of the specified polymorphisms or alleles: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In further particular embodiments of these methods, one or more of the following regions of chromosome 10 is (are) analyzed for the presence or absence of one or more of the specified polymorphisms or alleles: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

In particular embodiments of these methods for determining a subject's level of risk for developing or having AD, a region identified herein as containing or near markers associated with AD is analzyed in a subject for the presence or absence of one or more of the specified polymorphisms or alleles.

In particular embodiments of these methods for determining a subject's level of risk for developing or having AD, chromosome 10 of the subject is analyzed for the presence or absence of an allele of marker D10S583, wherein the presence of an allele of D10S583 is indicative of a decreased risk for AD. In particular embodiments of such methods, the allele of D10S583 is about 210 bp and may be, for example, 209 bp or 211 bp.

Thus, the present methods for determining a subject's level of risk for developing or having AD include examination of one or more polymorphic regions of chromosome 10, such as regions of chromosome 10q, and in particular chromosome 10q22, 10q23, 10q24, 10q25 or 10q26. Each allelic variant of a polymorphism may be assayed individually or simultaneously using multiplex assay methods. Any methods described herein or known in the art for determining the identity of nucleotides in a nucleic acid sequence or alterations in a nucleic acid sequence may be used in these methods for the examination of polymorphic regions of chromosome 10. Such methods include, but are not limited to, hybridization using specific oligonucleotides, direct DNA sequencing, restriction enzyme digestion, RNase protection, chemical cleavage and ligase-mediated detection.

Genomic DNA used in the methods may be obtained from body cells, such as, for example, those present in the blood, tissue biopsy, surgical specimen or autopsy material. The DNA may be isolated and used directly for detection of a specific sequence or may be amplified prior to analysis. RNA or cDNA may also be used.

Also provided are methods for determining a subject's level of risk for AD which include a step of analyzing chromosome 10 of the subject for the presence or absence of a combination or haplotype of polymorphisms or alleles as follows: a combination or haplotype of polymorphisms associated with AD, a combination or haplotype of polymorphisms that is over-represented in cases of a case-control study, a combination or haplotype of polymorphisms associated with unaffected members of a family having members affected with AD and a combination or haplotype of polymorphisms that is under-represented in cases of a case-control study. The presence of a combination or haplotype of polymorphisms or alleles associated with AD may be indicative of an increased risk of AD in the subject relative to someone who does not have the combination or haplotype. The presence of a combination or haplotype of polymorphisms or alleles that is over-represented in cases of a case-control study may also be indicative of an increased risk for AD in the subject relative to someone who does not have the combination or haplotype. The presence of a combination or haplotype of polymorphisms or alleles that is associated with AD such that it is associated with unaffected members of a family having members affected with AD may be indicative of a decreased risk for AD in the subject relative to someone who does not have the combination or haplotype. The presence of a combination or haplotype of polymorphisms or alleles that is under-represented in cases of a case-control study may also be indicative of a decreased risk for AD in the subject relative to someone who does not have the combination or haplotype. In these methods, each polymorphism of the combination or haplotype may be located in any one of the regions of chromosome 10 explicitly set forth above with respect to the methods for determining a subject's level of risk for AD which include a step of analyzing chromosome 10 of the subject for the presence or absence of one or more polymorphisms associated with AD, that is over-represented in cases of a case-control study, associated with unaffected members of a family having members affected with AD or that is under-represented in cases of a case-control study.

J. Methods for Indicating a Predisposition to or Occurrence of Alzheimer's Disease

Methods are provided for indicating a predisposition to or the occurrence of Alzheimer's disease in a subject, which include a step of detecting in nucleic acid obtained from the subject the presence or absence of a polymorphism or allele on chromosome 10 associated with AD. In these methods, the presence of the polymorphism is indicative of a predisposition to or the occurrence of AD.

In particular embodiments of the methods for indicating a predisposition to or the occurrence of Alzheimer's disease in a subject, the polymorphism or allele is on chromosome 10q. In further embodiments of these methods, the polymorphism is on chromosome 10q22, 10q23, 10q24, 10q25 or 10q26. In further embodiments of these methods, the polymorphism is in a region of chromosome 10 identified herein as containing or near markers associated with AD is analyzed for a polymorphism associated with AD. In particular embodiments, the polymorphism is on chromosome 10q22, 10q23 or 10q24 or on chromosome 10q23, 10q24 or 10q25. In yet further embodiments of these methods, the polymorphism or allele is located on one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, the polymorphism or allele is located on one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Thus, the present methods for determining a predisposition to or occurrence of Alzheimer's disease include examination of one or more polymorphic regions of chromosome 10, in particular chromosome 10q, and in particular chromosome 10q22, 10q23, 10q24, 10q25 or 10q26. Each allelic variant of a polymorphism may be assayed individually or simultaneously using multiplex assay methods. Any methods described herein or known in the art for determining the identity of nucleotides in a nucleic acid sequence or alterations in a nucleic acid sequence may be used in these methods for the examination of polymorphic regions of chromosome 10. Such methods include, but are not limited to, hybridization using specific oligonucleotides, direct DNA sequencing, restriction enzyme digestion, RNase protection, chemical cleavage and ligase-mediated detection.

Genomic DNA used in the methods may be obtained from body cells, such as, for example, those present in the blood, tissue biopsy, surgical specimen or autopsy material. The DNA may be isolated and used directly for detection of a specific sequence or may be amplified prior to analysis. RNA or cDNA may also be used.

Also provided are methods for determining a predisposition to or occurrence of Alzheimer's disease in a subject which include a step of detecting in nucleic acid obtained from the subject the presence or absence of a combination or haplotype of polymorphisms or alleles on chromosome 10 associated with AD. In these methods, the presence of the combination or haplotype of polymorphisms is indicative of a predisposition to or the occurrence of AD. In particular embodiments of the methods for indicating a predisposition to or the occurrence of Alzheimer's disease in a subject, the combination or haplotype of polymorphisms or alleles is on chromosome 10q. In further embodiments of these methods, each of the polymorphisms of the combination or haplotype is on chromosome 10q22, 10q23, 10q24, 10q25 or 10q26. In further embodiments of these methods, each of the polymorphisms of the combination or haplotype is in a region of chromosome 10 identified herein as containing or near markers associated with AD. In particular embodiments, each of the polymorphisms of the combination or haplotype is on chromosome 10q22, 10q23 or 10q24 or on chromosome 10q23, 10q24 or 10q25. In yet further embodiments of these methods, each of the polymorphisms or alleles of the combination or haplotype is located on one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 21 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, each of the polymorphisms or alleles of the combination or haplotype is located on one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

K. Methods for Predicting Response to Treatment for Alzheimer's Disease and for Treating Alzheimer's Disease

There are a number of approved prescription therapies available for the treatment of AD, including the cholinesterase inhbibitors tacrine, donepezil and rivastigmine. The presence or absence of one or more polymorphisms or alleles on chromosome 10 which are associated with AD may correlate with a subject's response to a specific therapeutic drug. In determining such a pharmacogenetic profile, one or more alleles or polymorphic markers of chromosome 10 are correlated with drug response by obtaining genotype and/or haplotype data from various groups of patients, in particular, patients diagnosed phenotypically with AD, to whom the drug has been administered. The genotype and/or haplotype of the subject can then allow a clinician to take a more individualized approach to preventing the onset, reducing the progression or alleviating symptoms of AD by tailoring the therapy to increase the chance of a favorable effect.

Other therapeutic agents that can be profiled include, but are not limited to, ALCAR, Alpha-tocopherol (Vitamin E), Ampalex, AN-1792 (AIP-001), Cerebrolysin, Daposone, Donepezil (Aricept), ENA-713 (Exelon), Estrogen replacement therapy, Galanthamine (Reminyl), Ginkgo Biloba extract, Huperzine A, Ibuprofen, Lipitor, Naproxen, Nefiracetam, Neotrofin, Memantine, Phenserine, Rofecoxib, Selegiline (Eldepryl), Tacrine (Cognex), Xanomeline (skin patch), Resperidone (Risperidol™), Neuroleptics, Benzodiazepenes, Valproate, Serotonin reuptake inhibitors (SRIs), Beta and Gamma Secretase Inhibitors, CX-516 (Ampalex), Statins and AF-102B (Evoxac).

Other therapeutic agents include those that are neuroprotective. Drugs with anti-oxidative properties, e.g., flupirtine, N-acetylcysteine, idebenone, melatonin, and also novel dopamine agonists (ropinirole and pramipexole) have been shown to protect neuronal cells from apoptosis and thus have been suggested for treating neurodegenerative disorders like AD or PD. Also, free radical scavengers, calcium channel blockers and modulators of certain signal transduction pathways that might protect neurons from downstream effects of the accumulation of A-Beta intracellularly and/or extracellularly. Also, other agents like non-steroidal anti-inflammatory drugs (NSAIDs) partly inhibit cyclooxygenase (COX) expression, as well as having a positive influence on the clinical expression of AD. Distinct cytokines, growth factors and related drug candidates, e.g., nerve growth factor (NGF), or members of the transforming growth factor-beta (TGF-beta) superfamily, like growth and differentiation factor 5 (GDF-5), are shown to protect tyrosine hydroxylase or dopaminergic neurones from apoptosis. CRIB (cellular replacement by immunoisolatory biocapsule) is gene therapeutical approach for human NGF secretion, which has been shown to protect cholinergic neurones from cell death when implanted in the brain ((2000) Expert Opin Investig Drugs 9(4):747-64).

Provided herein are methods for predicting a response of a subject to a drug used to treat Alzheimer's disease by detecting the presence or absence of a polymorphism or allele on chromosome 10 which is associated with AD. A collection or combination or haplotype of polymorphic regions that individually represent allelic variants that are associated with AD or that as a group on the whole are associated with AD may be more informative than a single allelic variant for indicating whether an individual will positively respond to a given drug for AD. Each allelic variant may be assayed individually or simultaneously using multiplex assay methods, such as multiplex primer extension assays or microarrays comprising probes for specific alleles.

Accordingly, provided herein are methods for predicting a response of a subject to a drug used to treat Alzheimer's disease which include a step of detecting the presence or absence of at least one polymorphism or allele on chromosome 10 that is associated with AD, wherein the presence of the polymorphism or allele is indicative of an increased or decreased likelihood that the drug treatment for AD will be effective. In particular embodiments of either of these methods, the polymorphism is located on chromosome 10q. In further embodiments, the polymorphism is located on chromosome 10q22, 10q23, 10q24, 10q25 or 10q26. In yet further embodiments of these methods, the polymorphism is in a region of chromosome 10 identified herein as containing or near markers associated with AD. In particular embodiments, the polymorphism is on chromosome 10q22, 10q23 or 10q24 or on chromosome 10q23, 10q24 or 10q25. In yet further embodiments of these methods, the polymorphism is located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, the polymorphism or allele is located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Also provided are methods for predicting a response of a subject to a drug used to treat Alzheimer's disease by detecting the presence or absence of a combination or haplotype of polymorphisms or alleles on chromosome 10 which is associated with AD, wherein the presence of the combination or haplotype of polymorphisms or alleles is indicative of an increased or decreased likelihood that the drug treatment for AD will be effective. In particular embodiments of either of these methods, the combination or haplotype of polymorphisms is located on chromosome 10q. In further embodiments, each of the polymorphisms of the combination or haplotype is located on chromosome 10q22, 10q23, 10q24, 10q25 or 10q26. In yet further embodiments of these methods, each of the polymorphisms of the combination or haplotype is in a region of chromosome 10 identified herein as containing or near markers associated with AD. In particular embodiments, each of the polymorphisms of the combination or haplotype is on chromosome 10q22, 10q23 or 10q24 or on chromosome 10q23, 10q24 or 10q25. In yet further embodiments of these methods, the each of the polymorphisms of the combination or haplotype is located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, each of the polymorphisms or alleles of the combination or haplotype is located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Further provided are methods of treating a subject manifesting an Alzheimer's disease phenotype. Certain ambiguous phenotypes, e.g., dementia, mainfested in AD also occur in connection with other diseases and conditions which may be treated using drugs and other treatments that are different from drugs and methods used to treat AD. Genotyping of chromosome 10 markers described herein, and optionally other AD-associated markers, in subjects manifesting such an AD phenotype(s) permits confirmation of AD phenotypic diagnoses and assists in distinguishing between AD and other possible diseases or disorders. Once an individual is genotyped as having or being predisposed to AD, he or she may be treated with any known methods effective in treating AD.

Accordingly, methods of treating a subject manifesting an Alzheimer's disease phenotype provided herein include steps of (a) detecting in nucleic acid obtained from the subject the presence of a polymorphism or allele on chromosome 10 associated with AD, which is indicative of the occurrence of AD, and (b) selecting a treatment plan that is effective for treatment of Alzheimer's disease. In particular embodiments of these methods, the polymorphism or allele is located on chromosome 10q. In further embodiments of these methods, the polymorphism or allele is located on chromosome 10q22, 10q23, 10q24, 10q25 or 10q26. In further embodiments of these methods, the polymorphism or allele is in a region of chromosome 10 identified herein as containing or near markers associated with AD is analyzed for a polymorphism associated with AD. In particular embodiments, the polymorphism is located on chromosome 10q22, 10q23 or 10q24 or on chromosome 10q23, 10q24 or 10q25. In yet further embodiments of these methods, the polymorphism or allele is located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, the polymorphism or allele is located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

Also provided are methods of treating a subject manifesting an Alzheimer's disease phenotype provided herein include steps of (a) detecting in nucleic acid obtained from the subject the presence of a combination or haplotype of polymorphisms or alleles on chromosome 10 associated with AD, which is indicative of the occurrence of AD, and (b) selecting a treatment plan that is effective for treatment of Alzheimer's disease. In particular embodiments of these methods, the combination or haplotype of polymorphisms or alleles is located on chromosome 10q. In further embodiments of these methods, each of the polymorphisms or alleles of the combination or haplotype is located on chromosome 10q22, 10q23, 10q24, 10q25 or 10q26. In further embodiments of these methods, each of the polymorphisms or alleles of the combination or haplotype is in a region of chromosome 10 identified herein as containing or near markers associated with AD is analyzed for a polymorphism associated with AD. In particular embodiments, each of the polymorphisms of the combination or haplotype is located on chromosome 10q22, 10q23 or 10q24 or on chromosome 10q23, 10q24 or 10q25. In yet further embodiments of these methods, each of the polymorphisms or alleles of the combination or haplotype is located in one or more of the following regions of chromosome 10: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of these methods, each of the polymorphisms or alleles of the combination or haplotype is located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

L. Nucleic Acid Compositions, Kits, Articles of Manufacture and Combinations

Provided herein are combinations of two or more or three or more oligonucleotides which hybridize to, or adjacent to, a DNA segment within chromosome 10 that is associated with AD or hybridize to DNA flanking the DNA segment. In particular embodiments of the combinations, the oligonucleotides hybridize to, or adjacent to, a DNA segment in chromosome 10q or hybridize to DNA flanking the DNA segment. In further embodiments of the combinations, the oligonucleotides hybridize to, or adjacent to, a DNA segment in chromosome 10q22, 10q23, 10q24, 10q25 and/or 10q26 or hybridize to DNA flanking the DNA segment. In particular embodiments of the combinations, the oligonucleotides hybridize to, or adjacent to, or to DNA flanking a DNA segment in a region of chromosome 10 identified herein as containing or near markers associated with AD. In further embodiments of the combinations, the oligonucleotides hybridize to, or adjacent to, or to DNA flanking a DNA segment on chromosome 10q22, 10q23 and/or 10q24 or on chromosome 10q23, 10q24 and/or 10q25. In yet further embodiments of the combinations, the oligonucleotides hybridize to, or adjacent to, or to DNA flanking a DNA segment in chromosome 10 located in one or more of the following regions: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of the combinations, the oligonucleotides hybridize to, or adjacent to, or to DNA flanking a DNA segment in chromosome 10 located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

The oligonucleotides in particular combinations may contain 15 or more contiguous nucleotides of the following nucleotide sequences: SEQ ID NOS. 3-14. For example, a combination provided herein may contain two or more of any of the following pairs of oligonucleotides (1) 15 or more contiguous nucleotides of SEQ ID NO. 3 and 15 or more contiguous nucleotides of SEQ ID NO. 4, (2) 15 or more contiguous nucleotides of SEQ ID NO. 5 and 15 or more contiguous nucleotides of SEQ ID NO. 6, (3) 15 or more contiguous nucleotides of SEQ ID NO. 7 and 15 or more contiguous nucleotides of SEQ ID NO. 8, (4) 15 or more contiguous nucleotides of SEQ ID NO. 9 and 15 or more contiguous nucleotides of SEQ ID NO. 10, (5) 15 or more contiguous nucleotides of SEQ ID NO. 11 and 15 or more contiguous nucleotides of SEQ ID NO. 12, and (6) 15 or more contiguous nucleotides of SEQ ID NO. 13 and 15 or more contiguous nucleotides of SEQ ID NO. 14.

In particular embodiments of the combinations provided herein, the oligonucleotides hybridize to, or adjacent to, or to DNA flanking two or more markers, one of which is D10S583. For example, a combination provided herein may contain two or more pairs of oligonucleotides, one pair of which includes 15 or more contiguous nucleotides of SEQ ID NO. 5 and 15 or more contiguous nucleotides of SEQ ID NO. 6.

In particular combinations that have only two oligonucleotides, the two oligonucleotides hybridize to, or adjacent to, or to DNA flanking different DNA segments in chromosome 10, as described herein. That is, the two oligonucleotides may be used to detect two different DNA segments and are not a pair of oligonucleotides used to detect a single DNA fragment, such as, for example, a pair of PCR primers that amplify a single DNA segment. In particular combinations that have only three oligonucleotides, at least two of the oligonucleotides hybridize to, or adjacent to, or to DNA flanking different DNA segments in chromosome 10, as described herein.

Also provided herein are kits and articles of manufacture for use in the practice of methods described herein. The kits and articles of manufacture include one or more containers (e.g., a tube or vial) which contain one or more or two or more oligonucleotides which hybridize to, or adjacent to, a DNA segment within chromosome 10 that is associated with AD or hybridize to DNA flanking the DNA segment. Thus, included within the kits and articles of manufacture are packaged forms of the combinations provided herein. In particular embodiments of the kits and articles of manufacture, the oligonucleotides hybridize to, or adjacent to, a DNA segment in chromosome 10q. In further embodiments of the kits and articles of manufacture, the oligonucleotides hybridize to, or adjacent to, a DNA segment in chromosome 10q22, 10q23, 10q24, 10q25 and/or 10q26 or hybridize to DNA flanking the DNA segment. In particular embodiments of the kits and articles of manufacture, the oligonucleotides hybridize to, or adjacent to, or to DNA flanking a DNA segment in a region of chromosome 10 identified herein as containing or near markers associated with AD. In further embodiments, oligonucleotides hybridize to, or adjacent to, or to DNA flanking a DNA segment on chromosome 10q22, 10q23 and/or 10q24 or on chromosome 10q23, 10q24 and/or 10q25. In yet further embodiments of the kits and articles of manufacture, the oligonucleotides hybridize to, or adjacent to, or to DNA flanking a DNA segment in chromosome 10 located in one or more of the following regions: (1) the region extending about 50 cM, or about 45 cM, or about 33 cM, or about 30 cM, or about 25 cM, or about 21 cM, or about 20 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 60 cM, or about 55 cM, or about 50 cM, or about 45 cM, or about 40 cM, or about 35 cM, or about 30 cM, or about 28 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583, (3) the region extending from and including marker D10S583 to the terminus of the q arm of chromosome 10, (4) the region extending from and including marker D10S583 to the centromere of chromosome 10, (5) the region extending about 62 Mb, about 55 Mb, or about 50 Mb, or about 32 Mb, or about 25 Mb, or about 20 Mb, or about Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583, (6) the region extending about 45 Mb, or about 40 Mb, or about 35 Mb, or about 30 Mb, or about 25 Mb, or about Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583, (7) the region between D10S564 and D10S583, inclusive, (8) the region between D10S583 and D10S1710, inclusive, (9) the region between D10S583 and D10S566, inclusive, (10) the region between D10S583 and D10S1671, inclusive and (11) the region between D10S583 and D10S1741, inclusive.

In yet further embodiments of the kits and articles of manufacture, the oligonucleotides hybridize to, or adjacent to, or to DNA flanking a DNA segment in chromosome 10 located in one or more of the following regions of chromosome 10: (1) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583, (2) the region extending about 5 cM, or about 2.5 cM, or about 1 cM, or about 0.5 cM or about 0.1 cM distal (telomeric) from and including marker D10S583, (3) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583, and (4) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

The oligonucleotides in particular kits and articles of manufacture may contain 15 or more contiguous nucleotides of the following nucleotide sequences: SEQ ID NOS. 1-12. For example, a kit or article of manufacture provided herein may contain one or more or two or more of the following oligonucleotide pairs: (1) 15 or more contiguous nucleotides of SEQ ID NO. 3 and 15 or more contiguous nucleotides of SEQ ID NO. 4, (2) 15 or more contiguous nucleotides of SEQ ID NO. 5 and 15 or more contiguous nucleotides of SEQ ID NO. 6, (3) 15 or more contiguous nucleotides of SEQ ID NO. 7 and 15 or more contiguous nucleotides of SEQ ID NO. 8, (4) 15 or more contiguous nucleotides of SEQ ID NO. 9 and 15 or more contiguous nucleotides of SEQ ID NO. 10, (5) 15 or more contiguous nucleotides of SEQ ID NO. 11 and 15 or more contiguous nucleotides of SEQ ID NO. 12, and (6) 15 or more contiguous nucleotides of SEQ ID NO. 13 and 15 or more contiguous nucleotides of SEQ ID NO. 14.

In particular embodiments of the kits and articles of manufacture provided herein, the oligonucleotides hybridize to, or adjacent to, or to DNA flanking one or more or two or more of markers, one of which is D10S583. For example, a kit or article of manufacture provided herein may contain one or more or two or more oligonucleotide pairs, one pair of which includes 15 or more contiguous nucleotides of SEQ ID NO. 5 and 15 or more contiguous nucleotides of SEQ ID NO. 6.

The kits and articles of manufacture provided herein may optionally include other elements or reagents in addition to the one or more or two or more oligonucleotides which hybridize to, or adjacent to, or to DNA flanking a DNA segment within chromosome 10, and particularly within specific regions of chromosome 10 described herein, that is associated with AD. For example, the kits and articles of manufacture may also include one or more oligonucleotides that hybridize to, or adjacent to, or to DNA flanking a DNA segment located on a chromosome other than chromosome 10 that is associated with AD or linked to a gene associated with AD. In a particular embodiment, such oligonucleotides include those that hybridize to, or adjacent to, or to DNA flanking the APOE gene. Another element which additionally may be included in the kits and articles of manufacture is labelling or instructions indicating the suitability of the kits for detecting a polymorphism associated with AD, determining a predisposition to or occurrence of AD, predicting a response to a treatment for AD and/or treating AD.

The combinations, kits and articles of manufacture provided herein may be used in a variety of methods. For example, combinations, kits and articles of manufacture may be used to determine the presence or absence in nucleic acid obtained from a subject of a polymorphism associated with AD. In a particular embodiment of a kit or article of manufacture that may be used for this purpose, the kit or article of manufacture may include labeling or instructions describing procedures for using the oligonucleotide(s) contained in the kit or article of manufacture in the detection of one or more or two or more polymorphisms on chromosome 10 associated with AD.

Similarly, the combinations, kits and articles of manufacture may be used, for example, to determine a level of risk for AD, to determine a predisposition to or occurrence of AD in a subject, to predict a subject's response to a treatment for AD and/or to treat AD in a subject. In particular embodiments of a kit or article of manufacture that may be used for these purposes, the kit or article of manufacture may include labeling or instructions describing procedures for using the oligonucleotide(s) contained in the kit or article of manufacture in methods of detecting the presence or absence of one or more or two or more polymorphisms on chormosome 10 associated with AD, wherein the procedures provide results on which to base a determination of the a subject's level of risk for AD, a predisposition to or occurrence of AD in a subject, a prediction of a subject's response to a treatment for AD and/or a treatment of AD in a subject.

For any of these or other uses, the kits or articles of manufacture may contain one or more or two or more pairs of oligonucleotides and instructions or labelling describing use of the oligonucleotide pairs to amplify one or more or two or more regions of chromosome 10 at which polymorphisms associated with AD. The labelling or instructions may further specify possible results of the amplification reaction(s) and interpretation thereof as to whether the results are indicative of the presence or absence of a polymorphism associated with AD or linked to a DNA segment associated with AD. In another example, the kit or article of manufacture may contain one or more or two or more oligonucleotides and instructions or labelling describing use of the oligonucleotide(s) in hybridization, sequencing and/or primer extension-based methods of detecting the presence of a polymorphism associated with AD. The labelling or instructions may further specify possible results of the methods and interpretation thereof as to whether the results are indicative of the presence or absence of a polymorphism associated with AD.

The following examples are included for illustrative purposes only and are not intended to limit the scope of the invention.

### EXAMPLE 1

Genotyping of Chromosome 10 Markers in DNA from AD Family Members

Microsatellite markers on human chromosome 10 were analyzed for genetic linkage to AD. The analysis was conducted by genotyping genomic DNA samples from AD family members with respect to seven microsatellite markers and performing parametric and nonparametric analyses of genotyping data.

Genomic DNA Samples

The genomic DNA utilized in the linkage analyses was from the full National Institute of Mental Health (NIMH) Genetics Initiative sample of AD family DNA [Blacker et al. (1997) Neurology 48:139-147]. Through the NIMH Genetics Initiative, a national resource of clinical data and biomaterials (DNA samples) collected from individuals with AD has been established. AD pedigrees have been ascertained by three extramural sites (Massachusetts General Hospital/Harvard Medical School, University of Alabama and Johns Hopkins University) and data collection has been coordinated among the three sites by using a common protocol that includes uniform assessments and medical, neurologic and psychiatric histories.

In generating the NIMH sample, subjects were collected following a standardized protocol applying NINCDS/ADRDA (National Institute of Neurological and Communicative Disorders and Stroke/Alzheimer's Disease and Related Disorders Association) criteria for the diagnosis of AD [Blacker et al. (1997) Neurology 48:139; McKhann et al. (1984) Neurology 34:939-944]. The diagnostic process in the NIMH AD Genetics Initiative includes a systematic and comprehensive examination of all available information from autopsy records, family history, medical records, and patient and/or informant interviews. Definite AD according to age-adjusted Khachaturian criteria is diagnosed on autopsy. Operational criteria for the clinical diagnosis of probable or possible AD following NINCDS-ADRDA Work Group guidelines have been developed and are implemented by the three sites. Case summaries for all subjects with a clinical diagnosis of probable or possible AD are reviewed by the site principal investigators and a procedure has been implemented to establish a consensus diagnosis. Subjects are followed longitudinally to track changes in diagnoses and to compare diagnoses by autopsy.

Only families in which all sample affecteds had onset ages ≧50 years were included (n=435 families; n=1426 subjects, mean age of onset=72.5±7.7 years, range 50-97 years). The original sample included a total of 1500 subjects from 449 families with two or more affected subjects per family. Families in which any sampled individual had an onset age less than 50 years (n=14 families and 74 individuals) were excluded, yielding 1426 individuals from 435 families for this analysis, including 993 affected individuals, 429 unaffected, and 4 with phenotype unknown. Over the 10 years that the NIMH sample has been followed, a clinical diagnosis of AD has been confirmed at autopsy in 94% of the cases. All DNA samples are stored in a centralized cell repository at Rutgers University, New Brunswick, N.J.

Genotyping of Genomic DNA

The DNA samples were genotyped with respect to seven microsatellite markers located on chromosome 10 and APOE. In selecting the markers for genotyping on chromosome 10, the following genetic maps were considered: NCBI (http://www.ncbi.nim.nih.gov/), LDB (http://cedar.genetics.soton.ac.uk/public_html), Marshfield Center for Medical Genetics (http://research.marshfieldclinic.org/genetics/) and GDB (http://www.gdb.org/gdb/). Six of the seven microsatellite markers on chromosome 10 are located on 10q23-q25 and the remaining marker (D10S1225) is located on 10q21.3 [according to the UCSC Golden Path Genome Browser (http://genome.ucsc.edu/index.html). The seven markers, and their positions on the sex-averaged Marshfield map, are: D10S1225 (80.8 cM), D10S564 (112.6 cM), D10S583 (115.3 cM), D10S1710 (124.3 cM), D10S566 (127.1 cM), D10S1671 (127.1 cM) and D10S1741 (128.2 cM). TABLE 2 provides additional information concerning each locus marker.

TABLE 2

| | | | | GenBank | |
|---|---|---|---|---|---|
| | | Location | | Accession | Hetero- |
| Locus | Marker | (cM)^{a} | Type^{b} | No. | zygosity |
| D10S1225 | ATA24F10 | 80.77 | TRI | G08772 | 0.78 |
| D10S564 | AFM029xh12 | 112.58 | DI | Z23295 | 0.62 |
| D10S583 | AFM289zh5 | 115.27 | DI | Z24088 | 0.87 |
| D10S1710 | AFMb055zb9 | 124.27 | DI | Z53234 | 0.60 |
| D10S566 | AFM154xh2 | 127.11 | DI | Z23404 | 0.65 |
| D10S1671 | AFMa222zc5 | 127.11 | DI | Z52570 | 0.72 |
| D10S1741 | AFMc005xh5 | 128.19 | DI | Z53940 | 0.58 |

^{a}Kosambi cM
^{b}Marker types: DI = dinucleotide repeat; TRI = trinucleotide repeat.

Marker genotypes were available for, on average, 82% of the sampled subjects. Genotyping of the microsatellite markers was conducted using nucleic acid amplification procedures essentially as follows. Genomic DNA, ˜30 ng, in a 10 μl reaction volume, was amplified in a polymerase chain reaction (PCR). The reaction volume contained 3 pmol of each primer (primer oligonucleotides were synthesized by Life Technologies, Rockville, Md.), 5 μl or Taq PCR Master Mix (Qiagen, Inc.) and 0.1 μl of [α-33P]dATP (1 μCi). After amplification, the products were denatured for 3 min at 94° C. and then separated by polyacrylamide gel electrophoresis (6%, National Diagnostics, Atlanta, Ga.) for 1-3 hours at 40-60 Watts and exposed to film for 24-48 hours. PCR conditions were essentially those specified by GDB. The primers for amplification of each locus marker are shown in TABLE 3.

TABLE 3

| | | | | | |
|---|---|---|---|---|---|
| | | | | Size | SEQ |
| | | Forward | Reverse | Range | ID |
| Locus | Marker | Primer | Primer | (bp)^{a} | NO^{b} |

| | | | | | |
|---|---|---|---|---|---|
| D10S1225 | ATA24F10 | TCCTTTGT | GTTACATAG | 173- | 1, 2 |
| | | CAGTCATGC | GAAGCCCG | 208 | |
| | | TCA | | | |
| D10S564 | AFM029xh12 | TGGGAATGT | AGCTCTAACAT | 252- | 3, 4 |
| | | GTCTTTATC | AGAGGCCAGAT | 262 | |
| | | CA | | | |
| D10S583 | AFM289ZH5 | TCTGACCAA | AGAGACTCCAG | 201- | 5, 6 |
| | | AATACCAAA | ATGTTTGATGA | 219 | |
| | | AGAAC | | | |
| D10S1710 | AFMb055zb9 | GTCTCAGTC | TTCATCTTACTC | 194- | 7, 8 |
| | | TCCAGGTGA | AAAGTGGGGC | 206 | |
| | | CAATG | | | |
| D10S566 | AFM154xh2 | CTGAGGTAG | AGCTGTGAAAA | 200- | 9, |
| | | GGGGATGGC | ATCACATATTGC | 212 | 10 |
| | | TT | TA | | |
| D10S1671 | AFMa222zc5 | ATGCAATGA | TGATGATGCTC | 92- | 11, |
| | | GTGCTCCCT | AAATGATGG | 110 | 12 |
| D10S1741 | AFMc005xh5 | GAGTAGTGT | AGATGTTCAGTT | 210- | 13, |
| | | CATGGCTCC | CCTTGGCT | 254 | 14 |
| | | CT | | | |

^{a}Size range for the amplified fragments for each marker.
^{b}SEQ ID NO for the forward and reverse primers, respectively.

APOE was genotyped as follows [see also methods described in Blacker et al. (1997) Neurology 48:140]. Genomic DNA was amplified by polymerase chain reaction using the following primers:

| | |
|---|---|
| 5′-TCCAAGGAGCTGCAGGCGGCGCA3′ | SEQ ID NO: 15 |
| 5′-ACAGAATTCGCCCCGGCCTGGTACACTGCCA-3′ | SEQ ID NO: 16 |

For each amplification, 20 ng of human genomic DNA, 1 ng of each primer, 200 μM of dCTP, dTTP and dGTP, 25 μM dATP, 20 pCi (alpha-³³P) dATP, 10% DMSO, 0.1 μl of 100×BSA, 1.6 units Taq DNA polymerase (5 units/μl; Fisher Biotech, Agawan, Mass.), and 1×reacton buffer (supplied by vendor with 15 mM MgCl₂) were combined in a final volume of 10 μl. Reactions were conducted in V-well plates in a PTC-100 Programmable Thermal Controller under the following conditions: 5 min at 94° C., 34 cycles of 30 sec at 94° C., 30 sec at 69° C., 1.5 min at 70° C., followed by a final extension step at 70° C. for 10 min. APOE isotypes were then determined by cleaving with the restriction enzyme Hhal (5 units) added directly to each well and incubated at 37° C. for 6 hours. Ten μl of 2×stop dye were added to each well and 5 μl of this mix was then loaded into each well of a 6% nondenaturing polyacrylamide gel. Following electrophoresis at 45 mA for 1.5 hr, the gel was transferred to Whatman 3M chromatography paper, dried by vacuum and exposed to Kodak XAR-5 film. Autoradiography was carried out for 1 to 16 hrs at −70° C. The resulting genotypes on each autoradiograph were read independently by two different observers and scoring of the alleles was determined, as presented in Hixon and Vernier [(1990) J. Lipid Res. 31:545].

Other Genotyping Methods

A variety of methods may be used in genotyping nucleic acids for particular markers. Any source of nucleic acids, in purified or non-purified form, can be utilized as the starting nucleic acid. DNA or RNA may be extracted from cells, tissues or body fluids.

Many genotyping methods, although not all, involve amplification of the nucleic acid region carrying the marker of interest. Such methods specifically increase the concentration or total number of sequences that span the marker. Some amplification methods allow for detection of a polymorphism and simultaneous amplification of a target sequence. Diagnostic assays may also rely on amplification of DNA segments carrying a marker. While the amplification of the target segment is often employed in genotyping methods, there are ultrasensitive detection methods known in the art that do not require amplification.

Amplification of nucleic acids may be achieved by any method known in the art. Oligonucleotides appropriate for use as primers in nucleic acid amplification of chromosome 10 markers include those provided herein as well as any others that serve to specifically amplify segments of chromosome 10 containing a marker. The spacing of primers determines the length of the segment to be amplified. Amplified segments carrying markers can range in size from about 25 bp to kilobases, and may typically be on the order of hundreds of bp. Amplification primers may be labeled or immobilized on a solid support.

Methods known to those skilled in the art which can be used to detect markers in genotyping include methods such as conventional dot blot analyses, single strand conformational polymorphism analysis [Orita et al. (1989) Proc. Natl. Acad. Sci. U.S.A. 86:2776-2770], denaturing gradient gel electrophoresis, heteroduplex analysis, mismatch cleavage detection, and other techniques [see, e.g., Sheffield et al. (1991) Proc. Natl. Acad. Sci. U.S.A. 49:699-706; White et al. (1992) Genomics 12:301-306; Grompe et al. (1989) Proc. Natl. Acad. Sci. U.S.A. 86:5855-5892; Grompe etal. (1993) Nature Genet. 5:111-117].

The nucleotide(s) present at a polymorphic site can be determined by sequencing methods, which may involve initial amplification of the segment to be sequenced. For example, an amplification product can be subjected to automated dideoxy terminator sequencing reactions using a dye-primer cycle sequencing protocol.

Primer extension methods may also be used to determine the nucleotide at a polymorphic site. These methods involve appropriate primers which hybridize just upstream of the polymorphic base of interest in the target nucleic acid. A polymerase is used to specifically extend the 3′ end of the primer with one or more ddNTPs (chain terminators), one of which is complementary to the nucleotide at the polymorphic site. The identity of the incorporated nucleotide is then determined. Identification methods include fluorescence polarization, pyrosequencing and mass spectrometry-based methods.

### EXAMPLE 2

Parametric and Nonparametric Linkage Analysis of Chromosome 10 Microsatellite Markers

Parametric two-point analyses of the genotyping data with respect to the chromosome 10 markers were conducted using affecteds only and two different disease models: an autosomal dominant disease model (disease gene frequency 0.01) and a recessive disease model (disease gene frequency 0.05). Penetrance in affected individuals corresponded to phenocopy rates of 5% for definite AD (n=278), 10% for probable AD (n=645) and 14% for possible AD (n=65). The analyses were performed using the FASTLINK computer program [Cottingham et al. (1993) Am. J. Hum. Genet. 53:252; Schaffer et al. (1994) Hum. Hered 44:225]. The genotyping data were analyzed in terms of the total sample (all families) and for samples stratified by age of onset. Families were considered “late-onset” if all sampled affecteds had onset ages ≧65 years. Families were classifed as APOE ε4/4 positive if at least one affected individual had the ε4/4 genotype, and as APOE ε4/4 negative otherwise. The results of the analyses using the autosomal dominant disease model are provided in TABLE 4, and the results of the analyses for the full dataset using the autosomal recessive disease model are provided in TABLE 5.

TABLE 4
Parametric Analyses - Autosomal Dominant Disease Model

| | | | APOE ε4/4 | APOE ε4/4 |
|---|---|---|---|---|
| Locus Marker | All Families | Late-Onset | Postive | Negative Zₘₐₓ |
| (Location in cM)^{a} | Zₘₐₓ (⊖)^{b} | Zₘₐₓ (⊖) | Zₘₐₓ (P)^{c} | (P) |
| D10S1225 (80.77) | 0.4 (0.32) | 0.9 (0.26) | 0.1 (0.32) | 0.4 (0.30) |
| D10S564 (112.6) | 0 (0.5) | 0 (0.5) | 0 (0.5) | 0 (0.5) |
| D10S583 (115.3) | 3.3 (0.22) | 2.8 (0.21) | 1.2 (0.20) | 2.2 (0.22) |
| D10S1710 (124.3) | 0.7 (0.26) | 0.9 (0.25) | 0.1 (0.28) | 0.7 (0.26) |
| D10S566 (127.1) | 0.8 (0.28) | 0.4 (0.3) | 0.2 (0.32) | 0.7 (0.26) |
| D10S1671 (127.1) | 2.3 (0.22) | 3.4 (0.16) | 0.1 (0.36) | 2.9 (0.18) |
| D10S1741 (128.2) | 0.4 (0.29) | 0.3 (0.32) | 0.3 (0.24) | 0.2 (0.32) |

^{a}Locus marker locations in Kosambi cM according to the Marshfield map.
^{b}Maximum two-point LOD scores (Zₘₐₓ) and the recombination fraction (⊖) used in the calculation.
^{c}Maximum two-point parametric LOD scores (Zₘₐₓ) and the recombination fraction (P) used in the calculation.

TABLE 5
Parametric Analyses - Autosomal Recessive Disease Model

| Locus Marker | All Families | Late-Onset |
|---|---|---|
| (Location in cM) | Zₘₐₓ (P)^{b} | Zₘₐₓ (P) |
| D10S1225 (80.77) | 0.4 (0.36) | 0.9 (0.32) |
| D10S564 (112.6) | 0 (0.50) | 0 (0.50) |
| D10S583 (115.3) | 2.7 (0.28) | 2.4 (0.28) |
| D10S1710 (124.3) | 0.8 (0.32) | 1.1 (0.30) |
| D10S566 (127.1) | 1.1 (0.32) | 0.6 (0.34) |
| D10S1671 (127.1) | 2.9 (0.28) | 3.8 (0.24) |
| D10S1741 (128.2) | 0.4 (0.35) | 0.3 (0.36) |

^{a}Locus marker locations in Kosambi cM according to the Marshfield map.
^{b}Maximum two-point LOD scores (Zₘₐₓ) and the recombination fraction (P) used in the calculation for parametric two-point analyses using an autosomal recessive disease model.

Nonparametric two-point and multipoint analyses of the genotyping data were conducted using the GENEHUNTER-PLUS [Kruglyak et al. (1996) Am. J. Hum. Genet. 58:1347] and ASM (Version 1.0 applying the exponential model) [Kong and Cox (1997) Am. J. Hum. Genet. 61:1179] computer programs. The results of the two-point nonparametric analyses of the full, age-of-onset-stratified, and APOE ε4/4 status-stratified datasets are provided in TABLE 6. The results of multipoint nonparametric analyses of the same datasets are provided in TABLE 7.

TABLE 6
Nonparametric Two-Point Analyses

| Locus Marker | All Families | Late-Onset | APOE ε4/4- |
|---|---|---|---|
| (Location in cM) | Zₗᵣ (p)^{b} | Zₗᵣ(p)^{c} | negative Zₗᵣ (p)^{d} |
| D10S1225 (80.8) | 0.9 (0.2) | 1.6 (0.06) | 0.71 (0.24) |
| D10S564 (112.6) | 0 (1.0) | 0 (1.0) | 0 (1.0) |
| D10S583 (115.3) | 3.0 (0.0015) | 2.8 (0.0025) | 2.5 (0.006) |
| D10S1710 (124.3) | 2.4 (0.008) | 2.8 (0.003) | 2.2 (0.014) |
| D10S566 (127.1) | 1.8 (0.035) | 1.5 (0.06) | 2.1 (0.02) |
| D10S1671 (127.1) | 3.3 (0.0005) | 3.8 | 3.7 (<0.0001) |
| | | (<0.0001) | |
| D10S1741 (128.2) | 1.3 (0.09) | 0.9 (0.2) | 0.8 (0.2) |

^{a}Locus marker locations in Kosambi cM according to the Marshfield map.
^{b}Two-point nonparametric linkage scores (Zₗᵣ = Z scores for the likelihood ratio) and p-values (p).

TABLE 7
Nonparametric Multipoint Analyses

| Locus Marker | All Families | Late-Onset | APOE ε4/4- |
|---|---|---|---|
| (Location in cM) | Zₗᵣ (p)^{b} | Zₗᵣ(p)^{c} | negative Zₗᵣ (p)^{d} |
| D10S1225 (80.8) | 0.9 (0.2) | 1.6 (0.06) | 0.9 (0.2) |
| D10S564 (112.6) | 0.4 (0.4) | 0.5 (0.3) | 0.6 (0.3) |
| D10S583 (115.3) | 1.1 (0.15) | 1.3 (0.1) | 1.3 (0.1) |
| D10S1710 (124.3) | 1.9 (0.029) | 2.1 (0.02) | 2.15 (0.016) |
| D10S566 (127.1) | 1.6 (0.05) | 1.8 (0.03) | 2.0 (0.02) |
| D10S1671 (127.1) | 1.3 (0.01) | 1.6 (0.05) | 1.7 (0.05) |
| D10S1741 (128.2) | 1.2 (0.1) | 1.6 (0.06) | 1.4 (0.08) |

^{a}Locus marker locations in Kosambi cM according to the Marshfield map.
^{b}Maximum multipoint nonparametric linkage scores (Zₗᵣ = Z scores for the likelihood ratio) and p-values (p).

The results of the parametric two-point analyses of seven microsatellite markers on chromosome 10 in 435 AD families using a dominant model revealed significant evidence of linkage of AD to chromosome 10 around marker D10S583 (Zₘₐₓ=3.3) in the full sample and around marker D10S1671 in the late-onset sample (Zₘₐₓ=3.4). The results of the parametric two-point analyses using a recessive model were similar, with a maximium LOD score of 3.8 for marker D10S1671 in the late-onset sample. Although linkage was generally more pronounced in families without the APOE ε4/4 genotype, none of the markers had LOD scores greater than 3 in this stratum.

The results of two-point nonparametric linkage analyses also revealed linkage of AD on chromosome 10q with the highest linkage scores (Zₗᵣ=Z scores for the likelihood ratio) provided by markers D10S583, D10S1710 and D10S1671 (Zₗᵣ scores of 2.8, 2.8 and 3.8, respectively, for the late-onset dataset). Multipoint nonparametric analyses generated maximum Zₗᵣ scores of 1.9 (p=0.029, full sample), 2.1 (p=0.02, late onset) and 2.15 (p=0.016, APOE ε4-negative) at marker D10S1710, which is located between the two markers (i.e., D10S583 and D10S1671) with the greatest linkage signals in the two-point analyses.

### EXAMPLE 3

Association Analyses of a Chromosome 10 Microsatellite Marker

Marker D10S583 was analyzed for association with AD using the Family-Based Association Test computer software program (FBAT) [Rabinowitz and Laird (2000) Hum. Hered. 50:211-223; see also http://www.biostat.harvard.edu/fbat/default.html] to determine if it is within linkage disequilibrium range of an underlying disease gene. The analyses were based on estimated empirical variances (to account for the presence of linkage) [Lake et al. (2000) Am. J. Hum. Genet. 67:1515-1525] as implemented in FBAT (Version 1.0, 1999). Although the multiallelic test on all 11 alleles for marker D10S583 was not significant (p=0.15), the diallelic test revealed significant association of the 211-bp allele with protection against AD (nominal p=0.004, Bonferroni corrected p=0.04). The allele frequency for the 211-bp allele is about 0.096 and the number of informative families contributing to the test statistic was 30.

The results of the linkage and association analyses of markers on human chromosome 10 indicate the presence of a single locus or multiple, e.g., two, loci underlying AD on chromosome 10. The number of AD loci on chromosome 10 can be determined by examination of additional markers distal to marker D10S564, and, in particular, markers in the regions of markers D10S583, D10S1710 and D10S1671. Putative AD gene(s) on chromosome 10 can be identified through further detailed studies of linkage disequilibrium as well as through assessment of candidate genes.

Since modifications will be apparent to those of skill in this art, it is intended that this invention be limited only by the scope of the appended claims.

What is claimed:

1. A method for detecting the presence or absence in a subject of a polymorphism associated with Alzheimer's disease, comprising analyzing chromosome 10 of the subject for a polymorphism associated with Alzheimer's disease.

2. The method of claim 1, wherein the polymorphism is on chromosome 10q.

3. The method of claim 1, wherein the polymorphism is located on chromosome 10q22, 10q23, 10q24, 10q25 or 10q26.

4. The method of claim 1, wherein the polymorphism is located in a region of chromosome 10 about 30 cM around and including D10S583, or about 20 cM around and including D10S583, or about 15 cM around and including D10S583, or about 12 cM around and including D10S583, or about 10 cM around and including D10S583.

5. The method of claim 1, wherein the polymorphism is located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

6. The method of claim 1, wherein the polymorphism is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583, or about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

7. The method of claim 1, wherein the polymorphism is located in one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

8. The method of claim 1, wherein the polymorphism is located in one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

9. The method of claim 1, wherein the polymorphism is an allele of D10S583.

10. A method for detecting the presence or absence in a subject of two or more polymorphisms associated with Alzheimer's disease, comprising analyzing chromosome 10 of the subject for two or more polymorphisms associated with Alzheimer's disease, wherein at least one polymorphism is an allele of D10S583.

11. The method of claim 1, wherein the polymorphism is associated with AD with onset ages of greater than or equal to about 50 years, or greater than or equal to about 60 years, or greater than or equal to about 65 years.

12. The method of claim 1, wherein the polymorphism is associated with an AD DNA segment that has an effect size comparable to or greater than the effect size of APOE.

13. The method of claim 1, wherein the polymorphism is located within an AD DNA segment.

14. The method of claim 1, wherein the association between the polymorphism and Alzheimer's disease is such that it yields a positive result in a family-based test for association.

15. The method of claim 14, wherein the positive result is a P value less than or equal to 0.05.

16. The method of claim 14, wherein the positive result is a P value less than 0.05.

17. A method for indicating a predisposition to or the occurrence of Alzheimer's disease in a subject, comprising:
detecting in nucleic acid obtained from the subject the presence or absence of a polymorphism on chromosome 10 associated with Alzheimer's disease, wherein the presence of the polymorphism is indicative of a predisposition to Alzheimer's disease.

18. A method for confirming a phenotypic diagnosis of Alzheimer's disease in a subject, comprising:
detecting in nucleic acid obtained from the subject the presence or absence of a polymorphism on chromosome 10 associated with Alzheimer's disease, wherein the presence of the polymorphism confirms a phenotypic diagnosis of Alzheimer's disease.

19. The method of claim 17, wherein the polymorphism is located on chromosome 10q.

20. The method of claim 17, wherein the polymorphism is located on chromosome 10q22, 10q23, 10q24, 10q25 or 10q26.

21. The method of claim 17, wherein the polymorphism is located on chromosome 10q22, 10q23 or 10q24.

22. The method of claim 17, wherein the polymorphism is located in a region of chromosome 10 selected from the group consisting of:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

23. The method of claim 17, wherein the polymorphism is located in a region of chromosome 10 selected from the group consisting of:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

24. The method of claim 17, wherein the polymorphism is located about 30 cM around and including D10S583, or about 20 cM around and including D10S583, or about 15 cM around and including D10S583, or about 12 cM around and including D10S583, or about 10 cM around and including D10S583.

25. The method of claim 17, wherein the polymorphism is located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

26. The method of claim 17, wherein the polymorphism is located about 30 Mb around and including D10S583, or about 28 Mb around and including D10S583, or about 20 Mb around and including D10S583, or about 15 Mb around and including D10S583, or about 10 Mb around and including D10S583.

27. The method of claim 17, wherein the polymorphism is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

28. A method for predicting a response of a subject to a drug used to treat Alzheimer's disease, comprising:
detecting the presence or absence of at least one polymorphism on chromosome 10 associated with Alzheimer's disease, wherein the presence of the polymorphism is indicative of an increased or decreased likelihood that the drug treatment for Alzheimer's disease will be effective.

29. The method of claim 28, wherein the polymorphism is located on chromosome 10q.

30. The method of claim 28, wherein the polymorphism is located on chromosome 10q22, 10q23, 10q24, 10q25 or 10q26.

31. The method of claim 28, wherein the polymorphism is located on chromosome 10q22, 10q23 or 10q24.

32. The method of claim 1, wherein the polymorphism is located on chromosome 10q22, 10q23 or 10q24.

33. The method of claim 28, wherein the polymorphism is located about 30 cM around and including D10S583, or about 20 cM around and including D10S583, or about 15 cM around and including D10S583, or about 12 cM around and including D10S583, or about 10 cM around and including D10S583.

34. The method of claim 28, wherein the polymorphism is located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

35. The method of claim 1, wherein the polymorphism is located about 30 Mb around and including D10S583, or about 28 Mb around and including D10S583, or about 20 Mb around and including D10S583, or about 15 Mb around and including D10S583, or about 10 Mb around and including D10S583.

36. The method of claim 1, wherein the polymorphism is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

37. The method of claim 28, wherein the polymorphism is located in a region of chromosome 10 selected from the group consisting of:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

38. The method of claim 28, wherein the polymorphism is located in a region of chromosome 10 selected from the group consisting of:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

39. A method of treating a subject manifesting an Alzheimer's disease phenotype, comprising:
detecting in nucleic acid obtained from the subject the presence of a polymorphism on chromosome 10 associated with Alzheimer's disease, wherein the presence of the polymorphism is indicative of the occurrence of Alzheimer's disease in a subject; and
selecting and administering a treatment that is effective for treatment of Alzheimer's disease.

40. The method of claim 39, wherein the polymorphism is located on chromosome 10q.

41. The method of claim 39, wherein the polymorphism is located on chromosome 10q22, 10q23, 10q24, 10q25 or 10q26.

42. The method of claim 39, wherein the polymorphism is located on chromosome 10q22, 10q23 or 10q24.

43. The method of claim 39, wherein the polymorphism is located about 30 cM around and including D10S583, or about 20 cM around and including D10S583, or about 15 cM around and including D10S583, or about 12 cM around and including D10S583, or about 10 cM around and including D10S583.

44. The method of claim 39, wherein the polymorphism is located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

45. The method of claim 39, wherein the polymorphism is located about 30 Mb around and including D10S583, or about 28 Mb around and including D10S583, or about 20 Mb around and including D10S583, or about 15 Mb around and including D10S583, or about 10 Mb around and including D10S583.

46. The method of claim 39, wherein the polymorphism is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

47. The method of claim 39, wherein the polymorphism is located in a region of chromosome 10 selected from the group consisting of:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

48. The method of claim 39, wherein the polymorphism is located in a region of chromosome 10 selected from the group consisting of:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

49. A method for identifying a gene as a candidate Alzheimer's disease gene, comprising selecting a gene on chromosome 10q that is or encodes a product that has one or more properties which relate to one or more phenomena in neurodegenerative disease and thereby identifying a candidate Alzheimer's disease gene.

50. The method of claim 49, wherein the gene is on chromosome 10q22, 10q23, 10q24, 10q25 or 10q26.

51. The method of claim 49, wherein the gene is on chromosome 10q22, 10q23 or 10q24.

52. The method of claim 49, wherein the gene on chromosome 10q is located about 30 cM around and including D10S583, or about 20 cM around and including D10S583, or about 15 cM around and including D10S583, or about 12 cM around and including D10S583, or about 10 cM around and including D10S583.

53. The method of claim 49, wherein the gene on chromosome 10 is located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

54. The method of claim 49, wherein the gene on chromosome 10 is located about 30 Mb around and including D10S583, or about 28 Mb around and including D10S583, or about 20 Mb around and including D10S583, or about 15 Mb around and including D10S583, or about 10 Mb around and including D10S583.

55. The method of claim 49, wherein the gene on chromsome 10 is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

56. The method of claim 49, wherein the gene on chromosome 10 is located in a region of chromosome 10 selected from the group consisting of:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

57. The method of claim 49, wherein the gene on chromosome 10 is located in a region of chromosome 10 selected from the group consisting of:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

58. The method of claim 49, wherein the one or more phenomena in neurodegenerative disease is selected from the group consisting of senile plaques and components thereof, neuritic plaques, and components thereof, neurofibrillary tangles, tau protein, abnormally phosphorylated tau protein, amyloid precursor protein (APP), processing of APP, Aβ42 protein, α-, β- and γ-secretases, presenilin proteins, amyloid deposition, Lewy bodies, prions, apoptosis, caspases, inflammation, excitotoxicity, excitotoxins, excessive nitric oxide production, oxidative stress, proteases, protease inhibitors, neurotrophic factors, cytokines, calcium-dependent processes, signal transduction, altered ionic homeostasis, altered calcium homeostasis, synaptic molecules, adhesion molecules, molecules involved in membrane turnover, cholesterol and lipid metabolism and transport, cytoskeletal molecules, neuronal proteins, brain proteins, and cell necrosis.

59. A method for identifying a polymorphism associated with Alzheimer's disease, comprising:
analyzing a polymorphism on chromosome 10q for association with Alzheimer's disease.

60. A method for identifying a polymorphism associated with Alzheimer's disease, comprising analyzing a polymorphism on chromosome 10 for association with Alzheimer's disease, wherein the polymorphism is located in the region of 10q22, 10q23, 10q24, 10q25 or 10q26.

61. A method for identifying a polymorphism associated with Alzheimer's disease, comprising analyzing a polymorphism on chromosome 10 for association with Alzheimer's disease, wherein the polymorphism is located in the region of 10q22, 10q23 or 10q24.

62. A method for identifying a polymorphism associated with Alzheimer's disease comprising, analyzing a polymorphism on chromosome 10 for association with Alzheimer's disease, wherein the polymorphism is located is located about 30 cM around and including D10S583, or about 20 cM around and including D10S583, or about 15 cM around and including D10S583, or about 12 cM around and including D10S583, or about 10 cM around and including D10S583.

63. The method of claim 59, wherein the polymorphism is located about 5 cM around and including D10S583, or about 4 cM around and including D10S583, or about 3 cM around and including D10S583, or about 2 cM around D10S583, or about 1 cM around and including D10S583, or about 0.5 cM around and including D1S583, or about 0.25 cM around and including D10S583, or about 0.1 cM around and including D10S583.

64. The method of claim 59, wherein the polymorphism is located about 30 Mb around and including D10S583, or about 28 Mb around and including D10S583, or about 20 Mb around and including D10S583, or about 15 Mb around and including D10S583, or about 10 Mb around and including D10S583.

65. The method of claim 59, wherein the polymorphism is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

66. The method of claim 59, wherein the polymorphism is located in a region of chromosome 10 selected from the group consisting of:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

67. The method of claim 59, wherein the polymorphism is located in a region of chromosome 10 selected from the group consisting of:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

68. The method of claim 59, wherein the association between the polymorphism and Alzheimer's disease yields a positive result in a family-based test for association.

69. The method of claim 68 wherein the positive result is a P value less than or equal to 0.05.

70. The method of claim 68, wherein the positive result is a P value less than 0.05.

71. A combination, comprising:
two or more oligonucleotides that hybridize to, adjacent to, or to DNA flanking a DNA segment on chromosome 10; wherein
the DNA segment comprises a polymorphism that is associated with AD,
at least two of the oligonucleotides hybridize to, adjacent to, or to DNA flanking different DNA segments.

72. The combination of claim 71, wherein the DNA segment is on chromosome 10q.

73. The combination of claim 71, wherein the DNA segment is on chromosome 10q22, 10q23, 10q24, 10q25 or 10q26.

74. The combination of claim 71, wherein the DNA segment is on chromosome 10q22, 10q23 or 10q24.

75. The combination of claim 71, wherein the DNA segment is located in a region of chromosome 10 about 30 cM around and including D10S583, or about 20 cM around and including D10S583, or about 15 cM around and including D10S583, or about 12 cM around and including D10S583, or about 10 cM around and including D10S583.

76. The combination of claim 71, wherein the DNA segment is located in a region of chromosome 10 about 5 cM around and including D10S583, or about 4cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

77. The combination of claim 71, wherein the DNA segment is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

78. The combination of claim 71, wherein the DNA segment is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

79. The combination of claim 71, wherein the DNA segment is located in one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

80. The combination of claim 71, wherein the DNA segment is located in one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

81. The combination of claim 71, wherein the polymorphism is D10S583.

82. A kit, comprising:
two or more oligonucleotides that hybridize to, adjacent to, or to DNA flanking a DNA segment on chromosome 10; and
instructions describing procedures for using the oligonucleotide(s) in the detection of one or more polymorphisms on chromosome 10 associated with AD, wherein
the DNA segment comprises a polymorphic site at which a polymorphism that is associated with AD occurs,
at least two of the oligonucleotides hybridize to, adjacent to, or to DNA flanking different DNA segments.

83. A kit, comprising:
two or more oligonucleotides that hybridize to, adjacent to, or to DNA flanking a DNA segment on chromosome 10; and
instructions describing procedures for using the oligonucleotide(s) in methods of detecting the presence or absence of one or more polymorphisms on chormosome 10 associated with AD, wherein
the DNA segment comprises a polymorphic site at which a polymorphism that is associated with AD occurs,
at least two of the oligonucleotides hybridize to, adjacent to, or to DNA flanking different DNA segments, and
the procedures provide results on which to base a determination of a predisposition to or occurrence of AD in a subject.

84. A kit, comprising:
two or more oligonucleotides that hybridize to, adjacent to, or to DNA flanking a DNA segment on chromosome 10; and
instructions describing procedures for using the oligonucleotide(s) in methods of detecting the presence or absence of one or more polymorphisms on chormosome 10 associated with AD, wherein
the DNA segment comprises a polymorphic site at which a polymorphism that is associated with AD occurs,
at least two of the oligonucleotides hybridize to, adjacent to, or to DNA flanking different DNA segments, and the procedures provide results on which to base a prediction of a subject's response to a treatment for AD.

85. A kit, comprising:
two or more oligonucleotides that hybridize to, adjacent to, or to DNA flanking a DNA segment on chromosome 10; and
instructions describing procedures for using the oligonucleotide(s) in methods of detecting the presence or absence of one or more polymorphisms on chromosome 10 associated with AD,
the DNA segment comprises a polymorphic site at which a polymorphism that is associated with AD occurs,
at least two of the oligonucleotides hybridize to, adjacent to, or to DNA flanking different DNA segments, and
the procedures provide results on which to base a treatment of AD in a subject.

86. A kit, comprising:
one or more oligonucleotides that hybridize to, adjacent to, or to DNA flanking a DNA segment on chromosome 10; and
one or more control samples, wherein
the DNA segment comprises a polymorphic site at which a polymorphism that is associated with AD occurs, and
the one or more control samples comprise the DNA segment in which the polymorphic site does not contain a polymorphism associated with AD and/or the DNA segment in which the polymorphic site does contain a polymorphism associated with AD.

87. The kit of claim 82, wherein the polymorphism or polymorphisms are located on chromosome 10q.

88. The kit of claim 82, wherein the polymorphism or polymorphisms are located on chromosome 10q22, 10q23, 10q24, 10q25 and/or 10q26.

89. The kit of claim 82, wherein the polymorphism or polymorphisms are located on chromosome 10q22, 10q23 and/or 10q24.

90. The kit of claim 82, wherein the polymorphism or polymorphisms are located in a region of chromosome 10 about 30 cM around and including D10S583, or about 20 cM around and including D10S583, or about 15 cM around and including D10S583, or about 12 cM around and including D10S583, or about 10 cM around and including D10S583.

91. The kit of claim 82, wherein the polymorphism or polymorphisms are located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

92. The kit of claim 82, wherein the polymorphism or polymorphisms are located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

93. The kit of claim 82, wherein the polymorphism or polymorphisms are located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

94. The kit of claim 82, wherein the polymorphism or polymorphisms are located one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

95. The kit of claim 82, wherein the polymorphism or polymorphisms are located one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

96. The kit of claim 82, wherein the instructions describe procedures for using the oligonucleotide(s) in methods of detecting the presence or absence of two or more polymorphisms associated with AD.

97. The method of claim 1, wherein the polymorphism is associated with an AD gene that accounts for greater than 1% of the attributtable risk of AD.

98. The method of claim 1, wherein the polymorphism is associated with an AD gene that accounts for greater than 2% of the attributtable risk of AD.

99. The method of claim 1, wherein the polymorphism is associated with an AD gene that accounts for greater than 5% of the attributtable risk of AD.

100. The method of claim 1, wherein the polymorphism is associated with an AD gene that accounts for greater than 10% of the attributtable risk of AD.

101. The method of claim 1, wherein the polymorphism is associated with an AD gene that accounts for greater than 25% of the attributtable risk of AD.

102. A combination comprising:
two or more genetic markers on chromosome 10 that are associated with Alzheimer's disease either individually or as a combination.

103. The combination of claim 102, wherein the genetic markers are located on chromosome 10q.

104. The combination of claim 102, wherein each of the genetic markers is located on chromosome 10q22, 10q23, 10q24, 10q25 or 10q26.

105. The combination of claim 102, wherein each of the genetic markers is located on chromosome 10q22, 10q23 or 10q24.

106. The combination of claim 102, wherein each of the genetic markers is located in a region of chromosome 10 about 30 cM around and including D10S583, or about 20 cM around and including D10S583, or about 15 cM around and including D10S583, or about 12 cM around and including D10S583, or about 10 cM around and including D10S583.

107. The combination of claim 102, wherein each of the genetic markers is located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

108. The combination of claim 102, wherein each of the genetic markers is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

109. The combination of claim 102, wherein each of the genetic markers is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

110. The combination of claim 102, wherein each of the genetic markers is located one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

111. The combination of claim 102, wherein each of the genetic markers is located one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

112. The method of claim 1, wherein the polymorphism is located on chromosome 10q23, 10q24 or 10q25.

113. The method of claim 49, wherein the gene is located on chromosome 10q23, 10q24 or 10q25.

114. The method of claim 1, wherein the polymorphism is located within the PLAU gene.

115. The method of claim 1, wherein the polymorphism is located within the TLL2 gene.

116. The method of claim 1, wherein the polymorphism is located within the PSAP gene.

117. The method of claim 1, wherein the polymorphism is located within the PSD gene.

118. The method of claim 7, wherein the polymorphism is located within the KIAA0904 gene.

119. The method of claim 1, wherein the polymorphism is located within the NFKB2 gene.

120. The method of claim 1, wherein the polymorphism is located within the PPP3CB gene.

121. The method of claim 1, wherein the polymorphism is located within the CH25H gene.

122. The method of claim 1, wherein the polymorphism is located within the FERIL3 gene.

123. The method of claim 17, wherein the polymorphism is located within the PLAU gene.

124. A method for detecting the presence or absence in a subject of polymorphisms associated with Alzheimer's disease, comprising analyzing chromosome 10 of the subject for two or more polymorphisms associated with Alzheimer's disease, wherein at least two of the polymorphisms are associated with different AD DNA segments.

125. The method of claim 124, wherein the two or more polymorphisms are located on chromosome 10q.

126. The method of claim 124, wherein each of the two or more polymorphisms is located on chromosome 10q22, 10q23, 10q24, 10q25 or 10q26.

127. The method of claim 124, wherein each of the two or more polymorphisms is located on chromosome 10q22, 10q23 or 10q24.

128. The method of claim 124, wherein each of the two or more polymorphisms is located on chromosome 10q23, 10q24 or 10q25.

129. The method of claim 124, wherein each of the two or more polymorphisms is located in a region of chromosome 10 about 30 cM around and including D10S583, or about 20 cM around and including D10S583, or about 15 cM around and including D10S583, or about 12 cM around and including D10S583, or about 10 cM around and including D10S583.

130. The method of claim 124, wherein each of the two or more polymorphisms is located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

131. The method of claim 124, wherein each of the two or more polymorphisms is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

132. The method of claim 124, wherein each of the two or more polymorphisms is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

133. The method of claim 124, wherein each of the two or more polymorphisms is located one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

134. The method of claim 124, wherein each of the two or more polymorphisms is located one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

135. A method of determining the level of risk for Alzheimer's disease in a subject, comprising:
analyzing chromosome 10 of the subject for the presence or absence of one or more polymorphisms associated with Alzheimer's disease, wherein the one or more polymorphisms are indicative of an increased or decreased risk for Alzheimer's disease.

136. The method of claim 135, wherein the association between the polymorphism and Alzheimer's disease yields a positive result in a family-based test for association.

137. The method of claim 136, wherein the positive result is a P value less than or equal to 0.05.

138. The method of claim 136, wherein the positive result is a P value less than 0.05.

139. The method of claim 135, wherein the association between the polymorphism and Alzheimer's disease yields a result in a family-based test for association that is indicative of linkage disequilibrium between the polymorphism and an allele associated with Alzheimer's disease.

140. The method of claim 135, wherein the polymorphism is associated with unaffected members of a family having members affected with Alzheimer's disease and is indicative of a decreased risk for Alzheimer's disease.

141. The method of claim 140, wherein the association between the polymorphism and unaffected members of a family having members affected with Alzheimer's disease yields a positive result in a family-based test for association.

142. The method of claim 141, wherein the positive result is a P value less than or equal to 0.05.

143. The method of claim 141, wherein the positive result is a P value less than 0.05.

144. The method of claim 135, wherein the polymorphism is under-represented in cases of a case-control study.

145. The method of claim 135, wherein the polymorphism is an allele of D10S583 and the presence of the allele of D10S583 is indicative of a decreased risk for AD.

146. The method of claim 145, wherein the allele of D10S583 is about 210 bp.

147. The method of claim 145, wherein the allele of D10S583 is 209 bp.

148. The method of claim 145, wherein the allele of D10S583 is 211 bp.

149. The method of claim 135, wherein the polymorphism is associated with affected members of a family having members affected with Alzheimer's disease and is indicative of an increased risk for Alzheimer's disease.

150. The method of claim 149, wherein the association between the polymorphism and affected members of a family having members affected with Alzheimer's disease yields a positive result in a family-based test for association.

151. The method of claim 150, wherein the positive result is a P value less than or equal to 0.05.

152. The method of claim 150, wherein the positive result is a P value less than 0.05.

153. The method of claim 135, wherein the polymorphism is over-represented in cases of a case-control study.

154. The method of claim 135, wherein the one or more polymorphisms are located on chromosome 10q.

155. The method of claim 135, wherein each of the one or more polymorphisms is located on chromosome 10q22, 10q23, 10q24, 10q25 or 10q26.

156. The method of claim 135, wherein each of the one or more polymorphisms is located on chromosome 10q22, 10q23 or 10q24.

157. The method of claim 135, wherein each of the one or more polymorphisms is located on chromosome 10q23, 10q24 or 10q25.

158. The method of claim 135, wherein each of the one or more polymorphisms is located in a region of chromosome 10 about 30 cM around and including D10S583, or about 20 cM around and including D10S583, or about 15 cM around and including D10S583, or about 12 cM around and including D10S583, or about 10 cM around and including D10S583.

159. The method of claim 135, wherein each of the one or more polymorphisms are located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

160. The method of claim 135, wherein each of the one or more polymorphisms is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

161. The method of claim 135, wherein each of the one or more polymorphisms is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

162. The method of claim 135, wherein each of the one or more polymorphisms is located in one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

163. The method of claim 135, wherein each of the one or more polymorphisms is located one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

164. A method for detecting the presence or absence in a subject of a combination of polymorphisms associated with Alzheimer's disease, comprising analyzing chromosome 10 of the subject for a combination of polymorphisms associated with Alzheimer's disease.

165. The method of claim 164, wherein the polymorphisms are located on chromosome 10q.

166. The method of claim 164, wherein each of the polymorphisms is located on chromosome 10q22, 10q23, 10q24, 10q25 or 10q26.

167. The method of claim 164, wherein each of the polymorphisms is located on chromosome 10q22, 10q23 or 10q24.

168. The method of claim 164, wherein each of the polymorphisms is located on chromosome 10q23, 10q24 or 10q25.

169. The method of claim 164, wherein each of the polymorphisms is located about 30 cM around and including D10S583, or about 20 cM around and including D10S583, or about 15 cM around and including D10S583, or about 12 cM around and including D10S583, or about 10 cM around and including D10S583.

170. The method of claim 164, wherein each of the polymorphisms is located about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

171. The method of claim 164, wherein each of the polymorphisms is located about 30 Mb around and including D10S583, or about 28 Mb around and including D10S583, or about 20 Mb around and including D10S583, or about 15 Mb around and including D10S583, or about 10 Mb around and including D10S583.

172. The method of claim 164, wherein each of the polymorphisms is located about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

173. The method of claim 164, wherein each of the polymorphisms is located in one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about-45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

174. The method of claim 164, wherein each of the polymorphisms is located in one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

175. The method of claim 1, wherein the polymorphism is associated with AD with onset ages of greater than or equal to about 65 years.

176. The method of claim 10, wherein the two or more polymorphisms are located on chromosome 10q.

177. The method of claim 10, wherein each of the two or more polymorphisms is located on chromosome 10q22, 10q23 or 10q24.

178. The method of claim 10, wherein each of the two or more polymorphisms is located on chromosome 10q23, 10q24 or 10q25.

179. The method of claim 10, wherein each of the two or more polymorphisms is located in a region of chromosome 10 about 30 cM around and including D10S583, or about 20 cM around and including D10S583, or about 15 cM around and including D10S583, or about 12 cM around and including D10S583, or about 10 cM around and including D10S583.

180. The method of claim 10, wherein each of the two or more polymorphisms is located in a region of chromosome 10 about 5 cM around and including D10S583, or about 4 cM around and including D10S583, or about 3 cM around and including D10S583, or about 2 cM around D10S583, or about 1 cM around and including D10S583, or about 0.5 cM around and including D10S583, or about 0.25 cM around and including D10S583, or about 0.1 cM around and including D10S583.

181. The method of claim 10, wherein each of the two or more polymorphisms is located in a region of chromosome 10 about 30 Mb around and including D10S583, or about 28 Mb around and including D10S583, or about 20 Mb around and including D10S583, or about 15 Mb around and including D10S583, or about 10 Mb around and including D10S583.

182. The method of claim 10, wherein each of the two or more polymorphisms is located in a region of chromosome 10 about 5 Mb around and including D10S583, or about 2.5 Mb around and including D10S583, or about 1 Mb around and including D10S583 or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

183. The method of claim 10, wherein each of the two or more polymorphisms is located one or more of the following regions of chromosome 10:
(a) the region extending about 50 cM, or about 45 cM, or about 33 cM, or about 30 cM, or about 25 cM, or about 21 cM, or about 15 cM, or about 10 cM, or about 5 cM, or about 4 cM, or about 3 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 60 cM, or about 55 cM, or about 50 cM, or about 45 cM, or about 40 cM, or about 35 cM, or about 30 cM, or about 25 cM, or about 20 cM, or about 15 cM, or about 13 cM, or about 12 cM, or about 9 cM, or about 5 cM, or about 2.5 cM, or about 1 cM, or about 0.5 cM or about 0.1 cM distal (telomeric) from and including marker D10S583,
(c) the region extending from and including marker D10S583 to the terminus of the q arm of chromosome 10,
(d) the region extending from and including marker D10S583 to the centromere of chromosome 10,
(e) the region extending about 62 Mb, about 55 Mb, or about 50 Mb, or about 32 Mb, or about 28 Mb, or about 25 Mb, or about 20 Mb, or about 15 Mb, or about 10 Mb, or about 5 Mb, or about 2 Mb, or about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583,
(f) the region extending about 45 Mb, or about 40 Mb, or about 35 Mb, or about 30 Mb, or about 25 Mb, or about 20 Mb, or about 16 Mb, or about 14 Mb, or about 13 Mb, or about 8 Mb, or about 5 Mb, or about 4 Mb, or about 3 Mb, or about 2 Mb, or about 1 Mb, or about 0.5 Mb, or about 0.1 Mb, or about 0.05 Mb, or about 0.01 Mb, or about 1 kb distal from and including marker D10S583,
(g) the region between D10S564 and D10S583, inclusive,
(h) the region between D10S583 and D10S1710, inclusive,
(i) the region between D10S583 and D10S566, inclusive,
(j) the region between D10S583 and D10S1671, inclusive and
(k) the region between D10S583 and D10S1741, inclusive.

184. The method of claim 10, wherein each of the two or more polymorphisms is located one or more of the following regions of chromosome 10:
(a) the region extending about 5 cM, or about 4 cM, or about 2.7 cM, or about 2.5 cM, or about 2 cM, or about 1.5 cM, or about 1 cM, or about 0.5 cM, or about 0.1 cM proximal (centromeric) from and including marker D10S583,
(b) the region extending about 5 cM, or about 2.5 cM, or about 1 cM, or about 0.5 cM or about 0.1 cM distal (telomeric) from and including marker D10S583,
(c) the region extending about 10 Mb, or about 5 Mb, or about 2 Mb, or about 1 Mb, or about 500 kb, or about 300 kb, or about 200 kb, or about 100 kb, or about 80 kb, or about 50 kb, or about 10 kb, or about 5 kb, or about 1 kb proximal from and including marker D10S583; and
(d) the region extending about 10 Mb, or about 8 Mb, or about 5 Mb, or about 4 Mb, or about 3 Mb, or about 2 Mb, or about 1 Mb, or about 500 kb, or about 300 kb, or about 200 kb, or about 100 kb, or about 50 kb, or about 10 kb, or about 5 kb, or about 1 kb distal from and including marker D10S583.

185. The method of claim 10, wherein each of the two or more polymorphisms is located on chromosome 10q22, 10q23, 10q24, 10q25 or 10q26.

186. The method of claim 17, wherein the polymorphism is located on chromosome 10q23, 10q24 or 10q25.

187. The method of claim 18, wherein the polymorphism is located on chromosome 10q23, 10q24 or 10q25.

188. The method of claim 28, wherein the polymorphism is located on chromosome 10q23, 10q24 or 10q25.

189. The method of claim 39, wherein the polymorphism is located on chromosome 10q23, 10q24 or 10q25.

190. The method of claim 60, wherein the polymorphism is located on chromosome 10q23, 10q24 or 10q25.

191. The method of claim 61, wherein the polymorphism is located on chromosome 10q23, 10q24 or 10q25.

192. The method of claim 17, wherein the polymorphism is located within the TLL2 gene.

193. The method of claim 17, wherein the polymorphism is located within the PSAP gene.

194. The method of claim 17, wherein the polymorphism is located within the PSD gene.

195. The method of claim 17, wherein the polymorphism is located within the KIAA0904 gene.

196. The method of claim 17, wherein the polymorphism is located within the NFKB2 gene.

197. The method of claim 17, wherein the polymorphism is located within the PPP3CB gene.

198. The method of claim 17, wherein the polymorphism is located within the CH25H gene.

199. The method of claim 17, wherein the polymorphism is located within the FERIL3 gene.

200. The method of claim 18, wherein the polymorphism is located within the PLAU gene.

201. The method of claim 18, wherein the polymorphism is located within the TLL2 gene.

202. The method of claim 18, wherein the polymorphism is located within the PSAP gene.

203. The method of claim 18, wherein the polymorphism is located within the PSD gene.

204. The method of claim 18, wherein the polymorphism is located within the KIAA0904 gene.

205. The method of claim 18, wherein the polymorphism is located within the NFKB2 gene.

206. The method of claim 18, wherein the polymorphism is located within the PPP3CB gene.

207. The method of claim 18, wherein the polymorphism is located within the CH25H gene.

208. The method of claim 18, wherein the polymorphism is located within the FERIL3 gene.

209. The method of claim 28, wherein the polymorphism is located within the PLAU gene.

210. The method of claim 28, wherein the polymorphism is located within the TLL2 gene.

211. The method of claim 28, wherein the polymorphism is located within the PSAP gene.

212. The method of claim 28, wherein the polymorphism is located within the PSD gene.

213. The method of claim 28, wherein the polymorphism is located within the KIAA0904 gene.

214. The method of claim 28, wherein the polymorphism is located within the NFKB2 gene.

215. The method of claim 28, wherein the polymorphism is located within the PPP3CB gene.

216. The method of claim 28, wherein the polymorphism is located within the CH25H gene.

217. The method of claim 28, wherein the polymorphism is located within the FERIL3 gene.

218. The method of claim 39, wherein the polymorphism is located within the PLAU gene.

219. The method of claim 39, wherein the polymorphism is located within the TLL2 gene.

220. The method of claim 39, wherein the polymorphism is located within the PSAP gene.

221. The method of claim 39, wherein the polymorphism is located within the PSD gene.

222. The method of claim 39, wherein the polymorphism is located within the KIAA0904 gene.

223. The method of claim 39, wherein the polymorphism is located within the NFKB2 gene.

224. The method of claim 39, wherein the polymorphism is located within the PPP3CB gene.

225. The method of claim 39, wherein the polymorphism is located within the CH25H gene.

226. The method of claim 39, wherein the polymorphism is located within the FERIL3 gene.

227. The method of claim 60, wherein the polymorphism is located within the PLAU gene.

228. The method of claim 60, wherein the polymorphism is located within the TLL2 gene.

229. The method of claim 60, wherein the polymorphism is located within the PSAP gene.

230. The method of claim 60, wherein the polymorphism is located within the PSD gene.

231. The method of claim 60, wherein the polymorphism is located within the KIAA0904 gene.

232. The method of claim 60, wherein the polymorphism is located within the NFKB2 gene.

233. The method of claim 60, wherein the polymorphism is located within the PPP3CB gene.

234. The method of claim 60, wherein the polymorphism is located within the CH25H gene.

235. The method of claim 60, wherein the polymorphism is located within the FERIL3 gene.

236. The method of claim 61, wherein the polymorphism is located within the PLAU gene.

237. The method of claim 61, wherein the polymorphism is located within the TLL2 gene.

238. The method of claim 61, wherein the polymorphism is located within the PSAP gene.

239. The method of claim 61, wherein the polymorphism is located within the PSD gene.

240. The method of claim 61, wherein the polymorphism is located within the KIAA0904 gene.

241. The method of claim 61, wherein the polymorphism is located within the NFKB2 gene.

242. The method of claim 61, wherein the polymorphism is located within the PPP3CB gene.

243. The method of claim 61, wherein the polymorphism is located within the CH25H gene.

244. The method of claim 61, wherein the polymorphism is located within the FERIL3 gene.

245. The method of any of claims 1, 17, 18, 28, 39, 59, or 135, wherein the polymorphism is located in a region of chromosome 10 about 1 Mb around and including D10S583, or about 500 kb around and including D10S583, or about 200 kb around and including D10S583, or about 100 kb around and including D10S583, or about 50 kb around and including D10S583, or about 40 kb around and including D10S583, or about 20 kb around and including D10S583, or about 10 kb around and including D10S583, or about 5 kb around and including D10S583, or about 1 kb around and including D10S583.

246. The method of any of claims 1, 17, 18, 28, 39, 59, or 135, wherein the polymorphism is located in a region of chromosome 10 about 0.3 Mb around and including D10S583, or about 0.2 Mb around and including D10S583, or about 0.2 Mb around and including D10S583.

247. The method of any of claims 1, 17, 18, 28, 39, 59, or 135, wherein the polymorphism is located in the region of chromosome 10 extending about 1 Mb, or about 0.5 Mb, or about 0.3 Mb, or about 0.2 Mb, or about 0.1 Mb, or about 0.05 Mb, or about 0.01 Mb, or about 1 kb proximal from and including marker D10S583.

248. The method of any of claims 1, 17, 18, 28, 39, 59, or 135, wherein the polymorphism is located in the region of chromosome 10 extending about 1 Mb, or about 0.5 Mb, or about 0.3 Mb, or about 0.2 Mb, or about 0.1 Mb, or about 0.05 Mb, or about 0.01 Mb, or about 1 kb distal from and including marker D10S583.

249. The method of any of claims 1, 17, 18, 28, 39, 59, or 135, wherein the polymorphism is located in a region of chromosome 10 about 1 cM around and including D10S583, or about 0.5 cM around and including D10S583, or about 0.25 cM around and including D10S583, or about 0.1 cM around and including D10S583.

250. The method of any of claims 1, 17, 18, 28, 39, 59, or 135, wherein the polymorphism is located in the region of chromosome 10 extending about 1 cM, or about 0.5 cM, or about 0.1 cM proximal (centromeric) from and including marker D10S583.

251. The method of any of claims 1, 17, 18, 28, 39, 59, or 135, wherein the polymorphism is located in the region of chromosome 10 extending about 1 cM, or about 0.5 cM, or about 0.1 cM distal (telomeric) from and including marker D10S583.
